SEQUENCE LISTING (131 sequences)

SEQ ID NO: 1 - DNA, 300 bp, Methylobacterium sp.
  source 1..300: mol_type = genomic DNA
acggtcaccccacggactgggcgagtacctcaccggtgttctatcataacgccgagttagttttcgaccgtcccttatgcgatgtaccaccggtgtcggcagccgatttcgtcccaccgggagctggcgttccggttcagaccaccatcatcggtcacgatgtctggattggacacggggccttcatctcccccggcgtgactataggaaacggcgcgatcgtcggggcccaggcggtcgtcacaagagatgtcccaccctatgcggtagttgctggcgtccccgcgaccgtacgacgat

SEQ ID NO: 2 - DNA, 300 bp, Methylobacterium sp.
  source 1..300: mol_type = genomic DNA
ccaataaaagcgttggccgcctgggcaacccgatccgagcctaagactcaaagcgcaagcgaacacttggtagagacagcccgccgactacggcgttccagcactctccggctttgatcggataggcattggtcaaggtgccggtggtgatgacctcgcccgccgcaagcggcgaattactcggatcagcggccagcacctcgaccaagtgtcggagcgcgaccaaagggccacgttcgaggacgtttgaggcgcgaccagtctcgatagtctcatcgtcgcggcgaagctgcacctcga

SEQ ID NO: 3 - DNA, 300 bp, Methylobacterium sp.
  source 1..300: mol_type = genomic DNA
cgatggcaccgacctgccatgcctctgccgtccgcgccagaatggtaaagaggacgaagggggtaaggatcgtcgctgcagtgttgagcagcgaccagagaagggggccgaacatcggcatcaaacctcgattgccactcggacgcgaagcgcgtcttgaaggagggatggaagcgaaacggccgcagagtaaccgccgacgaaagattgcacccctcatcgagcaggatcggaggtgaaggcaagcgtgggttattggtaagtgcaaaaaatataatggtagcgtcagatctagcgttc

SEQ ID NO: 4 - DNA, 23 bp, synthetic construct
  source 1..23: mol_type = other DNA
cctcaccggtgttctatcataac

SEQ ID NO: 5 - DNA, 20 bp, synthetic construct
  source 1..20: mol_type = other DNA
ccgatgatggtggtctgaac

SEQ ID NO: 6 - DNA, 21 bp, synthetic construct
  source 1..21: mol_type = other DNA
cgtcccttatgcgatgtacca

SEQ ID NO: 7 - DNA, 22 bp, synthetic construct
  source 1..22: mol_type = other DNA
gatccgagcctaagactcaaag

SEQ ID NO: 8 - DNA, 21 bp, synthetic construct
  source 1..21: mol_type = other DNA
gaccaatgcctatccgatcaa

SEQ ID NO: 9 - DNA, 20 bp, synthetic construct
  source 1..20: mol_type = other DNA
aacacttggtagagacagcc

SEQ ID NO: 10 - DNA, 21 bp, synthetic construct
  source 1..21: mol_type = other DNA
aaggagggatggaagcgaaac

SEQ ID NO: 11 - DNA, 19 bp, synthetic construct
  source 1..19: mol_type = other DNA
ataacccacgcttgccttc

SEQ ID NO: 12 - DNA, 21 bp, synthetic construct
  source 1..21: mol_type = other DNA
cgcagagtaaccgccgacgaa

SEQ ID NO: 13 - DNA, 300 bp, Methylobacterium sp.
  source 1..300: mol_type = genomic DNA
gcaaaacgacctaatagttctacagcggcatgcgccaagtcagcgcggtgaacagtatacctgggagcaacttgtcctccgaaacccacataaaacaaattactcctggcagtgcccagtccatcaaaatcgaatacaatatttctcgaggaggcatctgtaatagcctgccaaagcaacaaagctatggcgccgttatgactttcattgcttctggtagacataaaataatatgccgatttgtgatcccaaatgtagaatattgccgcatcaattgcgccaagtttatttcggatcgat

SEQ ID NO: 14 - DNA, 300 bp, Methylobacterium sp.
  source 1..300: mol_type = genomic DNA
ggcgccaacggtatgatcgcatgattttcctgcggcatagcttgcgggaatggcgtatttggcgctctcctcaggaatttctaagggcatacgcaggaactctacagcacttttactggtattttgtagtgacagcggaggaggctggtgctcaaggtaatcgtgatgaagtgatccgggccattcggggcgcgtttctagtctttccaatccgcgccctgtaccacgtattacgccggaccggtctgcgccgcgccgccctcttgaccgccctaaatgtctaagagcgtctaacaaagc

SEQ ID NO: 15 - DNA, 300 bp, Methylobacterium sp.
  source 1..300: mol_type = genomic DNA
gacgatatcgctcatcttcactgcattgaagctggtgccgtactgcatagggatgaaaaagtgatgcggatagacggctgacgggaaagcgcctggtcgatcgaagactttgctgacgaggttgtggtagccccggatataggcatcgaaggccgggacgttgatcccatcctttgccttatcttgactggcgtcgtcgcgtgccgtcagaacgggcacgtcgcaggtcatcgaggccagcaccttgcggaacacctgcgttccgccgttgggattatcgacggcgaacgcggtggccgc

SEQ ID NO: 16 - DNA, 21 bp, synthetic construct
  source 1..21: mol_type = other DNA
gtcctccgaaacccacataaa

SEQ ID NO: 17 - DNA, 23 bp, synthetic construct
  source 1..23: mol_type = other DNA
ctaccagaagcaatgaaagtcat

SEQ ID NO: 18 - DNA, 22 bp, synthetic construct
  source 1..22: mol_type = other DNA
tctgtaatagcctgccaaagca

SEQ ID NO: 19 - DNA, 19 bp, synthetic construct
  source 1..19: mol_type = other DNA
ggctggtgctcaaggtaat

SEQ ID NO: 20 - DNA, 20 bp, synthetic construct
  source 1..20: mol_type = other DNA
acatttagggcggtcaagag

SEQ ID NO: 21 - DNA, 19 bp, synthetic construct
  source 1..19: mol_type = other DNA
atgaagtgatccgggccat

SEQ ID NO: 22 - DNA, 21 bp, synthetic construct
  source 1..21: mol_type = other DNA
ccgtactgcatagggatgaaa

SEQ ID NO: 23 - DNA, 21 bp, synthetic construct
  source 1..21: mol_type = other DNA
taaggcaaaggatgggatcaa

SEQ ID NO: 24 - DNA, 21 bp, synthetic construct
  source 1..21: mol_type = other DNA
ttgctgacgaggttgtggtag

SEQ ID NO: 25 - DNA, 300 bp, Methylobacterium sp.
  source 1..300: mol_type = genomic DNA
caactatgtagacccgacggtgcgatttcacttcgcaaagccgcagggcagcacccttgcgctcaatgttgacgccagcgtgatctatactattaccgtcacgcacacgcagggcggcgtacagattcatcgcgagagtaagaaccaccatcagaccatcacgcgcagcgacctgagcaagcagttcggcgttggtgtggccgaccagctgacgcgcgatcaggtcatgaaggtgatcgagtcggcatttcgcgacgctacccgctaagatcggcgcccacgaaacgctacgagactagg

SEQ ID NO: 26 - DNA, 300 bp, Methylobacterium sp.
  source 1..300: mol_type = genomic DNA
agccggcatcttgttcaaggcgctcacctcgacgccgacgctgtaggcgacttgagagggcgtctcatatgaacgaagcatcttcgcgtagagaaccttcttgttctcctgcgtgatgttcgctttgcagacgttgactgccgccatgaacgccgaagccttgcgcgcttcatcgtaatcgcctgcgaaggcgggtagtgaaaagcttagtgcaatggcaaacacagccgccgaacgtcgcatggtatccgtccccgattgacggcagtgccgccatatctcggctttagcagagctgat

SEQ ID NO: 27 - DNA, 300 bp, Methylobacterium sp.
  source 1..300: mol_type = genomic DNA
aacctgcgccggccgaggtttcgcgagccgtcgccacgggcaacgcctcgcccgcgatgtgcaaaaaagtccccggcacttcgcgccgtcgtccgatccacgaccgcgaatttctcaacgagtacaaggtgcttatgggagatccgagcgtccgtcccggagcccgagaccgcgcggcccgagtaataggcgaaaaagactcctactcctcgggcttctcgggccccctcagcaacatctacgcttgccgcccatcaccctggcgggagatcagcgacgagacacaggcccacttcgccc

SEQ ID NO: 28 - DNA, 22 bp, synthetic construct
  source 1..22: mol_type = other DNA
ttgacgccagcgtgatctatac

SEQ ID NO: 29 - DNA, 22 bp, synthetic construct
  source 1..22: mol_type = other DNA
gtgatggtctgatggtggttct

SEQ ID NO: 30 - DNA, 19 bp, synthetic construct
  source 1..19: mol_type = other DNA
tattaccgtcacgcacacg

SEQ ID NO: 31 - DNA, 24 bp, synthetic construct
  source 1..24: mol_type = other DNA
cttcgcgtagagaaccttcttgtt

SEQ ID NO: 32 - DNA, 22 bp, synthetic construct
  source 1..22: mol_type = other DNA
cttcgcaggcgattacgatgaa

SEQ ID NO: 33 - DNA, 20 bp, synthetic construct
  source 1..20: mol_type = other DNA
cgtgatgttcgctttgcaga

SEQ ID NO: 34 - DNA, 23 bp, synthetic construct
  source 1..23: mol_type = other DNA
ccgcgaatttctcaacgagtaca

SEQ ID NO: 35 - DNA, 21 bp, synthetic construct
  source 1..21: mol_type = other DNA
gcccgaggagtaggagtcttt

SEQ ID NO: 36 - DNA, 20 bp, synthetic construct
  source 1..20: mol_type = other DNA
aggtgcttatgggagatccg

SEQ ID NO: 37 - DNA, 300 bp, Methylobacterium sp.
  source 1..300: mol_type = genomic DNA
agtcattgatcaagcaacccctattgagttggatatcgaaggatcaaggtcgcgtcaatagatgcatctatcaggccaaatgtcgcttttcaagaatggctctttcgaagctatctttataatcgctcgccattctctcattaccaaaatcgaccttaactagctcgacattgatgcgagcagctccggcaaacgaggagagattgaccttaaaggaattgaacgcctcaagcaattcagacacattaccaggagtgctatagcaacaaccagacccatatcggtcaataacctctttta

SEQ ID NO: 38 - DNA, 300 bp, Methylobacterium sp.
  source 1..300: mol_type = genomic DNA
cgcaaaacgatttatcactgccatcttgttgtttgataacccttttttaccagacgttatgctgggcgagaaagaggactagcagatcggagcggtatcgcgatttttcggtagttcgcgcctacaacaggataagatccgatagtgaagcaacatggctgttttttgatttgtaagtcagcaacttaagcagccagcctatctgccgtcgcagacgcttgaggcatcgggcagcatcttagaaaaggtggcagtaattgccacagcggaacgtagcggcacggataagcacgcagggtc

SEQ ID NO: 39 - DNA, 300 bp, Methylobacterium sp.
  source 1..300: mol_type = genomic DNA
cccatctggacccaatatccccttcatcgacaattcccgagtaagtgtgggttcgaggatttcgcgaaacagccttgttcgttcctccggccttaaaattggcgtgccgtcgggagatcgataggcatcccttacctgcctttcgaccgccggcacacgcgcgccggtcgtcgtgttcacggccacggaatggacgaaggtgcgccgctcatttcgctcgtttgccgtctccaccatccaggaggccagcaggacggtttcgtctcgaccgccggtcacacacaccgcaagggactcagg

SEQ ID NO: 40 - DNA, 22 bp, synthetic construct
  source 1..22: mol_type = other DNA
tcgctcgccattctctcattac

SEQ ID NO: 41 - DNA, 22 bp, synthetic construct
  source 1..22: mol_type = other DNA
aggtcaatctctcctcgtttgc

SEQ ID NO: 42 - DNA, 18 bp, synthetic construct
  source 1..18: mol_type = other DNA
tcgacattgatgcgagca

SEQ ID NO: 43 - DNA, 22 bp, synthetic construct
  source 1..22: mol_type = other DNA
ttcgcgcctacaacaggataag

SEQ ID NO: 44 - DNA, 23 bp, synthetic construct
  source 1..23: mol_type = other DNA
cagataggctggctgcttaagtt

SEQ ID NO: 45 - DNA, 18 bp, synthetic construct
  source 1..18: mol_type = other DNA
tccgatagtgaagcaaca

SEQ ID NO: 46 - DNA, 24 bp, synthetic construct
  source 1..24: mol_type = other DNA
gagtaagtgtgggttcgaggattt

SEQ ID NO: 47 - DNA, 23 bp, synthetic construct
  source 1..23: mol_type = other DNA
aggtaagggatgcctatcgatct

SEQ ID NO: 48 - DNA, 18 bp, synthetic construct
  source 1..18: mol_type = other DNA
cggaggaacgaacaaggc

SEQ ID NO: 49 - DNA, 300 bp, Methylobacterium sp.
  source 1..300: mol_type = genomic DNA
acctgctaaaatcacgtcctctcagattgaaaaatcattgaagaaacgtgtcgaacgattgccggggattatgacgttagatcaattgaaaaatacaagctttgaaattgagttacagccaaaagatgccccggatccggacccatcagacttcggtggctagttcgagccaaactcgaacgtcgccatggcgcgcaagtcgcaataccatttcacagcgcagcggttatttcgttgtacactgtagcaatgcgtcggcttgcgcgcttccgctggcgatcaaaggtccgccgatttacg

SEQ ID NO: 50 - DNA, 300 bp, Methylobacterium sp.
  source 1..300: mol_type = genomic DNA
tcccgaacatacaatggaggaagcgtgtggtaggccaatttgtaacgaaatatggcatcggtcacggctctctcaataaattcgatctcaagtcttctgaacgagcatgcctcatccttatcctgagcgaacgcctgccagtttgcagtcattccaacatacatagccaaaaaggcgaggtagaccttcatacgggcacctcaatcgtccccattcgttcaagctccttcaagataacagccgcaccacattgctgagatcgaagattcggatcaaatattccatcaaatttatactttc

SEQ ID NO: 51 - DNA, 300 bp, Methylobacterium sp.
  source 1..300: mol_type = genomic DNA
gcatcctttgcgctcgcaggcctaaggtcaagcccggttacttcgtttggtagaacgaggtagacgatgcctagtcttaaggtggcccatgttaaccaacagggccagaacatgattatagttccgttagatgccaacttcggttacaaaaccgatggtgagcagtccgacatcatgttcgaaatacaggacgcggcgcggtccgccggtcttgcgggtgccgtagtagcgttctggcagtcaggtggacaaacccgtttccggggcccggctccgtggcacccattccttcgcagcctc

SEQ ID NO: 52 - DNA, 22 bp, synthetic construct
  source 1..22: mol_type = other DNA
gcgcaagtcgcaataccatttc

SEQ ID NO: 53 - DNA, 21 bp, synthetic construct
  source 1..21: mol_type = other DNA
cgtaaatcggcggacctttga

SEQ ID NO: 54 - DNA, 19 bp, synthetic construct
  source 1..19: mol_type = other DNA
cgcagcggttatttcgttg

SEQ ID NO: 55 - DNA, 22 bp, synthetic construct
  source 1..22: mol_type = other DNA
acgagcatgcctcatccttatc

SEQ ID NO: 56 - DNA, 21 bp, synthetic construct
  source 1..21: mol_type = other DNA
cgattgaggtgcccgtatgaa

SEQ ID NO: 57 - DNA, 20 bp, synthetic construct
  source 1..20: mol_type = other DNA
tgccagtttgcagtcattcc

SEQ ID NO: 58 - DNA, 23 bp, synthetic construct
  source 1..23: mol_type = other DNA
cccggttacttcgtttggtagaa

SEQ ID NO: 59 - DNA, 24 bp, synthetic construct
  source 1..24: mol_type = other DNA
cgaagttggcatctaacggaacta

SEQ ID NO: 60 - DNA, 20 bp, synthetic construct
  source 1..20: mol_type = other DNA
tggcccatgttaaccaacag

SEQ ID NO: 61 - DNA, 300 bp, Methylobacterium sp.
  source 1..300: mol_type = genomic DNA
gcccttctgtcaggcgatattgtataatggcgttgccccaatagaagcagccattcgtgcgagggcagcagcgacgctaggtcgaaagagcatcctaatctcgatcaagatgcgactgagatttctgatgaaaatatctagacacaagcaaagctggtgaaattacaacgatcatggcgacaattgcggccaattcggccggaacttgaaggaacataaaaatgaatattacaaatataccgcaaagcatgtagagttgctacaccaagggtcgggacgtccaaaaaaactcactgagga

SEQ ID NO: 62 - DNA, 300 bp, Methylobacterium sp.
  source 1..300: mol_type = genomic DNA
ggaacataaaaatgaatattacaaatataccgcaaagcatgtagagttgctacaccaagggtcgggacgtccaaaaaaactcactgaggaagtcgactggaagcacgaggcgccccccccaggagcggggcgaccggcaagggggcccgcaattgtcgccatgatcgaccagcttaggtaggatcctctttcgacctaacgaatggctgcttctattggggcaacgccattatacaatatcgcctgaccatctggaacgcggcccggtccaccggcaggttggcgacgacagcgtcggag

SEQ ID NO: 63 - DNA, 300 bp, Methylobacterium sp.
  source 1..300: mol_type = genomic DNA
cggcgtcgaccagccgggcgaactgcttgggcatgctctcccgcgacgccggccacagccgcgtccccgtccctccgcacaggatcatcgggtggatttgaaaggcaaaacgggacatcaggataggccgctcaggcgttggcgctgaggcgcttgatgtcggcgtcgaccatctcggtgatcagcgcctcgaggctggtctcggcctcccagccgaaggtcgccttggccttggcggggttgcccagcagcacctcgacctctgccggccggaacagcgccgggtcgacgatcaggtgg

SEQ ID NO: 64 - DNA, 300 bp, Methylobacterium sp.
  source 1..300: mol_type = genomic DNA
ctggacatgcgcccaccccggccaagtccgaccgcaccggcaaccgctcctgtagtcgtcgtcatcgttctcacccctgaggcggagaccgtccgctaacggggtgtctcaagcaaccgtggggcggaggaacacgcacgtagtcgcgtttcaaggttcgcacgaacgcctcggccatgccgttgctctgcgggctctccagcggcgtcgtttttggcaccaaaccaaggtcgcgggcgaagcggcgcgtgtcgcggggactgtcaggaatttcgtgtgggggcggccatagtggatccg

SEQ ID NO: 65 - DNA, 300 bp, Methylobacterium sp.
  source 1..300: mol_type = genomic DNA
ggaaatcggcttcaagtacgacgtcacgccggccatgcaggtcacgggtgcactgttcaatctcgagcgcgacaaccagccgttcccctcgaacgtggagtccggcctcgtccttggcgcaggtcagacacgcacccagggcgcggaaatcggcctggccggctatctaaccgattggtggcaggtctttggcggctacgcttataccgaggcacgcgtactctcgccactggaagacgatggagacgtgatcgcagcaggtaatctcgtcggcaacgttccgctaaatactttcagtct

SEQ ID NO: 66 - DNA, 300 bp, Methylobacterium sp.
  source 1..300: mol_type = genomic DNA
cggcctggccggctatctaaccgattggtggcaggtctttggcggctacgcttataccgaggcacgcgtactctcgccactggaagacgatggagacgtgatcgcagcaggtaatctcgtcggcaacgttccgctaaatactttcagtctgttcaacaagttcgatatcaacgagaatttctccgttgctctgggctattactatcaggatgccagctttgcctcctcagacaatgcagtgcgtttgccaagttattcgcggttcgatggcgggttgttctatcgattcgacgagttgac

SEQ ID NO: 67 - DNA, 300 bp, Methylobacterium sp.
  source 1..300: mol_type = genomic DNA
acgttccgctaaatactttcagtctgttcaacaagttcgatatcaacgagaatttctccgttgctctgggctattactatcaggatgccagctttgcctcctcagacaatgcagtgcgtttgccaagttattcgcggttcgatggcgggttgttctatcgattcgacgagttgacacgcgttcagcttagcgtcgagaacattttcgacaggcgttacatcatcaactccaacaacaacaacaacctcacgcctggcgcgccgagaacagtccgcgtgcaattgatcgctcggttctaaa

SEQ ID NO: 68 - DNA, 300 bp, Methylobacterium sp.
  source 1..300: mol_type = genomic DNA
agcccacaagcctgatgcacttaactacatcctctaatgtcgcgccaatttgcttggcggcaggggatgttgtatcgtcataggcttgtctaaccggaacttgtttgccaatctctttggcgatcgcaaccgccatctcgtgttcgtcaaccatgtgcgcgttcctctaattgcactcatggtgccacgtgcacctccgatcgtctcgtgtctagaatgaaggtgggaacaaccttacacaggctttcgcgacgcgcgaatttctggtttctccgcctcggatgtgggtttgagcgcttc

SEQ ID NO: 69 - DNA, 300 bp, Methylobacterium sp.
  source 1..300: mol_type = genomic DNA
cttttcatttgtcatgatctcgaccaaggtattcacggcaagctcggtctgttgcttagcaagtgcctgaacttcgcgaacgatcggctctcgacccttcgggttcgagacctgtcccttttgaaaaccacgtgccctacacttttcgggatcaaggtgcgggttggctttggtcaaaattctctggcgtcccattacacgccctccgcatcatcgttcccgcgaacgatctgacccccgacttccgcgaggaagcgtgtggcgtgatcctcgaagcggaatgccacctcgaactgttcc

SEQ ID NO: 70 - DNA, 300 bp, Methylobacterium sp.
  source 1..300: mol_type = genomic DNA
cagcagcaagcagatcgttgaaaaccgcttgaaccgcatcttgatcgggaccggaaccaatcaggtcatctaggtaaaccgagacgtaaactcgtttgcgctcggcatctttcagaacgtccgtgatgccagaccgcattagtaccatcgtcgccaaggcgggcgactgaacgaagccgatcggcagagagtaacggggaccgcccctaatcgggttgcgaacgcaagaccacttagcaaaggttcgagcacggccgaacttcgcatggtggagagccgcggcaacacggttccgtgata

SEQ ID NO: 71 - DNA, 300 bp, Methylobacterium sp.
  source 1..300: mol_type = genomic DNA
tagacattccaacaaaccggcaagaggctcgtcctcactcgaggatttgttgggacttgcatgatgtcgaagcggagccgttatgacctgggtgcgatcatgcgccgagcatgggagatggctcgggaggcggcattcgcggttggcgagcgggcacggactcaccttgctgccgcgatgcgcagcgcgtgggccgaagccaagttggcactcgcgcccacgaagacggagcaggatcgtctctctccgagcgacatgatcggacatgaggacgcctaccaaggccgggttctaaaatat

SEQ ID NO: 72 - DNA, 300 bp, Methylobacterium sp.
  source 1..300: mol_type = genomic DNA
aagatggatacgacaagcgcgattacattatttgcgaaatagatggacaaataaaagacaaaggactgatgtatttccttaaatctggacaagttgacctctttcacatagaagtcaccactccctttgggacaatttggtgtcacgaaaacatagaggccgaacttcttagctgaattatcgcgctccgggttcttatgcggctgagtgaagcgcgggacagcttgcgagcagggccgccaatggcagccgggatgacacaatgctcggtctcccgacgcttcttcaatcgggagcgct

SEQ ID NO: 73 - DNA, 299 bp, Methylobacterium sp.
  source 1..299: mol_type = genomic DNA
agctgaattatcgcgctccgggttcttatgcggctgagtgaagcgcgggacagcttgcgagcagggccgccaatggcagccgggatgacacaatgctcggtctcccgacgcttcttcaatcgggagcgcttcgcagcccggggcggcgcgctcatgcgtcacgacctgggccctgcgcaccttcgcggccccgccgtcccggcagatccctgatgccccaagtgggcggccactccatcaaagaaccccggcctgtggcagatctcgtaggcataccgaggttccgcagtgcccccacc

SEQ ID NO: 74 - DNA, 300 bp, Methylobacterium sp.
  source 1..300: mol_type = genomic DNA
accgaaggcgtccccggacacgaaggcctgaaacaccatatctgtggcgatcaggccgacgtggtcgcggacttcaactggcagagaatgccaggccgcttcgatttcagatgatactggtacggacataggagcggcttagctttctcagtgcaaatgtgattgattccggctcaaaaatgatcttgatcggacgagacgttttcaatccatgtcgtgttgccatcgccgatcggtgcgtcaagagacagatggcgccgaccgtagatacgcgttcgggttgcccgcaccgcttctcca

SEQ ID NO: 75 - DNA, 300 bp, Methylobacterium sp.
  source 1..300: mol_type = genomic DNA
ggaggtgtgatctgatgatgtgctggatgaaattggcggtcgagcacttgttcagcttggccagctcgacgagatcggcgtgatgctcggcgtcgatcaggatgttcagcgagaccggacgtacgcaggacttggtattagcgccgttgcgcatcagcttgcagccttgctctgcttctcagcgtgccgcgtcaggatgaccctgatgtagctgttgaggttgatgccgtaatagcctgcggactctgtgagatcccggcgaagatcgtcggcgagggtcaggcggatggtgctggtcgg

SEQ ID NO: 76 - DNA, 300 bp, Methylobacterium sp.
  source 1..300: mol_type = genomic DNA
aagtaaccgctcaacatgatcttcagcatgttgtccaacagcaggagaatacatgtaattcaccatgaccggcaagctgcgactggccattgcttccaccgcttgaatgtagcgatcgaatttcgcaaaatcagggtggaatgaaaatatcgaaccaaactgcgagccttgaatccgttctgcaaaattatcgaaaaattttcttggccgactgccgttcgaaaacattcttacgtttacatgcggcccgcctgaaacaagacagtctaccagctctgggaaatgggggtgaagggtcgg

SEQ ID NO: 77 - protein, 279 aa, synthetic construct
  VARIANT 1..63: note = X can be any amino acid residue or can be absent.
  VARIANT 67: note = X can be any amino acid residue or can be absent.
  VARIANT 70..77: note = X can be any amino acid residue or can be absent.
  VARIANT 79..80: note = X can be any amino acid residue or can be absent.
  VARIANT 105..135: note = X can be any amino acid residue or can be absent.
  VARIANT 141..151: note = X can be any amino acid residue or can be absent.
  VARIANT 153: note = X can be any amino acid residue or can be absent.
  VARIANT 155: note = X can be any amino acid residue or can be absent.
  VARIANT 158..159: note = X can be any amino acid residue or can be absent.
  VARIANT 163: note = X can be any amino acid residue or can be absent.
  VARIANT 166: note = X can be any amino acid residue or can be absent.
  VARIANT 174: note = X can be any amino acid residue or can be absent.
  VARIANT 179: note = X can be any amino acid residue or can be absent.
  VARIANT 189: note = X can be any amino acid residue or can be absent.
  VARIANT 195..216: note = X can be any amino acid residue or can be absent.
  VARIANT 222..230: note = 222..230
  VARIANT 233: note = X can be any amino acid residue or can be absent.
  VARIANT 236..240: note = X can be any amino acid residue or can be absent.
  VARIANT 242..279: note = X can be any amino acid residue or can be absent.
XXXXXXXXXXXXXXXXXXXXXXXXXXXXXXXXXXXXXXXXXXXXXXXXXXXXXXXXXXXXXXXMPTXLPXXXXXXXXRXXPVRRLSWPDTARFLILVARVRLLDXXXXXXXXXXXXXXXXXXXXXXXXXXXXXXXLRLHAXXXXXXXXXXXVXRXGSXXAGDXLLXLMRRWLAXHEAIXALLPGVPEPXHVAQVXXXXXXXXXXXXXXXXXXXXXXRAILQXXXXXXXXXVPXSRXXXXXPXXXXXXXXXXXXXXXXXXXXXXXXXXXXXXXXXXXXXX

SEQ ID NO: 78 - protein, 154 aa, synthetic construct
  VARIANT 1..7: note = X can be any amino acid residue or can be absent.
  VARIANT 9..10: note = X can be any amino acid residue or can be absent.
  VARIANT 19: note = X can be any amino acid residue or can be absent.
  VARIANT 52: note = X can be any amino acid residue or can be absent.
  VARIANT 71: note = X can be any amino acid residue or can be absent.
  VARIANT 74: note = X can be any amino acid residue or can be absent.
  VARIANT 94: note = X can be any amino acid residue or can be absent.
  VARIANT 96..154: note = X can be any amino acid residue or can be absent.
XXXXXXXMXXPLRRTVQVXEDGRMNLPADMRRVLGLTGAGRVILTQDEDGIXITTAEQALKRVRSLAAPFXRGXGSVVDEFIAERRADAAREDXEXXXXXXXXXXXXXXXXXXXXXXXXXXXXXXXXXXXXXXXXXXXXXXXXXXXXXXXXXXX

SEQ ID NO: 79 - protein, 103 aa, synthetic construct
  VARIANT 2..29: note = X can be any amino acid residue or can be absent.
  VARIANT 42: note = X can be any amino acid residue or can be absent.
  VARIANT 69..72: note = X can be any amino acid residue or can be absent.
  VARIANT 77..103: note = X can be any amino acid residue or can be absent.
MXXXXXXXXXXXXXXXXXXXXXXXXXXXXPQSYALQILAIAXAMSVLGLGGVWIASRIYDRNTRRLEAXXXXRRGDXXXXXXXXXXXXXXXXXXXXXXXXXXX

SEQ ID NO: 80 - protein, 2503 aa, synthetic construct
  VARIANT 1..7: note = X can be any amino acid residue or can be absent.
  VARIANT 12..27: note = X can be any amino acid residue or can be absent.
  VARIANT 29..33: note = X can be any amino acid residue or can be absent.
  VARIANT 39: note = X can be any amino acid residue or can be absent.
  VARIANT 42: note = X can be any amino acid residue or can be absent.
  VARIANT 46..47: note = X can be any amino acid residue or can be absent.
  VARIANT 50: note = X can be any amino acid residue or can be absent.
  VARIANT 58: note = X can be any amino acid residue or can be absent.
  VARIANT 60: note = X can be any amino acid residue or can be absent.
  VARIANT 62..63: note = X can be any amino acid residue or can be absent.
  VARIANT 65: note = X can be any amino acid residue or can be absent.
  VARIANT 71: note = X can be any amino acid residue or can be absent.
  VARIANT 73: note = X can be any amino acid residue or can be absent.
  VARIANT 75: note = X can be any amino acid residue or can be absent.
  VARIANT 77: note = X can be any amino acid residue or can be absent.
  VARIANT 79: note = X can be any amino acid residue or can be absent.
  VARIANT 81..87: note = X can be any amino acid residue or can be absent.
  VARIANT 89: note = X can be any amino acid residue or can be absent.
  VARIANT 92: note = X can be any amino acid residue or can be absent.
  VARIANT 95..98: note = X can be any amino acid residue or can be absent.
  VARIANT 101: note = X can be any amino acid residue or can be absent.
  VARIANT 108: note = X can be any amino acid residue or can be absent.
  VARIANT 112..116: note = X can be any amino acid residue or can be absent.
  VARIANT 119..120: note = X can be any amino acid residue or can be absent.
  VARIANT 123..126: note = X can be any amino acid residue or can be absent.
  VARIANT 130: note = X can be any amino acid residue or can be absent.
  VARIANT 132..133: note = X can be any amino acid residue or can be absent.
  VARIANT 135..142: note = X can be any amino acid residue or can be absent.
  VARIANT 144: note = X can be any amino acid residue or can be absent.
  VARIANT 148..149: note = X can be any amino acid residue or can be absent.
  VARIANT 162..163: note = X can be any amino acid residue or can be absent.
  VARIANT 182: note = X can be any amino acid residue or can be absent.
  VARIANT 186: note = X can be any amino acid residue or can be absent.
  VARIANT 189: note = X can be any amino acid residue or can be absent.
  VARIANT 192: note = X can be any amino acid residue or can be absent.
  VARIANT 195: note = X can be any amino acid residue or can be absent.
  VARIANT 197: note = X can be any amino acid residue or can be absent.
  VARIANT 200..201: note = X can be any amino acid residue or can be absent.
  VARIANT 205: note = X can be any amino acid residue or can be absent.
  VARIANT 220: note = X can be any amino acid residue or can be absent.
  VARIANT 222..226: note = X can be any amino acid residue or can be absent.
  VARIANT 230..241: note = X can be any amino acid residue or can be absent.
  VARIANT 244..249: note = X can be any amino acid residue or can be absent.
  VARIANT 266: note = X can be any amino acid residue or can be absent.
  VARIANT 268..269: note = X can be any amino acid residue or can be absent.
  VARIANT 276: note = X can be any amino acid residue or can be absent.
  VARIANT 289: note = X can be any amino acid residue or can be absent.
  VARIANT 302..303: note = X can be any amino acid residue or can be absent.
  VARIANT 306: note = X can be any amino acid residue or can be absent.
  VARIANT 312: note = X can be any amino acid residue or can be absent.
  VARIANT 315..321: note = X can be any amino acid residue or can be absent.
  VARIANT 324: note = X can be any amino acid residue or can be absent.
  VARIANT 326: note = X can be any amino acid residue or can be absent.
  VARIANT 336..347: note = X can be any amino acid residue or can be absent.
  VARIANT 358..366: note = X can be any amino acid residue or can be absent.
  VARIANT 370..371: note = X can be any amino acid residue or can be absent.
  VARIANT 373..374: note = X can be any amino acid residue or can be absent.
  VARIANT 376..378: note = X can be any amino acid residue or can be absent.
  VARIANT 381..382: note = X can be any amino acid residue or can be absent.
  VARIANT 385: note = X can be any amino acid residue or can be absent.
  VARIANT 388: note = X can be any amino acid residue or can be absent.
  VARIANT 390: note = X can be any amino acid residue or can be absent.
  VARIANT 394: note = X can be any amino acid residue or can be absent.
  VARIANT 400: note = X can be any amino acid residue or can be absent.
  VARIANT 402..403: note = X can be any amino acid residue or can be absent.
  VARIANT 406: note = X can be any amino acid residue or can be absent.
  VARIANT 410: note = X can be any amino acid residue or can be absent.
  VARIANT 413..420: note = X can be any amino acid residue or can be absent.
  VARIANT 423..430: note = X can be any amino acid residue or can be absent.
  VARIANT 432..434: note = X can be any amino acid residue or can be absent.
  VARIANT 436: note = X can be any amino acid residue or can be absent.
  VARIANT 438..446: note = X can be any amino acid residue or can be absent.
  VARIANT 452: note = X can be any amino acid residue or can be absent.
  VARIANT 455..459: note = X can be any amino acid residue or can be absent.
  VARIANT 462..467: note = X can be any amino acid residue or can be absent.
  VARIANT 469..470: note = X can be any amino acid residue or can be absent.
  VARIANT 474: note = X can be any amino acid residue or can be absent.
  VARIANT 477: note = X can be any amino acid residue or can be absent.
  VARIANT 503: note = X can be any amino acid residue or can be absent.
  VARIANT 508: note = X can be any amino acid residue or can be absent.
  VARIANT 511: note = X can be any amino acid residue or can be absent.
  VARIANT 513: note = X can be any amino acid residue or can be absent.
  VARIANT 516..517: note = X can be any amino acid residue or can be absent.
  VARIANT 521..522: note = X can be any amino acid residue or can be absent.
  VARIANT 525..529: note = X can be any amino acid residue or can be absent.
  VARIANT 531..557: note = X can be any amino acid residue or can be absent.
  VARIANT 559..561: note = X can be any amino acid residue or can be absent.
  VARIANT 563: note = X can be any amino acid residue or can be absent.
  VARIANT 566..583: note = X can be any amino acid residue or can be absent.
  VARIANT 587: note = X can be any amino acid residue or can be absent.
  VARIANT 590: note = X can be any amino acid residue or can be absent.
  VARIANT 598..599: note = X can be any amino acid residue or can be absent.
  VARIANT 612: note = X can be any amino acid residue or can be absent.
  VARIANT 620..621: note = X can be any amino acid residue or can be absent.
  VARIANT 623..627: note = X can be any amino acid residue or can be absent.
  VARIANT 636: note = X can be any amino acid residue or can be absent.
  VARIANT 639: note = X can be any amino acid residue or can be absent.
  VARIANT 650..651: note = X can be any amino acid residue or can be absent.
  VARIANT 656: note = X can be any amino acid residue or can be absent.
  VARIANT 671: note = X can be any amino acid residue or can be absent.
  VARIANT 673: note = X can be any amino acid residue or can be absent.
  VARIANT 677..680: note = X can be any amino acid residue or can be absent.
  VARIANT 683: note = X can be any amino acid residue or can be absent.
  VARIANT 685: note = X can be any amino acid residue or can be absent.
  VARIANT 692: note = X can be any amino acid residue or can be absent.
  VARIANT 710..711: note = X can be any amino acid residue or can be absent.
  VARIANT 730: note = X can be any amino acid residue or can be absent.
  VARIANT 733: note = X can be any amino acid residue or can be absent.
  VARIANT 751: note = X can be any amino acid residue or can be absent.
  VARIANT 757..759: note = X can be any amino acid residue or can be absent.
  VARIANT 761: note = X can be any amino acid residue or can be absent.
  VARIANT 764: note = X can be any amino acid residue or can be absent.
  VARIANT 766: note = X can be any amino acid residue or can be absent.
  VARIANT 770: note = X can be any amino acid residue or can be absent.
  VARIANT 775..776: note = X can be any amino acid residue or can be absent.
  VARIANT 779..783: note = X can be any amino acid residue or can be absent.
  VARIANT 786: note = X can be any amino acid residue or can be absent.
  VARIANT 816..825: note = X can be any amino acid residue or can be absent.
  VARIANT 829: note = X can be any amino acid residue or can be absent.
  VARIANT 838..839: note = X can be any amino acid residue or can be absent.
  VARIANT 857..858: note = X can be any amino acid residue or can be absent.
  VARIANT 863..864: note = X can be any amino acid residue or can be absent.
  VARIANT 869..870: note = X can be any amino acid residue or can be absent.
  VARIANT 877: note = X can be any amino acid residue or can be absent.
  VARIANT 883: note = X can be any amino acid residue or can be absent.
  VARIANT 886: note = X can be any amino acid residue or can be absent.
  VARIANT 893..894: note = X can be any amino acid residue or can be absent.
  VARIANT 896..902: note = X can be any amino acid residue or can be absent.
  VARIANT 905: note = X can be any amino acid residue or can be absent.
  VARIANT 907..908: note = X can be any amino acid residue or can be absent.
  VARIANT 911..913: note = X can be any amino acid residue or can be absent.
  VARIANT 916: note = X can be any amino acid residue or can be absent.
  VARIANT 922: note = X can be any amino acid residue or can be absent.
  VARIANT 926..927: note = X can be any amino acid residue or can be absent.
  VARIANT 929: note = X can be any amino acid residue or can be absent.
  VARIANT 936: note = X can be any amino acid residue or can be absent.
  VARIANT 938: note = X can be any amino acid residue or can be absent.
  VARIANT 944..947: note = X can be any amino acid residue or can be absent.
  VARIANT 949..952: note = X can be any amino acid residue or can be absent.
  VARIANT 954: note = X can be any amino acid residue or can be absent.
  VARIANT 956: note = X can be any amino acid residue or can be absent.
  VARIANT 959..970: note = X can be any amino acid residue or can be absent.
  VARIANT 973..977: note = X can be any amino acid residue or can be absent.
  VARIANT 980: note = X can be any amino acid residue or can be absent.
  VARIANT 982: note = X can be any amino acid residue or can be absent.
  VARIANT 985..989: note = X can be any amino acid residue or can be absent.
  VARIANT 991..993: note = X can be any amino acid residue or can be absent.
  VARIANT 996: note = X can be any amino acid residue or can be absent.
  VARIANT 998: note = X can be any amino acid residue or can be absent.
  VARIANT 1006: note = X can be any amino acid residue or can be absent.
  VARIANT 1008..1009: note = X can be any amino acid residue or can be absent.
  VARIANT 1016: note = X can be any amino acid residue or can be absent.
  VARIANT 1019..1020: note = X can be any amino acid residue or can be absent.
  VARIANT 1023..1024: note = X can be any amino acid residue or can be absent.
  VARIANT 1026: note = X can be any amino acid residue or can be absent.
  VARIANT 1030..1034: note = X can be any amino acid residue or can be absent.
  VARIANT 1038..1039: note = X can be any amino acid residue or can be absent.
  VARIANT 1046..1047: note = X can be any amino acid residue or can be absent.
  VARIANT 1049..1050: note = X can be any amino acid residue or can be absent.
  VARIANT 1053: note = X can be any amino acid residue or can be absent.
  VARIANT 1055..1056: note = X can be any amino acid residue or can be absent.
  VARIANT 1058: note = X can be any amino acid residue or can be absent.
  VARIANT 1060..1061: note = X can be any amino acid residue or can be absent.
  VARIANT 1063..1065: note = X can be any amino acid residue or can be absent.
  VARIANT 1067: note = X can be any amino acid residue or can be absent.
  VARIANT 1069: note = X can be any amino acid residue or can be absent.
  VARIANT 1073: note = X can be any amino acid residue or can be absent.
  VARIANT 1076: note = X can be any amino acid residue or can be absent.
  VARIANT 1079..1080: note = X can be any amino acid residue or can be absent.
  VARIANT 1082..1084: note = X can be any amino acid residue or can be absent.
  VARIANT 1090: note = X can be any amino acid residue or can be absent.
  VARIANT 1093..1095: note = X can be any amino acid residue or can be absent.
  VARIANT 1097..1099: note = X can be any amino acid residue or can be absent.
  VARIANT 1103..1104: note = X can be any amino acid residue or can be absent.
  VARIANT 1107..1109: note = X can be any amino acid residue or can be absent.
  VARIANT 1111..1112: note = X can be any amino acid residue or can be absent.
  VARIANT 1114: note = X can be any amino acid residue or can be absent.
  VARIANT 1116..1118: note = X can be any amino acid residue or can be absent.
  VARIANT 1121..1122: note = X can be any amino acid residue or can be absent.
  VARIANT 1128..1129: note = X can be any amino acid residue or can be absent.
  VARIANT 1132: note = X can be any amino acid residue or can be absent.
  VARIANT 1136: note = X can be any amino acid residue or can be absent.
  VARIANT 1140: note = X can be any amino acid residue or can be absent.
  VARIANT 1143: note = X can be any amino acid residue or can be absent.
  VARIANT 1146..1148: note = X can be any amino acid residue or can be absent.
  VARIANT 1150: note = X can be any amino acid residue or can be absent.
  VARIANT 1156..1157: note = X can be any amino acid residue or can be absent.
  VARIANT 1159: note = X can be any amino acid residue or can be absent.
  VARIANT 1166..1167: note = X can be any amino acid residue or can be absent.
  VARIANT 1169: note = X can be any amino acid residue or can be absent.
  VARIANT 1172..1191: note = X can be any amino acid residue or can be absent.
  VARIANT 1193: note = X can be any amino acid residue or can be absent.
  VARIANT 1195..1197: note = X can be any amino acid residue or can be absent.
  VARIANT 1199: note = X can be any amino acid residue or can be absent.
  VARIANT 1202..1204: note = X can be any amino acid residue or can be absent.
  VARIANT 1206..1209: note = X can be any amino acid residue or can be absent.
  VARIANT 1214..1215: note = X can be any amino acid residue or can be absent.
  VARIANT 1218..1219: note = X can be any amino acid residue or can be absent.
  VARIANT 1221: note = X can be any amino acid residue or can be absent.
  VARIANT 1223: note = X can be any amino acid residue or can be absent.
  VARIANT 1228: note = X can be any amino acid residue or can be absent.
  VARIANT 1231..1232: note = X can be any amino acid residue or can be absent.
  VARIANT 1235..1236: note = X can be any amino acid residue or can be absent.
  VARIANT 1238: note = X can be any amino acid residue or can be absent.
  VARIANT 1241..1243: note = X can be any amino acid residue or can be absent.
  VARIANT 1245: note = X can be any amino acid residue or can be absent.
  VARIANT 1247: note = X can be any amino acid residue or can be absent.
  VARIANT 1250..1251: note = X can be any amino acid residue or can be absent.
  VARIANT 1253..1258: note = X can be any amino acid residue or can be absent.
  VARIANT 1261: note = X can be any amino acid residue or can be absent.
  VARIANT 1263..1265: note = X can be any amino acid residue or can be absent.
  VARIANT 1267..1277: note = X can be any amino acid residue or can be absent.
  VARIANT 1280: note = X can be any amino acid residue or can be absent.
  VARIANT 1283: note = X can be any amino acid residue or can be absent.
  VARIANT 1285..1287: note = X can be any amino acid residue or can be absent.
  VARIANT 1289..1291: note = X can be any amino acid residue or can be absent.
  VARIANT 1293..1294: note = X can be any amino acid residue or can be absent.
  VARIANT 1301: note = X can be any amino acid residue or can be absent.
  VARIANT 1305..1313: note = X can be any amino acid residue or can be absent.
  VARIANT 1319: note = X can be any amino acid residue or can be absent.
  VARIANT 1322..1325: note = X can be any amino acid residue or can be absent.
  VARIANT 1328..1329: note = X can be any amino acid residue or can be absent.
  VARIANT 1331..1452: note = X can be any amino acid residue or can be absent.
  VARIANT 1454: note = X can be any amino acid residue or can be absent.
  VARIANT 1457..1458: note = X can be any amino acid residue or can be absent.
  VARIANT 1464: note = X can be any amino acid residue or can be absent.
  VARIANT 1467..1469: note = X can be any amino acid residue or can be absent.
  VARIANT 1471..1472: note = X can be any amino acid residue or can be absent.
  VARIANT 1476: note = X can be any amino acid residue or can be absent.
  VARIANT 1478: note = X can be any amino acid residue or can be absent.
  VARIANT 1482: note = X can be any amino acid residue or can be absent.
  VARIANT 1485..1486: note = X can be any amino acid residue or can be absent.
  VARIANT 1488: note = X can be any amino acid residue or can be absent.
  VARIANT 1493: note = X can be any amino acid residue or can be absent.
  VARIANT 1497: note = X can be any amino acid residue or can be absent.
  VARIANT 1501: note = X can be any amino acid residue or can be absent.
  VARIANT 1503: note = X can be any amino acid residue or can be absent.
  VARIANT 1505..1506: note = X can be any amino acid residue or can be absent.
  VARIANT 1508..1511: note = X can be any amino acid residue or can be absent.
  VARIANT 1515: note = X can be any amino acid residue or can be absent.
  VARIANT 1524..1526: note = X can be any amino acid residue or can be absent.
  VARIANT 1530..1531: note = X can be any amino acid residue or can be absent.
  VARIANT 1534..1538: note = X can be any amino acid residue or can be absent.
  VARIANT 1541: note = X can be any amino acid residue or can be absent.
  VARIANT 1550: note = X can be any amino acid residue or can be absent.
  VARIANT 1552..1553: note = X can be any amino acid residue or can be absent.
  VARIANT 1560: note = X can be any amino acid residue or can be absent.
  VARIANT 1563..1565: note = X can be any amino acid residue or can be absent.
  VARIANT 1570..1576: note = X can be any amino acid residue or can be absent.
  VARIANT 1583..1589: note = X can be any amino acid residue or can be absent.
  VARIANT 1591: note = X can be any amino acid residue or can be absent.
  VARIANT 1595: note = X can be any amino acid residue or can be absent.
  VARIANT 1604: note = X can be any amino acid residue or can be absent.
  VARIANT 1607..1611: note = X can be any amino acid residue or can be absent.
  VARIANT 1615: note = X can be any amino acid residue or can be absent.
  VARIANT 1625: note = X can be any amino acid residue or can be absent.
  VARIANT 1635..1637: note = X can be any amino acid residue or can be absent.
  VARIANT 1652: note = X can be any amino acid residue or can be absent.
  VARIANT 1654: note = X can be any amino acid residue or can be absent.
  VARIANT 1656: note = X can be any amino acid residue or can be absent.
  VARIANT 1708..1711: note = X can be any amino acid residue or can be absent.
  VARIANT 1714: note = X can be any amino acid residue or can be absent.
  VARIANT 1735: note = X can be any amino acid residue or can be absent.
  VARIANT 1737..1741: note = X can be any amino acid residue or can be absent.
  VARIANT 1757: note = X can be any amino acid residue or can be absent.
  VARIANT 1761: note = X can be any amino acid residue or can be absent.
  VARIANT 1763: note = X can be any amino acid residue or can be absent.
  VARIANT 1775: note = X can be any amino acid residue or can be absent.
  VARIANT 1781..1782: note = X can be any amino acid residue or can be absent.
  VARIANT 1784: note = X can be any amino acid residue or can be absent.
  VARIANT 1792..1793: note = X can be any amino acid residue or can be absent.
  VARIANT 1809: note = X can be any amino acid residue or can be absent.
  VARIANT 1812: note = X can be any amino acid residue or can be absent.
  VARIANT 1815..1823: note = X can be any amino acid residue or can be absent.
  VARIANT 1827: note = X can be any amino acid residue or can be absent.
  VARIANT 1829: note = X can be any amino acid residue or can be absent.
  VARIANT 1851: note = X can be any amino acid residue or can be absent.
  VARIANT 1855..1858: note = X can be any amino acid residue or can be absent.
  VARIANT 1863: note = X can be any amino acid residue or can be absent.
  VARIANT 1867..1869: note = X can be any amino acid residue or can be absent.
  VARIANT 1871: note = X can be any amino acid residue or can be absent.
  VARIANT 1881: note = X can be any amino acid residue or can be absent.
  VARIANT 1896: note = X can be any amino acid residue or can be absent.
  VARIANT 1899..1900: note = X can be any amino acid residue or can be absent.
  VARIANT 1902: note = X can be any amino acid residue or can be absent.
  VARIANT 1906..1909: note = X can be any amino acid residue or can be absent.
  VARIANT 1920..1922: note = X can be any amino acid residue or can be absent.
  VARIANT 1944: note = X can be any amino acid residue or can be absent.
  VARIANT 1952..1953: note = X can be any amino acid residue or can be absent.
  VARIANT 1965: note = X can be any amino acid residue or can be absent.
  VARIANT 1969..1970: note = X can be any amino acid residue or can be absent.
  VARIANT 1973: note = X can be any amino acid residue or can be absent.
  VARIANT 1976..1977: note = X can be any amino acid residue or can be absent.
  VARIANT 1979: note = X can be any amino acid residue or can be absent.
  VARIANT 1992: note = X can be any amino acid residue or can be absent.
  VARIANT 1997..2004: note = X can be any amino acid residue or can be absent.
  VARIANT 2007: note = X can be any amino acid residue or can be absent.
  VARIANT 2012: note = X can be any amino acid residue or can be absent.
  VARIANT 2014..2015: note = X can be any amino acid residue or can be absent.
  VARIANT 2021: note = X can be any amino acid residue or can be absent.
  VARIANT 2049..2051: note = X can be any amino acid residue or can be absent.
  VARIANT 2053: note = X can be any amino acid residue or can be absent.
  VARIANT 2066: note = X can be any amino acid residue or can be absent.
  VARIANT 2076: note = X can be any amino acid residue or can be absent.
  VARIANT 2085: note = X can be any amino acid residue or can be absent.
  VARIANT 2087: note = X can be any amino acid residue or can be absent.
  VARIANT 2092: note = X can be any amino acid residue or can be absent.
  VARIANT 2096..2097: note = X can be any amino acid residue or can be absent.
  VARIANT 2100: note = X can be any amino acid residue or can be absent.
  VARIANT 2109..2165: note = X can be any amino acid residue or can be absent.
  VARIANT 2167..2178: note = X can be any amino acid residue or can be absent.
  VARIANT 2180..2186: note = X can be any amino acid residue or can be absent.
  VARIANT 2188..2191: note = X can be any amino acid residue or can be absent.
  VARIANT 2193..2209: note = X can be any amino acid residue or can be absent.
  VARIANT 2211..2212: note = X can be any amino acid residue or can be absent.
  VARIANT 2217..2218: note = X can be any amino acid residue or can be absent.
  VARIANT 2220..2222: note = X can be any amino acid residue or can be absent.
  VARIANT 2224..2225: note = X can be any amino acid residue or can be absent.
  VARIANT 2230..2231: note = X can be any amino acid residue or can be absent.
  VARIANT 2233..2235: note = X can be any amino acid residue or can be absent.
  VARIANT 2240: note = X can be any amino acid residue or can be absent.
  VARIANT 2242: note = X can be any amino acid residue or can be absent.
  VARIANT 2245..2246: note = X can be any amino acid residue or can be absent.
  VARIANT 2252: note = X can be any amino acid residue or can be absent.
  VARIANT 2256..2257: note = X can be any amino acid residue or can be absent.
  VARIANT 2260: note = X can be any amino acid residue or can be absent.
  VARIANT 2265..2266: note = X can be any amino acid residue or can be absent.
  VARIANT 2270..2271: note = X can be any amino acid residue or can be absent.
  VARIANT 2275..2276: note = X can be any amino acid residue or can be absent.
  VARIANT 2278..2279: note = X can be any amino acid residue or can be absent.
  VARIANT 2283..2286: note = X can be any amino acid residue or can be absent.
  VARIANT 2288: note = X can be any amino acid residue or can be absent.
  VARIANT 2290..2291: note = X can be any amino acid residue or can be absent.
  VARIANT 2298: note = X can be any amino acid residue or can be absent.
  VARIANT 2300..2301: note = X can be any amino acid residue or can be absent.
  VARIANT 2303..2308: note = X can be any amino acid residue or can be absent.
  VARIANT 2313..2322: note = X can be any amino acid residue or can be absent.
  VARIANT 2325..2327: note = X can be any amino acid residue or can be absent.
  VARIANT 2329: note = X can be any amino acid residue or can be absent.
  VARIANT 2332: note = X can be any amino acid residue or can be absent.
  VARIANT 2335: note = X can be any amino acid residue or can be absent.
  VARIANT 2340: note = X can be any amino acid residue or can be absent.
  VARIANT 2342..2353: note = X can be any amino acid residue or can be absent.
  VARIANT 2356: note = X can be any amino acid residue or can be absent.
  VARIANT 2359..2367: note = X can be any amino acid residue or can be absent.
  VARIANT 2370: note = X can be any amino acid residue or can be absent.
  VARIANT 2373..2374: note = X can be any amino acid residue or can be absent.
  VARIANT 2376..2406: note = X can be any amino acid residue or can be absent.
  VARIANT 2414: note = X can be any amino acid residue or can be absent.
  VARIANT 2416: note = X can be any amino acid residue or can be absent.
  VARIANT 2418: note = X can be any amino acid residue or can be absent.
  VARIANT 2423..2503: note = X can be any amino acid residue or can be absent.
XXXXXXXDTLEXXXXXXXXXXXXXXXXRXXXXXLACTVXDHXSIAXXQNXVPIIRDIXLXNXXDXDLADVXLXIXAXPXLXRPLTLXIXRIXAGXXXXIDXPDLRIDXAILXXXXXAGXXESXXXXVTLXLXXSXXXXXXXXEXAREXXDLRLLPPSHWGGXXAAPELLAAFVRPNDPAVDXILRXAAXILXRAXRXTAXXDGYXSGRKARAWEMAEAIXAXXXXXAMAXXXXXXXXXXXXRIXXXXXXYVLPPASFERSGQKVRXPXXIVERRLXTCLDLTLLWAACXEQAGLNPLLVLTXXHAXLGLWLXDEXXXXXXXDDXQXLRKRRDLQEXXXXXXXXXXXXLILIETTILTXXXXXXXXXDPPXXFXXAXXXGAXXIDXDAXAXLEMXLDLRRXRXXGIXPLDXGEXXXXXXXXAPXXXXXXXXLXXXQXLXXXXXXXXXAPPSFXEDXXXXXIDXXXXXXPXXRLEXWKXRLLDLTLRNKLLNFKPGKGSLTLDCXEPGAXEDXLXAGXXFRLXXRPXXXXXDXXXXXXXXXXXXXXXXXXXXXXXXXXXAXXXRXEIXXXXXXXXXXXXXXXXXXELEXRLXDLFRLARXXFEEGGANVLFLAXGFLTWTRXXGXXXXXRAPLLLVPXALXRASVRAGFRLXXHDEEXRLNPTLLEMLRQDFXLXMPDXXXXLPXDXSGIDVEXIWRIVRTHIRDLKGWEVXXEVVLSAFSFTKFLMWKDLXERXDLLKRSPVVRHLLDTPKXAYGDGXXXTXFPXPXRLDXEHPPXXIFXXXXXPLXADSSQLSAILAAASGKDFVLFGPPGTGKSXXXXXXXXXXQTIXNMIAQCLAXXGRTVLFVSQKSAALEVVXXRRRLXXVGLGXXCLEVHAXKAQKTXVIXQLREAWXXRXXXXXXXWDXAXXDLXXXREXLNGVVXSLHXXRXNGLSAHXAXGRVIAXXXXGXXXXLXLXWPXXXXXXXXXXXXSLXXXXXRAXCXELXXXXXLXXXVGXIXDHPLRGIXAXXWSPLWRXEMXXAIXXLXRTLXXXXXSGQXXAEAMGLXXLXXTYXGXXRXLXXLXXXLXRXEARXGLXFLXXGXXXLRQAVXARXXXQXXXARLXXRLXXXYXXPXVXXXDLXXLLAEWXXAKXSNFXLRGXRLXRVXXXLXPFAQGXXPXDIGPDLXXLXEIXXXXXXXXXXXXXXXXXXXXVXEXXXAXLGXXXPXXXXWSDPXXPAXXFXAXMAWAXRLXXVIXXMXPLXXXGXDXVRXXLXXXXXXLDXEXXXLXXXXXXXXXXXGGXLAXAXXXFXXXRXXAVKAIEXLGRXXXXXXXXXLAGRAXPDXXXXPVXXEXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXDXWVXXTLAVAXRWXXXLXXKAQXWXAWQXAAXXAXKAGLXPLVXAIEXGXIXXDXXXXAFEXAYARWWIDXXXTDDXXLRXXXXXFMXQRHEEAIRXFXXADSRLSXLAXXXVRARXXXXXXXIGGGVPXXXXXXXAXAFGXDPEWGTLAXEIXXXXXTKRXRHMPLRQLFXRMPNALTRLXXXTPCLMMSPLSIAQYXPXEXKPFDIVIFDEASQIAPWDAIGAIARGRQVVIVGDPEQLPPTNVGDRGVDEIXXXXDGXDVADQESILDECLAANLPQRXLXXXXXWHYRSRHESLIAFSNXHYYXGXLVTFPSPVTDDXRAVRLXXVXDGLYERGXXRVNRPEARALVAEVVXRLXDPXXXXXXXXXAFAXEXRSLGIVTFNGEQQRLIENLLDXERRXXXXPELEXFFDXXXWXEPVFVKNLEXVQGDERDAILFSVAXGPXXDXTGRXXXXISSLNREGGHXXXRRLNVAITRARRELVVFASMRXDQVDLGRXXARGVRDFKHFLXFAEXXGAXALXXAXAPTGGDIESPFEXAVMAXXXXXXXXALXARGWXIXXQVGVSXFRIDLGIVHPDAPGRYLAGVECDGATYXXXHXAATARDRDRLREXVLTDLGWRIXRVWSTDWWXDXQGALXRLDXXLRXDLDADRAKXXXXXXXXXXXXXXXXXXXXXXXXXXXXXXXXXXXXXXXXXXXXXXXXXXXXXXXXXPXXXXXXXXXXXXPXXXXXXXQXXXXPXXXXXXXXXXXXXXXXXYXXADLSXXGXXXDXXRFHDXXYXXXLAAMXAXVVXXEGPVFXDILXXRLXRAHGXXRITXXLRQXXLXXVDPXXXXTXEXXRIVLWPXGXXPXXXXXXFRPAXXXXXXXXXXRAXXXDXPLXELXGLARXLXXXXXXXXXXXXMAXRLXXXXXXXXXGLXRMXXAXXXXXXXXXXXXXXXXXXXXXXXXXXXXXXXRARFAEAXAXLXARESXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 81 - protein, 409 aa, synthetic construct
  VARIANT 1..84: note = X can be any amino acid residue or can be absent.
  VARIANT 105: note = X can be any amino acid residue or can be absent.
  VARIANT 115: note = X can be any amino acid residue or can be absent.
  VARIANT 117: note = X can be any amino acid residue or can be absent.
  VARIANT 130: note = X can be any amino acid residue or can be absent.
  VARIANT 132: note = X can be any amino acid residue or can be absent.
  VARIANT 143..144: note = X can be any amino acid residue or can be absent.
  VARIANT 153..159: note = X can be any amino acid residue or can be absent.
  VARIANT 173..208: note = X can be any amino acid residue or can be absent.
  VARIANT 220..226: note = X can be any amino acid residue or can be absent.
  VARIANT 238..247: note = X can be any amino acid residue or can be absent.
  VARIANT 260: note = X can be any amino acid residue or can be absent.
  VARIANT 267..269: note = X can be any amino acid residue or can be absent.
  VARIANT 285: note = X can be any amino acid residue or can be absent.
  VARIANT 298: note = X can be any amino acid residue or can be absent.
  VARIANT 300..301: note = X can be any amino acid residue or can be absent.
  VARIANT 303: note = X can be any amino acid residue or can be absent.
  VARIANT 305..306: note = X can be any amino acid residue or can be absent.
  VARIANT 320..322: note = X can be any amino acid residue or can be absent.
  VARIANT 324: note = X can be any amino acid residue or can be absent.
  VARIANT 327..328: note = X can be any amino acid residue or can be absent.
  VARIANT 330: note = X can be any amino acid residue or can be absent.
  VARIANT 333..335: note = X can be any amino acid residue or can be absent.
  VARIANT 349: note = X can be any amino acid residue or can be absent.
  VARIANT 368..382: note = X can be any amino acid residue or can be absent.
  VARIANT 386..409: note = X can be any amino acid residue or can be absent.
XXXXXXXXXXXXXXXXXXXXXXXXXXXXXXXXXXXXXXXXXXXXXXXXXXXXXXXXXXXXXXXXXXXXXXXXXXXXXXXXXXXXMQTILYARVSTADQTIAHQRXQAEAAGFKIXDXVVADEGVSGVSTXLXDRPQGRRLFDXXMLRRGDVLXXXXXXXVVRWVDRLGRNYAXXXXXXXXXXXXXXXXXXXXXXXXXXXXXXXXXXXXDVTETIREFMRXXXXXXXRGVIVRTVINNXXXXXXXXXXMTFDGATTDPMQXAVRDALXXXIGFMAATAQAQAEATXKEAQKAGIEHAKXRXXEXDXXAYRGRKPSYTREQXXXDXVRXXLXQGXXXVSAIAKATGLSRQXTVYRIRDNPAEAEAALARXXXXXXXXXXXXXXXWAAXXXXXXXXXXXXXXXXXXXXXXXX

SEQ ID NO: 82 - protein, 218 aa, synthetic construct
  VARIANT 2..19: note = X can be any amino acid residue or can be absent.
  VARIANT 23: note = X can be any amino acid residue or can be absent.
  VARIANT 25..26: note = X can be any amino acid residue or can be absent.
  VARIANT 47: note = X can be any amino acid residue or can be absent.
  VARIANT 51..54: note = X can be any amino acid residue or can be absent.
  VARIANT 56..60: note = X can be any amino acid residue or can be absent.
  VARIANT 62: note = X can be any amino acid residue or can be absent.
  VARIANT 74..130: note = X can be any amino acid residue or can be absent.
  VARIANT 136..140: note = X can be any amino acid residue or can be absent.
  VARIANT 144..145: note = X can be any amino acid residue or can be absent.
  VARIANT 147..161: note = X can be any amino acid residue or can be absent.
  VARIANT 163: note = X can be any amino acid residue or can be absent.
  VARIANT 166..218: note = X can be any amino acid residue or can be absent.
MXXXXXXXXXXXXXXXXXXYDDXIXXADAAAGEERDAIMRALAEDMXEASXXXXRXXXXXGXFVRAERPADLAXXXXXXXXXXXXXXXXXXXXXXXXXXXXXXXXXXXXXXXXXXXXXXXXXXXXXXXXXRALGRXXXXXDRRXXQXXXXXXXXXXXXXXXRXASXXXXXXXXXXXXXXXXXXXXXXXXXXXXXXXXXXXXXXXXXXXXXXXXXXXXX

SEQ ID NO: 83 - protein, 77 aa, synthetic construct
  VARIANT 1..17: note = X can be any amino acid residue or can be absent.
  VARIANT 21..22: note = X can be any amino acid residue or can be absent.
  VARIANT 40: note = X can be any amino acid residue or can be absent.
  VARIANT 50..51: note = X can be any amino acid residue or can be absent.
  VARIANT 67: note = X can be any amino acid residue or can be absent.
  VARIANT 72..73: note = X can be any amino acid residue or can be absent.
XXXXXXXXXXXXXXXXXMPVXXGIGIGRGDPLRPAVTRTXRFSGPEGFHXXPGALWLAAAAPLLATXLLLLXXRLAA

SEQ ID NO: 84 - protein, 101 aa, Methylobacterium sp.
MPTAIPIRPAPERCLSWPDTARLLILVARVRILDLEMHTVVRHGSGFADDRLLHLMRRWLAQHEAISALLPGVAEPRHVAEVRAILQVPNSRPEPEDRRAL

SEQ ID NO: 85 - protein, 83 aa, Methylobacterium sp.
MPQRRTIQVTEDGRMNLPADIRRVLGLNGAGRIVLMQDEDGIHLTTAEDPLRRVRELAAPFRRGSGSVVDEFIAERRADSGED

SEQ ID NO: 86 - protein, 48 aa, Methylobacterium sp.
MPLDYALQITAIAFGLSVLGLGGAFIASRVYDRNTRRYDEAAQLHKAD

SEQ ID NO: 87 - protein, 1982 aa, Methylobacterium sp.
VQDGIQITCSVTEHVSLAYHENAVPVIREVVVENTSEQELSDVRVRIESRPAVVQPLTLRIDRIPAGSNHHIELPDVRLDAALLAGFTEASRLELTVIVEDAAGERARHLEELRVLPPSHWGGGRSAPELLAAFVRPNDPAVDVVLRDAATKLGEAGRETGLNGYTTAKKSRAWELAEAIWAAIADRRIAYVLPPASFERAGQKVRGPSDVLERKVGTCLDLSLLYAACLEQAGLNPVLVLTVGHAFVGVWLQDDDFASATVDDMQLLRKRRDLQDLVFVETTLLTPEPPATFKVATTQGGVQVEDEAPAALEIAIDVRRCRRRGIRPMDLGDGKPTGIAPAPTIPLNQTLSAPPSFEEEARAPVDEAPETPVGRVERWKRKLLDLTLRNKLLNFKPGKGSVSLECASPGALEDGLAAGTEYRLKPLSDVLTGSDERSADLYARRHHDDGRRSYLEAALARKEIYTTSTEADLDRRLLDLYRLARNGFEEGGANILFLAVGFLSWTKKEGEAAYRAPLLLVPVTLKRSSVRAGFKLALHDDEVRINPTLLEMLREDFKLRMPELEGDLPRDGSGYDVDGIFRIVRQHVKELRGWEVVPDVVLSAFSFTKYLMWKDLVDRAEVLKRNPVVRHLIDTPKHSYGDGTPFPEPTRLDREHPPETVFAPLSADSSQLSAVLAAAGGKDFVLFGPPGTGKSQTIGNMIAQCLAQGRTVLFVSQKTAALEVVQRRLQEIGLGDYCLEVHSTKAQKSAVLGQLRRAWHERSTPSQGTWDAATSELASLREELNGLVNALHRRRENGLSAYEAFGRVIASGGEAPLVLTWPDHLAHNETTLANLRAACRELRPVLASVGSLVDHPLQGVEATQWSPVWRDDMGAAIRAVEQTLGALRVSGQAFAEAIGLPSLLATYAGTRGLVVLGNYLVRSEARCGAAFLADGAGDLRRAVAARERFQTTKVQLLGRLTGRYRPGILDQNLGALLAEWVAAQGANFLVKGGKLKKVSAQVQPYAEGPLPPDLGPDLTGLIEVARHVKAGCLEELILARLGLPWSNPDCPASEFASAITWAEKVEQLLDILGPLSLGIDGLRDHLVHLVERQGRALADGGRIAQTYAAFAQDRARANEAMKALGVLAGRPDPEEPLAAEADWIERSCTIARRLSSGLSRAQGWCAWQAAAQSALKTGLAPLIDALEDGRIAPDRAEIAFEIAYARWWIDRVVSDDPVLRRFLPARHEDAIQRFRAADARVTELSKQVVRSRLGGGIPGATAFGADPEWGTLSHELTKKTAHMPLRKLFGKMPTALTKLTPCVMMSPLSIAQYLPPDKEPFDVVIFDEASQISPWDAIGALARAKQVVIVGDPEQLPPTNVGDRGVDDIEDGSDVTDQESILDECLAANIPRRNLDWHYRSRHESLIAFSNSRYYGGRLVTFPSPVTDDRAVRLTLVPDGVYKRGSGRVNRPEARAVVADIVRRLRDPSFSEERRSLGVVTFNGEQQRLIENLLDEQRRSYPELEPFFDRDRWHEPVFVKNLENVQGDERDAIIFSVAVGPDQTGRPVSTVSSLNKDGGHRRLNVAITRARRELVVFASMRPEQIDLGRTRARGVRDFKHFLEFAERGARALAEAFAPTGGDVESPFEAAVMAGLEARGWTVHTQIGVSGFRIDLGIVHPDAPGRYLAGVECDGATYHSSATARDRDRLREHVLTDLGWRIRRVWSTEWWMDAEGALTKLDQRLIEDLEADRAKAAAAAAEAPRDVAVEPEAVEQEHDEPTGEPEVTPPVDTGPSEPANDLEPVTDLIPQRLYADQALPVTPPAPKPEVYDDVRAYRIVDLNDLGRSVEPGRFYDASYQQALSAMVDHVLAVEGPIYEELLIKRIARAHDIQRVGPLVREAIADRIDASVARTEDDGRPVLWPRGEEPRASYPHRPASAAIRSHTDTPMPELVGIAMTLPSNASEAERARMIGQRLGLSRIEASARARFERASELARQAAVA

SEQ ID NO: 88 - protein, 210 aa, Methylobacterium sp.
MSVVLYARVSTAEQTLEHQQTQAEAAGFVFDAVVADHGESGRKPLRDRPEGRRLYDMLRTGDVLVVRWINRLGRSYEDVTGVMRELMQRGVIVRTIISNMTFDGATKDPMQRAIRDALIAFMAAAGEAELEATREAQKAGIEHARKQADQTAYRGRKPSYTRDQLTVISGMLGRGAGVSAIAAETGLSRQTIYRVQADPVEAEAALARWA

SEQ ID NO: 89 - protein, 69 aa, Methylobacterium sp.
MLSLDDIAAAAAGEERDALWRSLVEDMEEAAGRRRGGRGLVQADRPADLARALGRDRRVQPSRLARSAS

SEQ ID NO: 90 - protein, 54 aa, Methylobacterium sp.
MPVGIGIGRGDPLRPAVTRTARFSGPEGFHPGALWLAAASPLLATLLLLVRLAA

SEQ ID NO: 91 - DNA, 1471 bp, Methylobacterium sp.
  source 1..1471: mol_type = genomic DNA
ggtgatccagccgcaggttcccctacggctaccttgttacgacttcaccccagtcgctgaccctaccgtggtcgcctgcctccttgcggttggcgcagcgccgtcgggtaagaccaactcccatggtgtgacgggcggtgtgtacaaggcccgggaacgtattcaccgtggcatgctgatccacgattactagcgattccgccttcatgcactcgagttgcagagtgcaatccgaactgagacggcttttggggatttgctccagatcgctccttcgcctcccactgtcaccgccattgtagcacgtgtgtagcccatcccgtaagggccatgaggacttgacgtcatccacaccttcctcgcggcttatcaccggcagtctccctagagtgcccaactgaatgatggcaactaaggacgtgggttgcgctcgttgcgggacttaacccaacatctcacgacacgagctgacgacagccatgcagcacctgtgtgcgcgccaccgaagtggaccccaaatctctctgggtaacacgccatgtcaaaggatggtaaggttctgcgcgttgcttcgaattaaaccacatgctccaccgcttgtgcgggcccccgtcaattcctttgagttttaatcttgcgaccgtactccccaggcggaatgctcaaagcgttagctgcgctactgcggtgcaagcaccccaacagctggcattcatcgtttacggcgtggactaccagggtatctaatcctgtttgctccccacgctttcgcgcctcagcgtcagtaatggtccagttggccgccttcgccaccggtgttcttgcgaatatctacgaatttcacctctacactcgcagttccaccaacctctaccatactcaagcgtcccagtatcgaaggccattctgtggttgagccacaggctttcacccccgacttaaaacgccgcctacgcgccctttacgcccagtgattccgagcaacgctagcccccttcgtattaccgcggctgctggcacgaagttagccggggcttattcctccggtaccgtcattatcgtcccggataaaagagctttacaaccctaaggccttcatcactcacgcggcatggctggatcaggcttgcgcccattgtccaatattccccactgctgcctcccgtaggagtctgggccgtgtctcagtcccagtgtggctgatcatcctctcagaccagctactgatcgtcgccttggtaggccgttaccccaccaactagctaatcagacgcgggccgatcttccggcagtaaacctttccccaaaagggcgtatccggtattagccctagtttcccagggttattccgaaccagaaggcacgttcccacgcgttactcacccgtccgccgctgaccccgaaaggcccgctcgacttgcatgtgttaagcctgccgccagcgttcgctctgagccaggatcaaactctc

SEQ ID NO: 92 - DNA, 1471 bp, Methylobacterium sp.
  source 1..1471: mol_type = genomic DNA
gagtttgatcctggctcagagcgaacgctggcggcaggcttaacacatgcaagtcgagcgggcccttcggggtcagcggcggacgggtgagtaacgcgtgggaacgtgccttctggttcggaataaccctgggaaactagggctaataccggatacgcccttatggggaaaggtttactgccggaagatcggcccgcgtctgattagctagttggtggggtaacggcctaccaaggcgacgatcagtagctggtctgagaggatgatcagccacactgggactgagacacggcccagactcctacgggaggcagcagtggggaatattggacaatgggcgcaagcctgatccagccatgccgcgtgagtgatgaaggccttagggttgtaaagctcttttatccgggacgataatgacggtaccggaggaataagccccggctaacttcgtgccagcagccgcggtaatacgaagggggctagcgttgctcggaatcactgggcgtaaagggcgcgtaggcggcgttttaagtcgggggtgaaagcctgtggctcaaccacagaatggccttcgatactgggacgcttgagtatggtagaggttggtggaactgcgagtgtagaggtgaaattcgtagatattcgcaagaacaccggtggcgaaggcggccaactggaccattactgacgctgaggcgcgaaagcgtggggagcaaacaggattagataccctggtagtccacgccgtaaacgatgaatgccagctgttggggtgcttgcaccgcagtagcgcagctaacgctttgagcattccgcctggggagtacggtcgcaagattaaaactcaaaggaattgacgggggcccgcacaagcggtggagcatgtggtttaattcgaagcaacgcgcagaaccttaccatcctttgacatggcgtgttacccagagagatttggggtccacttcggtggcgcgcacacaggtgctgcatggctgtcgtcagctcgtgtcgtgagatgttgggttaagtcccgcaacgagcgcaacccacgtccttagttgccatcattcagttgggcactctagggagactgccggtgataagccgcgaggaaggtgtggatgacgtcaagtcctcatggcccttacgggatgggctacacacgtgctacaatggcggtgacagtgggacgcgaaggagcgatctggagcaaatccccaaaagccgtctcagttcggattgcactctgcaactcgagtgcatgaaggcggaatcgctagtaatcgtggatcagcatgccacggtgaatacgttcccgggccttgtacacaccgcccgtcacaccatgggagttggtcttacccgacggcgctgcgccaaccgcaaggaggcaggcgaccacggtagggtcagcgactggggtgaagtcgtaacaaggtagccgtaggggaacctgcggctggatcacctc

SEQ ID NO: 93 - DNA, 1473 bp, Methylobacterium sp.
  source 1..1473: mol_type = genomic DNA
ggtgatccagccgcaggttcccctacggctaccttgttacgacttcaccccagtcgctgaccctaccgtggtcgcctgctccccttgcgggtcggcgcagcgccgtcgggtaagaccaactcccatggtgtgacgggcggtgtgtacaaggcccgggaacgtattcaccgtggcatgctgatccacgattactagcgattccgccttcatgcactcgagttgcagagtgcaatccgaactgagacggcttttggagatttgcttgccctcgcgggttcgcgtcccactgtcaccgccattgtagcacgtgtgtagcccatcccgtaagggccatgaggacttgacgtcatccacaccttcctcgcggcttatcaccggcagtctccccagagtgcccaactgaatgatggcaactgaggacgtgggttgcgctcgttgcgggacttaacccaacatctcacgacacgagctgacgacagccatgcagcacctgtgtgcgcgctcccgaaggagaccgtggatctctccacgtaacacgccatgtcaaaggatggtaaggttctgcgcgttgcttcgaattaaaccacatgctccaccgcttgtgcgggcccccgtcaattcctttgagttttaatcttgcgaccgtactccccaggcggaatgctcaaagcgttagctgcgccactgagaggcaagccccccaacggctggcattcatcgtttacggcgtggactaccagggtatctaatcctgtttgctccccacgctttcgcgcctcagcgtcagtgtcggaccagttggccgccttcgccaccggtgttcttgcgaatatctacgaatttcacctctacactcgcagttccaccaacctcttccgaactcaagtctcccagtatcgaaggcaattctgtggttgagccacaggctttcacccccgacttaaaagaccgcctacgcgccctttacgcccagtgattccgagcaacgctagcccccttcgtattaccgcggctgctggcacgaagttagccggggcttattcctccggtaccgtcattatcgtcccggataaaagagctttacaaccctaaggccttcatcactcacgcggcatggctggatcaggcttgcgcccattgtccaatattccccactgctgcctcccgtaggagtctgggccgtgtctcagtcccagtgtggctgatcatcctctcagaccagctactgatcgtcgccttggtaggccgttaccccaccaactagctaatcagacgcgggccgatcttccggcagtaaacctttccccaaaagggcgtatccggtattagctcaagtttccctgagttattccgaaccagaaggcacgttcccacgcgttactcacccgtccgccgctgacaccgaagtgcccgctcgacttgcatgtgttaagcctgccgccagcgttcgctctgagccaggatcaaactctc

SEQ ID NO: 94 - DNA, 1475 bp, Methylobacterium sp.
  source 1..1475: mol_type = genomic DNA
gagtttgatcctggctcagagcgaacgctggcggcaggcttaacacatgcaagtcgagcgggcatcttcgggtgtcagcggcggacgggtgagtaacgcgtgggaacgtgccttctggttcggaataactcagggaaacttgagctaataccggatacgcccttttggggaaaggtttactgccggaagatcggcccgcgtctgattagctagttggtggggtaacggcctaccaaggcgacgatcagtagctggtctgagaggatgatcagccacactgggactgagacacggcccagactcctacgggaggcagcagtggggaatattggacaatgggcgcaagcctgatccagccatgccgcgtgagtgatgaaggccttagggttgtaaagctcttttatccgggacgataatgacggtaccggaggaataagccccggctaacttcgtgccagcagccgcggtaatacgaagggggctagcgttgctcggaatcactgggcgtaaagggcgcgtaggcggtcttttaagtcgggggtgaaagcctgtggctcaaccacagaattgccttcgatactgggagacttgagttcggaagaggttggtggaactgcgagtgtagaggtgaaattcgtagatattcgcaagaacaccggtggcgaaggcggccaactggtccgacactgacgctgaggcgcgaaagcgtggggagcaaacaggattagataccctggtagtccacgccgtaaacgatgaatgccagccgttggggggcttgcctctcagtggcgcagctaacgctttgagcattccgcctggggagtacggtcgcaagattaaaactcaaaggaattgacgggggcccgcacaagcggtggagcatgtggtttaattcgaagcaacgcgcagaaccttaccatcctttgacatggcgtgttacgtggagagatccacggtctccttcgggagcgcgcacacaggtgctgcatggctgtcgtcagctcgtgtcgtgagatgttgggttaagtcccgcaacgagcgcaacccacgtcctcagttgccatcattcagttgggcactctggggagactgccggtgataagccgcgaggaaggtgtggatgacgtcaagtcctcatggcccttacgggatgggctacacacgtgctacaatggcggtgacagtgggacgcgaacccgcgagggcaagcaaatctccaaaagccgtctcagttcggattgcactctgcaactcgagtgcatgaaggcggaatcgctagtaatcgtggatcagcatgccacggtgaatacgttcccgggccttgtacacaccgcccgtcacaccatgggagttggtcttacccgacggcgctgcgccgacccgcaaggggagcaggcgaccacggtagggtcagcgactggggtgaagtcgtaacaaggtagccgtaggggaacctgcggctggatcacctc

SEQ ID NO: 95 - DNA, 1471 bp, Methylobacterium sp.
  source 1..1471: mol_type = genomic DNA
gagtttgatcctggctcagagcgaacgctggcggcaggcttaacacatgcaagtcgagcgggcctttcggggtcagcggcggacgggtgagtaacgcgtgggaacgtgccttctggttcggaataaccctgggaaactagggctaataccggatacgcccttttggggaaaggtttactgccggaagatcggcccgcgtctgattagctagttggtggggtaacggcctaccaaggcgacgatcagtagctggtctgagaggatgatcagccacactgggactgagacacggcccagactcctacgggaggcagcagtggggaatattggacaatgggcgcaagcctgatccagccatgccgcgtgagtgatgaaggccttagggttgtaaagctcttttatccgggacgataatgacggtaccggaggaataagccccggctaacttcgtgccagcagccgcggtaatacgaagggggctagcgttgctcggaatcactgggcgtaaagggcgcgtaggcggcgttttaagtcgggggtgaaagcctgtggctcaaccacagaatggccttcgatactgggacgcttgagtatggtagaggttggtggaactgcgagtgtagaggtgaaattcgtagatattcgcaagaacaccggtggcgaaggcggccaactggaccattactgacgctgaggcgcgaaagcgtggggagcaaacaggattagataccctggtagtccacgccgtaaacgatgaatgccagctgttggggtgcttgcaccgcagtagcgcagctaacgctttgagcattccgcctggggagtacggtcgcaagattaaaactcaaaggaattgacgggggcccgcacaagcggtggagcatgtggtttaattcgaagcaacgcgcagaaccttaccatcctttgacatggcgtgttacccagagagatttggggtccacttcggtggcgcgcacacaggtgctgcatggctgtcgtcagctcgtgtcgtgagatgttgggttaagtcccgcaacgagcgcaacccacgtccttagttgccatcattcagttgggcactctagggagactgccggtgataagccgcgaggaaggtgtggatgacgtcaagtcctcatggcccttacgggatgggctacacacgtgctacaatggcggtgacagtgggacgcgaaggagcgatctggagcaaatccccaaaagccgtctcagttcggattgcactctgcaactcgagtgcatgaaggcggaatcgctagtaatcgtggatcagcatgccacggtgaatacgttcccgggccttgtacacaccgcccgtcacaccatgggagttggtcttacccgacggcgctgcgccaaccgcaaggaggcaggcgaccacggtagggtcagcgactggggtgaagtcgtaacaaggtagccgtaggggaacctgcggctggatcacctc

SEQ ID NO: 96 - DNA, 1471 bp, Methylobacterium sp.; identical to SEQ ID NO: 92
  source 1..1471: mol_type = genomic DNA

SEQ ID NO: 97 - DNA, 1475 bp, Methylobacterium sp.
  source 1..1475: mol_type = genomic DNA
ggtgatccagccgcaggttcccctacggctaccttgttacgacttcaccccagtcgctgaccctaccgtggtcgcctgctccccttgcgggtcggcgcagcgccgtcgggtaagaccaactcccatggtgtgacgggcggtgtgtacaaggcccgggaacgtattcaccgtggcatgctgatccacgattactagcgattccgccttcatgcactcgagttgcagagtgcaatccgaactgagacggcttttggagatttgcttgccctcgcgggttcgcgtcccactgtcaccgccattgtagcacgtgtgtagcccatcccgtaagggccatgaggacttgacgtcatccacaccttcctcgcggcttatcaccggcagtctccccagagtgcccaactaaatgatggcaactgaggacgtgggttgcgctcgttgcgggacttaacccaacatctcacgacacgagctgacgacagccatgcagcacctgtgtgcgcgctcccgaaggagaccgtggatctctccacgtaacacgccatgtcaaaggatggtaaggttctgcgcgttgcttcgaattaaaccacatgctccaccgcttgtgcgggcccccgtcaattcctttgagttttaatcttgcgaccgtactccccaggcggaatgctcaaagcgttagctgcgccactgagaggcaagccccccaacggctggcattcatcgtttacggcgtggactaccagggtatctaatcctgtttgctccccacgctttcgcgcctcagcgtcagtgtcggaccagttggccgccttcgccaccggtgttcttgcgaatatctacgaatttcacctctacactcgcagttccaccaacctcttccgaactcaagtctcccagtatcgaaggcaattctgtggttgagccacaggctttcacccccgacttaaaagaccgcctacgcgccctttacgcccagtgattccgagcaacgctagcccccttcgtattaccgcggctgctggcacgaagttagccggggcttattcctccggtaccgtcattatcgtcccggataaaagagctttacaaccctaaggccttcatcactcacgcggcatggctggatcaggcttgcgcccattgtccaatattccccactgctgcctcccgtaggagtctgggccgtgtctcagtcccagtgtggctgatcatcctctcagaccagctactgatcgtcgccttggtaggccgttaccccaccaactagctaatcagacgcgggccgatcttccggcagtaaacctttccccaaaagggcgtatccggtattagctcaagtttccctgagttattccgaaccagaaggcacgttcccacgcgttactcacccgtccgccgctgacacccgaaagtgcccgctcgacttgcatgtgttaagcctgccgccagcgttcgctctgagccaggatcaaactctc

SEQ ID NO: 98 - DNA, 1473 bp, Methylobacterium sp.
  source 1..1473: mol_type = genomic DNA
ggtgatccagccgcaggttcccctacggctaccttgttacgacttcaccccagtcgctgaccctaccgtggtcgcctgctccccttgcgggtcggcgcagcgccgtcgggtaagaccaactcccatggtgtgacgggcggtgtgtacaaggcccgggaacgtattcaccgtggcatgctgatccacgattactagcgattccgccttcatgcactcgagttgcagagtgcaatccgaactgagacggcttttggagatttgcttgccctcgcgggttcgcgtcccactgtcaccgccattgtagcacgtgtgtagcccatcccgtaagggccatgaggacttgacgtcatccacaccttcctcgcggcttatcaccggcagtctccccagagtgcccaactaaatgatggcaactgaggacgtgggttgcgctcgttgcgggacttaacccaacatctcacgacacgagctgacgacagccatgcagcacctgtgtgcgcgctcccgaaggagaccgtggatctctccacgtaacacgccatgtcaaaggatggtaaggttctgcgcgttgcttcgaattaaaccacatgctccaccgcttgtgcgggcccccgtcaattcctttgagttttaatcttgcgaccgtactccccaggcggaatgctcaaagcgttagctgcgccactgagaggcaagccccccaacggctggcattcatcgtttacggcgtggactaccagggtatctaatcctgtttgctccccacgctttcgcgcctcagcgtcagtgtcggaccagttggccgccttcgccaccggtgttcttgcgaatatctacgaatttcacctctacactcgcagttccaccaacctcttccgaactcaagtctcccagtatcgaaggcaattctgtggttgagccacaggctttcacccccgacttaaaagaccgcctacgcgccctttacgcccagtgattccgagcaacgctagcccccttcgtattaccgcggctgctggcacgaagttagccggggcttattcctccggtaccgtcattatcgtcccggataaaagagctttacaaccctaaggccttcatcactcacgcggcatggctggatcaggcttgcgcccattgtccaatattccccactgctgcctcccgtaggagtctgggccgtgtctcagtcccagtgtggctgatcatcctctcagaccagctactgatcgtcgccttggtaggccgttaccccaccaactagctaatcagacgcgggccgatcttccggcagtaaacctttccccaaaagggcgtatccggtattagctcaagtttccctgagttattccgaaccagaaggcacgttcccacgcgttactcacccgtccgccgctgacaccgaagtgcccgctcgacttgcatgtgttaagcctgccgccagcgttcgctctgagccaggatcaaactctc

SEQ ID NO: 99 - DNA, 1471 bp, Methylobacterium sp.
  source 1..1471: mol_type = genomic DNA
gagtttgatcctggctcagagcgaacgctggcggcaggcttaacacatgcaagtcgagcgggcccttcggggtcagcggcggacgggtgagtaacgcgtgggaacgtgccttctggttcggaataaccctgggaaactagggctaataccggatacgcccttatggggaaaggtttactgccggaagatcggcccgcgtctgattagctagttggtggggtaacggcctaccaaggcgacgatcagtagctggtctgagaggatgatcagccacactgggactgagacacggcccagactcctacgggaggcagcagtggggaatattggacaatgggcgcaagcctgatccagccatgccgcgtgagtgatgaaggccttagggttgtaaagctcttttatccgggacgataatgacggtaccggaggaataagccccggctaacttcgtgccagcagccgcggtaatacgaagggggctagcgttgctcggaatcactgggcgtaaagggcgcgtaggcggcgttttaagtcgggggtgaaagcctgtggctcaaccacagaatggccttcgatactgggacgcttgagtatggtagaggttggtggaactgcgagtgtagaggtgaaattcgtagatattcgcaagaacaccggtggcgaaggcggccaactggaccattactgacgctgaggcgcgaaagcgtggggagcaaacaggattagataccctggtagtccacgccgtaaacgatgaatgccagctgttggggtgcttgcaccgcagtagcgcagctaacgctttgagcattccgcctggggagtacggtcgcaagattaaaactcaaaggaattgacgggggcccgcacaagcggtggagcatgtggtttaattcgaagcaacgcgcagaaccttaccatcctttgacatggcgtgttacccagagagatttggggtccacttcggtggcgcgcacacaggtgctgcatggctgtcgtcagctcgtgtcgtgagatgttgggttaagtcccgcaacgagcgcaacccacgtccttagttgccatcattcagttgggcactctagggagactgccggtgataagccgcgaggaaggtgtggatgacgtcaagtcctcatggcccttacgggatgggctacacacgtgctacaatggcggtgacagtgggacgcgaaggagcgatctggagcaaatccccaaaagccgtctcagttcggattgcactctgcaactcgagtgcatgaaggcggaatcgctagtaatcgtggatcagcatgccacggtgaatacgttcccgggccttgtacacaccgcccgtcacaccatgggagttggtcttacccgacggcgctgcgccaaccgcaagggggcaggcgaccacggtagggtcagcgactggggtgaagtcgtaacaaggtagccgtaggggaacctgcggctggatcacctc

SEQ ID NO: 100 - DNA, 1484 bp, Methylobacterium sp.
  source 1..1484: mol_type = genomic DNA
ggtgatccagccgcaggttcccctacggctaccttgttacgacttcaccccagtcgctgaccctaccgtggtcgcctgcctccagtcgagcaagctcgatttggttggcgcagcgccgtcgggtaagaccaactcccatggtgtgacgggcggtgtgtacaaggcccgggaacgtattcaccgtggcatgctgatccacgattactagcgattccgccttcatgcacgcgagttgcagcgtgcaatccgaactgagacggcttttggagattggctccgggtcaccccttcgcgtcccactgtcaccgccattgtagcacgtgtgtagcccatcccgtaagggccatgaggacttgacgtcatccacaccttcctcgcggcttatcaccggcagtctccctagagtgcccaaccaaatgatggcaactaaggacgtgggttgcgctcgttgcgggacttaacccaacatctcacgacacgagctgacgacagccatgcagcacctgtgtgcgcgcccccgaaggggacctggaatctctcccagtaacacgccatgtcaaaggatggtaaggttctgcgcgttgcttcgaattaaaccacatgctccaccgcttgtgcgggcccccgtcaattcctttgagttttaatcttgcgaccgtactccccaggcggaatgcttaatgcgttagctgcgctactgcggtgcatgcaccccaacagctagcattcatcgtttacggcgtggactaccagggtatctaatcctgtttgctccccacgctttcgcgcctcagcgtcagtaatggtccagttggccgccttcgccaccggtgttcttgcgaatatctacgaatttcacctctacactcgcagttccaccaacctctaccatactcaagcgtcccagtatcgaaggccattctgtggttgagccacaggctttcacccccgacttaaaacgccgcctacgcgccctttacgcccagtgattccgagcaacgctagcccccttcgtattaccgcggctgctggcacgaagttagccggggcttattcctccggtaccgtcattatcgtcccggagaaaagagctttacaaccctaaggccgtcatcactcacgcggcatggctggatcaggcttgcgcccattgtccaatattccccactgctgcctcccgtaggagtctgggccgtgtctcagtcccagtgtggctgatcatcctctcagaccagctactgatcgtcgccttggtaggccgttaccccaccaacaagctaatcagacgcgggccgatcctccggcagtaaacctttctgccaaagcacgtatccggtattagccctagtttcccagggttatcccagaccggagggcacgttcccacgtgttactcacccgtctgccactcaccttgcggtgcgttcgacttgcatgtgttaagcctgccgccagcgttcgctctgagccaggatcaaactctc

SEQ ID NO: 101 - DNA, 1475 bp, Methylobacterium sp.
  source 1..1475: mol_type = genomic DNA
ggtgatccagccgcaggttcccctacggctaccttgttacgacttcaccccagtcgctgaccctaccgtggtcgcctgctccccttgcgggtcggcgcagcgccgtcgggtaagaccaactcccatggtgtgacgggcggtgtgtacaaggcccgggaacgtattcaccgtggcatgctgatccacgattactagcgattccgccttcatgcactcgagttgcagagtgcaatccgaactgagacggcttttggagatttgcttgccctcgcgggttcgcgtcccactgtcaccgccattgtagcacgtgtgtagcccatcccgtaagggccatgaggacttgacgtcatccacaccttcctcgcggcttatcaccggcagtctccccagagtgcccaactgaatgatggcaactgaggacgtgggttgcgctcgttgcgggacttaacccaacatctcacgacacgagctgacgacagccatgcagcacctgtgtgcgcgctcccgaaggagaccgtggatctctccacgtaacacgccatgtcaaaggatggtaaggttctgcgcgttgcttcgaattaaaccacatgctccaccgcttgtgcgggcccccgtcaattcctttgagttttaatcttgcgaccgtactccccaggcggaatgctcaaagcgttagctgcgccactgagaggcaagccccccaacggctggcattcatcgtttacggcgtggactaccagggtatctaatcctgtttgctccccacgctttcgcgcctcagcgtcagtgtcggaccagttggccgccttcgccaccggtgttcttgcgaatatctacgaatttcacctctacactcgcagttccaccaacctcttccgaactcaagtctcccagtatcgaaggcaattctgtggttgagccacaggctttcacccccgacttaaaagaccgcctacgcgccctttacgcccagtgattccgagcaacgctagcccccttcgtattaccgcggctgctggcacgaagttagccggggcttattcctccggtaccgtcattatcgtcccggataaaagagctttacaaccctaaggccttcatcactcacgcggcatggctggatcaggcttgcgcccattgtccaatattccccactgctgcctcccgtaggagtctgggccgtgtctcagtcccagtgtggctgatcatcctctcagaccagctactgatcgtcgccttggtaggccgttaccccaccaactagctaatcagacgcgggccgatcttccggcagtaaacctttccccataagggcgtatccggtattagctcaagtttccctgagttattccgaaccagaaggcacgttcccacgcgttactcacccgtccgccgctgacacccgaaagtgcccgctcgacttgcatgtgttaagcctgccgccagcgttcgctctgagccaggatcaaactctc

SEQ ID NO: 102 - DNA, 1484 bp, Methylobacterium sp.; identical to SEQ ID NO: 100
  source 1..1484: mol_type = genomic DNA

SEQ ID NO: 103 - DNA, 1471 bp, Methylobacterium sp.
  source 1..1471: mol_type = genomic DNA
ggtgatccagccgcaggttcccctacggctaccttgttacgacttcaccccagtcgctgaccctaccgtggtcgcctgcctccttgcggttggcgcagcgccgtcgggtaagaccaactcccatggtgtgacgggcggtgtgtacaaggcccgggaacgtattcaccgtggcatgctgatccacgattactagcgattccgccttcatgcactcgagttgcagagtgcaatccgaactgagacggcttttggggatttgctccagatcgctccttcgcgtcccactgtcaccgccattgtagcacgtgtgtagcccatcccgtaagggccatgaggacttgacgtcatccacaccttcctcgcggcttatcaccggcagtctccctagagtgcccaactgaatgatggcaactaaggacgtgggttgcgctcgttgcgggacttaacccaacatctcacgacacgagctgacgacagccatgcagcacctgtgtgcgcgccaccgaagtggaccccaaatctctctgggtaacacgccatgtcaaaggatggtaaggttctgcgcgttgcttcgaattaaaccacatgctccaccgcttgtgcgggcccccgtcaattcctttgagttttaatcttgcgaccgtactccccaggcggaatgctcaaagcgttagctgcgctactgcggtgcaagcaccccaacagctggcattcatcgtttacggcgtggactaccagggtatctaatcctgtttgctccccacgctttcgcgcctcagcgtcagtaatggtccagttggccgccttcgccaccggtgttcttgcgaatatctacgaatttcacctctacactcgcagttccaccaacctctaccatactcaagcgtcccagtatcgaaggccattctgtggttgagccacaggctttcacccccgacttaaaacgccgcctacgcgccctttacgcccagtgattccgagcaacgctagcccccttcgtattaccgcggctgctggcacgaagttagccggggcttattcctccggtaccgtcattatcgtcccggataaaagagctttacaaccctaaggccttcatcactcacgcggcatggctggatcaggcttgcgcccattgtccaatattccccactgctgcctcccgtaggagtctgggccgtgtctcagtcccagtgtggctgatcatcctctcagaccagctactgatcgtcgccttggtaggccgttaccccaccaactagctaatcagacgcgggccgatcttccggcagtaaacctttccccataagggcgtatccggtattagccctagtttcccagggttattccgaaccggaaggcacgttcccacgcgttactcacccgtccgccgctgaccccgaaaggtccgctcgacttgcatgtgttaagcctgccgccagcgttcgctctgagccaggatcaaactctc

SEQ ID NO: 104 - DNA, 1475 bp, Methylobacterium sp.; identical to SEQ ID NO: 94
  source 1..1475: mol_type = genomic DNA

SEQ ID NO: 105 - DNA, 1471 bp, Methylobacterium sp.
  source 1..1471: mol_type = genomic DNA
ggtgatccagccgcaggttcccctacggctaccttgttacgacttcaccccagtcgctgaccctaccgtggtcgcctgcctccttgcggttggcgcagcgccgtcgggtaagaccaactcccatggtgtgacgggcggtgtgtacaaggcccgggaacgtattcaccgtggcatgctgatccacgattactagcgattccgccttcatgcactcgagttgcagagtgcaatccgaactgagacggcttttggggatttgctccagatcgctccttcgcgtcccactgtcaccgccattgtagcacgtgtgtagcccatcccgtaagggccatgaggacttgacgtcatccacaccttcctcgcggcttatcaccggcagtctccctagagtgcccaactgaatgatggcaactaaggacgtgggttgcgctcgttgcgggacttaacccaacatctcacgacacgagctgacgacagccatgcagcacctgtgtgcgcgccaccgaagtggaccccaaatctctctgggtaacacgccatgtcaaaggatggtaaggttctgcgcgttgcttcgaattaaaccacatgctccaccgcttgtgcgggcccccgtcaattcctttgagttttaatcttgcgaccgtactccccaggcggaatgctcaaagcgttagctgcgctactgcggtgcaagcaccccaacagctggcattcatcgtttacggcgtggactaccagggtatctaatcctgtttgctccccacgctttcgcgcctcagcgtcagtaatggtccagttggccgccttcgccaccggtgttcttgcgaatatctacgaatttcacctctacactcgcagttccaccaacctctaccatactcaagcgtcccagtatcgaaggccattctgtggttgagccacaggctttcacccccgacttaaaacgccgcctacgcgccctttacgcccagtgattccgagcaacgctagcccccttcgtattaccgcggctgctggcacgaagttagccggggcttattcctccggtaccgtcattatcgtcccggataaaagagctttacaaccctaaggccttcatcactcacgcggcatggctggatcaggcttgcgcccattgtccaatattccccactgctgcctcccgtaggagtctgggccgtgtctcagtcccagtgtggctgatcatcctctcagaccagctactgatcgtcgccttggtaggccgttaccccaccaactagctaatcagacgcgggccgatcttccggcagtaaacctttccccaaaagggcgtatccggtattagccctagtttcccagggttattccgaaccagaaggcacgttcccacgcgttactcacccgtccgccgctgaccccgaagggcccgctcgacttgcatgtgttaagcctgccgccagcgttcgctctgagccaggatcaaactctc

SEQ ID NO: 106 - DNA, 1471 bp, Methylobacterium sp.
  source 1..1471: mol_type = genomic DNA
gagtttgatcctggctcagagcgaacgctggcggcaggcttaacacatgcaagtcgagcgggcccttcggggtcagcggcggacgggtgagtaacgcgtgggaacgtgccttctggttcggaataaccctgggaaactagggctaataccggatacgcccttttggggaaaggtttactgccggaagatcggcccgcgtctgattagctagttggtggggtaacggcctaccaaggcgacgatcagtagctggtctgagaggatgatcagccacactgggactgagacacggcccagactcctacgggaggcagcagtggggaatattggacaatgggcgcaagcctgatccagccatgccgcgtgagtgatgaaggccttagggttgtaaagctcttttatccgggacgataatgacggtaccggaggaataagccccggctaacttcgtgccagcagccgcggtaatacgaagggggctagcgttgctcggaatcactgggcgtaaagggcgcgtaggcggcgttttaagtcgggggtgaaagcctgtggctcaaccacagaatggccttcgatactgggacgcttgagtatggtagaggttggtggaactgcgagtgtagaggtgaaattcgtagatattcgcaagaacaccggtggcgaaggcggccaactggaccattactgacgctgaggcgcgaaagcgtggggagcaaacaggattagataccctggtagtccacgccgtaaacgatgaatgccagctgttggggtgcttgcaccgcagtagcgcagctaacgctttgagcattccgcctggggagtacggtcgcaagattaaaactcaaaggaattgacgggggcccgcacaagcggtggagcatgtggtttaattcgaagcaacgcgcagaaccttaccatcctttgacatggcgtgttacccagagagatttggggtccacttcggtggcgcgcacacaggtgctgcatggctgtcgtcagctcgtgtcgtgagatgttgggttaagtcccgcaacgagcgcaacccacgtccttagttgccatcattcagttgggcactctagggagactgccggtgataagccgcgaggaaggtgtggatgacgtcaagtcctcatggcccttacgggatgggctacacacgtgctacaatggcggtgacagtgggacgcgaaggagcgatctggagcaaatccccaaaagccgtctcagttcggattgcactctgcaactcgagtgcatgaaggcggaatcgctagtaatcgtggatcagcatgccacggtgaatacgttcccgggccttgtacacaccgcccgtcacaccatgggagttggtcttacccgacggcgctgcgccaaccgcaaggaggcaggcgaccacggtagggtcagcgactggggtgaagtcgtaacaaggtagccgtaggggaacctgcggctggatcacctc

SEQ ID NO: 107 - DNA, 1471 bp, Methylobacterium sp.
  source 1..1471: mol_type = genomic DNA
ggtgatccagccgcaggttcccctacggctaccttgttacgacttcaccccagtcgctgaccctaccgtggtcgcctgcccccttgcggttagcgcagcgccgtcgggtaagaccaactcccatggtgtgacgggcggtgtgtacaaggcccgggaacgtattcaccgtggcatgctgatccacgattactagcgattccaccttcatgcactcgagttgcagagtgcaatccgaactgagacggcttttggggatttgctcaacctcgcggtctcgcgtcccactgtcaccgccattgtagcacgtgtgtagcccatcccgtaagggccatgaggacttgacgtcatccacaccttcctcgcggcttatcaccggcagtctccccagagtgcccaactgaatgatggcaactgaggacgtgggttgcgctcgttgcgggacttaacccaacatctcacgacacgagctgacgacagccatgcagcacctgtgtgcgcgcctccgaagaggacctggaatctctcccagtaacacgccatgtcaaaggatggtaaggttctgcgcgttgcttcgaattaaaccacatgctccaccgcttgtgcgggcccccgtcaattcctttgagttttaatcttgcgaccgtactccccaggcggaatgcttaatgcgttagctgcgctactgagatgcatgcaccccaacagctagcattcatcgtttacggcgtggactaccagggtatctaatcctgtttgctccccacgctttcgcgcctcagcgtcagtgctggtccagttggccgccttcgccaccggtgttcttgcgaatatctacgaatttcacctctacactcgcagttccaccaacctctaccagactcaagcgtcccagtatcgaaggccattctgtggttgagccacaggctttcacccccgacttaaaacgccgcctacgcgccctttacgcccagtgattccgagcaacgctagcccccttcgtattaccgcggctgctggcacgaagttagccggggcttattcctccggtaccgtcattatcgtcccggataaaagagctttacaaccctaaggccttcatcactcacgcggcatggctggatcaggcttgcgcccattgtccaatattccccactgctgcctcccgtaggagtctgggccgtgtctcagtcccagtgtggctgatcatcctctcagaccagctactgatcgtcgccttggtaggccgttaccccaccaactagctaatcagacgcgggccgatccttcggcagtaaacctttccccaaaagggcgtatccggtattagctccagtttcccagagttgtcccgaaccaaagggtacgttcccacgtgttactcacccgtctgccactagctccgaagagcccgttcgacttgcatgtgttaagcctgccgccagcgttcgctctgagccaggatcaaactctc

SEQ ID NO: 108 - DNA, 1471 bp, Methylobacterium sp.
  source 1..1471: mol_type = genomic DNA
agagtttgatcctggctcagagcgaacgctggcggcaggcttaacacatgcaagtcgagcggacctttcggggtcagcggcggacgggtgagtaacgcgtgggaacgtgccttccggttcggaataaccctgggaaactagggctaataccggatacgcccttatggggaaaggtttactgccggaagatcggcccgcgtctgattagctagttggtggggtaacggcctaccaaggcgacgatcagtagctggtctgagaggatgatcagccacactgggactgagacacggcccagactcctacgggaggcagcagtggggaatattggacaatgggcgcaagcctgatccagccatgccgcgtgagtgatgaaggccttagggttgtaaagctcttttatccgggacgataatgacggtaccggaggaataagccccggctaacttcgtgccagcagccgcggtaatacgaagggggctagcgttgctcggaatcactgggcgtaaagggcgcgtaggcggcgttttaagtcgggggtgaaagcctgtggctcaaccacagaatggccttcgatactgggacgcttgagtatggtagaggttggtggaactgcgagtgtagaggtgaaattcgtagatattcgcaagaacaccggtggcgaaggcggccaactggaccattactgacgctgaggcgcgaaagcgtggggagcaaacaggattagataccctggtagtccacgccgtaaacgatgaatgccagctgttggggtgcttgcaccgcagtagcgcagctaacgctttgagcattccgcctggggagtacggtcgcaagattaaaactcaaaggaattgacgggggcccgcacaagcggtggagcatgtggtttaattcgaagcaacgcgcagaaccttaccatcctttgacatggcgtgttacccagagagatttggggtccacttcggtggcgcgcacacaggtgctgcatggctgtcgtcagctcgtgtcgtgagatgttgggttaagtcccgcaacgagcgcaacccacgtccttagttgccatcattcagttgggcactctagggagactgccggtgataagccgcgaggaaggtgtggatgacgtcaagtcctcatggcccttacgggatgggctacacacgtgctacaatggcggtgacagtgggacgcgaaggagcgatctggagcaaatccccaaaagccgtctcagttcggattgcactctgcaactcgagtgcatgaaggcggaatcgctagtaatcgtggatcagcatgccacggtgaatacgttcccgggccttgtacacaccgcccgtcacaccatgggagttggtcttacccgacggcgctgcgccaaccgcaaggaggcaggcgaccacggtagggtcagcgactggggtgaagtcgtaacaaggtagccgtaggggaacctgcggctggatcacct

SEQ ID NO: 109 - DNA, 1471 bp, Methylobacterium sp.
  source 1..1471: mol_type = genomic DNA
agagtttgatcctggctcagagcgaacgctggcggcaggcttaacacatgcaagtcgagcgggcccttcggggtcagcggcggacgggtgagtaacgcgtgggaacgtgccttctggttcggaataaccctgggaaactagggctaataccggatacgcccttttggggaaaggtttactgccggaagatcggcccgcgtctgattagctagttggtggggtaacggcctaccaaggcgacgatcagtagctggtctgagaggatgatcagccacactgggactgagacacggcccagactcctacgggaggcagcagtggggaatattggacaatgggcgcaagcctgatccagccatgccgcgtgagtgatgaaggccttagggttgtaaagctcttttatccgggacgataatgacggtaccggaggaataagccccggctaacttcgtgccagcagccgcggtaatacgaagggggctagcgttgctcggaatcactgggcgtaaagggcgcgtaggcggcgttttaagtcgggggtgaaagcctgtggctcaaccacagaatggccttcgatactgggacgcttgagtatggtagaggttggtggaactgcgagtgtagaggtgaaattcgtagatattcgcaagaacaccggtggcgaaggcggccaactggaccattactgacgctgaggcgcgaaagcgtggggagcaaacaggattagataccctggtagtccacgccgtaaacgatgaatgccagctgttggggtgcttgcaccgcagtagcgcagctaacgctttgagcattccgcctggggagtacggtcgcaagattaaaactcaaaggaattgacgggggcccgcacaagcggtggagcatgtggtttaattcgaagcaacgcgcagaaccttaccatcctttgacatggcgtgttacccagagagatttggggtccacttcggtggcgcgcacacaggtgctgcatggctgtcgtcagctcgtgtcgtgagatgttgggttaagtcccgcaacgagcgcaacccacgtccttagttgccatcattcagttgggcactctagggagactgccggtgataagccgcgaggaaggtgtggatgacgtcaagtcctcatggcccttacgggatgggctacacacgtgctacaatggcggtgacagtgggacgcgaaggagcgatctggagcaaatccccaaaagccgtctcagttcggattgcactctgcaactcgagtgcatgaaggcggaatcgctagtaatcgtggatcagcatgccacggtgaatacgttcccgggccttgtacacaccgcccgtcacaccatgggagttggtcttacccgacggcgctgcgccaaccgcaaggaggcaggcgaccacggtagggtcagcgactggggtgaagtcgtaacaaggtagccgtaggggaacctgcggctggatcacct

SEQ ID NO: 110 - DNA, 1471 bp, Methylobacterium sp.; identical to SEQ ID NO: 108
  source 1..1471: mol_type = genomic DNA

SEQ ID NO: 111 - DNA, 1473 bp, Methylobacterium sp.
  source 1..1473: mol_type = genomic DNA
agagtttgatcctggctcagagcgaacgctggcggcaggcttaacacatgcaagtcgagcgggcacttcggtgtcagcggcggacgggtgagtaacgcgtgggaacgtgccttctggttcggaataactcagggaaacttgagctaataccggatacgcccttttggggaaaggtttactgccggaagatcggcccgcgtctgattagctagttggtggggtaacggcctaccaaggcgacgatcagtagctggtctgagaggatgatcagccacactgggactgagacacggcccagactcctacgggaggcagcagtggggaatattggacaatgggcgcaagcctgatccagccatgccgcgtgagtgatgaaggccttagggttgtaaagctcttttatccgggacgataatgacggtaccggaggaataagccccggctaacttcgtgccagcagccgcggtaatacgaagggggctagcgttgctcggaatcactgggcgtaaagggcgcgtaggcggtcttttaagtcgggggtgaaagcctgtggctcaaccacagaattgccttcgatactgggagacttgagttcggaagaggttggtggaactgcgagtgtagaggtgaaattcgtagatattcgcaagaacaccggtggcgaaggcggccaactggtccgacactgacgctgaggcgcgaaagcgtggggagcaaacaggattagataccctggtagtccacgccgtaaacgatgaatgccagccgttggggggcttgcctctcagtggcgcagctaacgctttgagcattccgcctggggagtacggtcgcaagattaaaactcaaaggaattgacgggggcccgcacaagcggtggagcatgtggtttaattcgaagcaacgcgcagaaccttaccatcctttgacatggcgtgttacgtggagagatccacggtctccttcgggagcgcgcacacaggtgctgcatggctgtcgtcagctcgtgtcgtgagatgttgggttaagtcccgcaacgagcgcaacccacgtcctcagttgccatcattcagttgggcactctggggagactgccggtgataagccgcgaggaaggtgtggatgacgtcaagtcctcatggcccttacgggatgggctacacacgtgctacaatggcggtgacagtgggacgcgaacccgcgagggcaagcaaatctccaaaagccgtctcagttcggattgcactctgcaactcgagtgcatgaaggcggaatcgctagtaatcgtggatcagcatgccacggtgaatacgttcccgggccttgtacacaccgcccgtcacaccatgggagttggtcttacccgacggcgctgcgccgacccgcaaggggagcaggcgaccacggtagggtcagcgactggggtgaagtcgtaacaaggtagccgtaggggaacctgcggctggatcacct

SEQ ID NO: 112 - DNA, 1475 bp, Methylobacterium sp.
  source 1..1475: mol_type = genomic DNA
agagtttgatcctggctcagagcgaacgctggcggcaggcttaacacatgcaagtcgagcgggcatcttcgggtgtcagcggcggacgggtgagtaacgcgtgggaacgtgccttctggttcggaataactcagggaaacttgagctaataccggatacgcccttatggggaaaggtttactgccggaagatcggcccgcgtctgattagctagttggtggggtaacggcctaccaaggcgacgatcagtagctggtctgagaggatgatcagccacactgggactgagacacggcccagactcctacgggaggcagcagtggggaatattggacaatgggcgcaagcctgatccagccatgccgcgtgagtgatgaaggccttagggttgtaaagctcttttatccgggacgataatgacggtaccggaggaataagccccggctaacttcgtgccagcagccgcggtaatacgaagggggctagcgttgctcggaatcactgggcgtaaagggcgcgtaggcggtcttttaagtcgggggtgaaagcctgtggctcaaccacagaattgccttcgatactgggagacttgagttcggaagaggttggtggaactgcgagtgtagaggtgaaattcgtagatattcgcaagaacaccggtggcgaaggcggccaactggtccgacactgacgctgaggcgcgaaagcgtggggagcaaacaggattagataccctggtagtccacgccgtaaacgatgaatgccagccgttggggggcttgcctctcagtggcgcagctaacgctttgagcattccgcctggggagtacggtcgcaagattaaaactcaaaggaattgacgggggcccgcacaagcggtggagcatgtggtttaattcgaagcaacgcgcagaaccttaccatcctttgacatggcgtgttacgtggagagatccacggtctccttcgggagcgcgcacacaggtgctgcatggctgtcgtcagctcgtgtcgtgagatgttgggttaagtcccgcaacgagcgcaacccacgtcctcagttgccatcattcagttgggcactctggggagactgccggtgataagccgcgaggaaggtgtggatgacgtcaagtcctcatggcccttacgggatgggctacacacgtgctacaatggcggtgacagtgggacgcgaacccgcgagggcaagcaaatctccaaaagccgtctcagttcggattgcactctgcaactcgagtgcatgaaggcggaatcgctagtaatcgtggatcagcatgccacggtgaatacgttcccgggccttgtacacaccgcccgtcacaccatgggagttggtcttacccgacggcgctgcgccgacccgcaaggggagcaggcgaccacggtagggtcagcgactggggtgaagtcgtaacaaggtagccgtaggggaacctgcggctggatcacct

SEQ ID NO: 113 - DNA, 1470 bp, Methylobacterium sp.
  source 1..1470: mol_type = genomic DNA
gagtttgatcctggctcagagcgaacgctggcggcaggcttaacacatgcaagtcgagcggacctttcggggtcagcggcggacgggtgagtaacgcgtgggaacgtgccttccggttcggaataaccctgggaaactagggctaataccggatacgcccttatggggaaaggtttactgccggaagatcggcccgcgtctgattagctagttggtggggtaacggcctaccaaggcgacgatcagtagctggtctgagaggatgatcagccacactgggactgagacacggcccagactcctacgggaggcagcagtggggaatattggacaatgggcgcaagcctgatccagccatgccgcgtgagtgatgaaggccttagggttgtaaagctcttttatccgggacgataatgacggtaccggaggaataagccccggctaacttcgtgccagcagccgcggtaatacgaagggggctagcgttgctcggaatcactgggcgtaaagggcgcgtaggcggcgttttaagtcgggggtgaaagcctgtggctcaaccacagaatggccttcgatactgggacgcttgagtatggtagaggttggtggaactgcgagtgtagaggtgaaattcgtagatattcgcaagaacaccggtggcgaaggcggccaactggaccattactgacgctgaggcgcgaaagcgtggggagcaaacaggattagataccctggtagtccacgccgtaaacgatgaatgccagctgttggggtgcttgcaccgcagtagcgcagctaacgctttgagcattccgcctggggagtacggtcgcaagattaaaactcaaaggaattgacgggggcccgcacaagcggtggagcatgtggtttaattcgaagcaacgcgcagaaccttaccatcctttgacatggcgtgttacccagagagatttggggtccacttcggtggcgcgcacacaggtgctgcatggctgtcgtcagctcgtgtcgtgagatgttgggttaagtcccgcaacgagcgcaacccacgtccttagttgccatcattcagttgggcactctagggagactgccggtgataagccgcgaggaaggtgtggatgacgtcaagtcctcatggcccttacgggatgggctacacacgtgctacaatggcggtgacagtgggacgcgaaggagcgatctggagcaaatccccaaaagccgtctcagttcggattgcactctgcaactcgagtgcatgaaggcggaatcgctagtaatcgtggatcagcatgccacggtgaatacgttcccgggccttgtacacaccgcccgtcacaccatgggagttggtcttacccgacggcgctgcgccaaccgcaaggaggcaggcgaccacggtagggtcagcgactggggtgaagtcgtaacaaggtagccgtaggggaacctgcggctggatcacct

SEQ ID NO: 114 - DNA, 1471 bp, Methylobacterium sp.
  source 1..1471: mol_type = genomic DNA
gagtttgatcctggctcagagcgaacgctggcggcaggcttaacacatgcaagtcgagcgggcctttcggggtcagcggcggacgggtgagtaacgcgtgggaacgtgccttctggttcggaataaccctgggaaactagggctaataccggatacgccctttttggggaaaggtttactgccggaagatcggcccgcgtctgattagctagttggtggggtaacggcctaccaaggcgacgatcagtagctggtctgagaggatgatcagccacactgggactgagacacggcccagactcctacgggaggcagcagtggggaatattggacaatgggcgcaagcctgatccagccatgccgcgtgagtgatgaaggccttagggttgtaaagctcttttatccgggacgataatgacggtaccggaggaataagccccggctaacttcgtgccagcagccgcggtaatacgaagggggctagcgttgctcggaatcactgggcgtaaagggcgcgtaggcggcgttttaagtcgggggtgaaagcctgtggctcaaccacagaatggccttcgatactgggacgcttgagtatggtagaggttggtggaactgcgagtgtagaggtgaaattcgtagatattcgcaagaacaccggtggcgaaggcggccaactggaccattactgacgctgaggcgcgaaagcgtggggagcaaacaggattagataccctggtagtccacgccgtaaacgatgaatgccagctgttggggtgcttgcaccgcagtagcgcagctaacgctttgagcattccgcctggggagtacggtcgcaagattaaaactcaaaggaattgacgggggcccgcacaagcggtggagcatgtggtttaattcgaagcaacgcgcagaaccttaccatcctttgacatggcgtgttacccagagagatttggggtccacttcggtggcgcgcacacaggtgctgcatggctgtcgtcagctcgtgtcgtgagatgttgggttaagtcccgcaacgagcgcaacccacgtccttagttgccatcattcagttgggcactctagggagactgccggtgataagccgcgaggaaggtgtggatgacgtcaagtcctcatggcccttacgggatgggctacacacgtgctacaatggcggtgacagtgggacgcgaaggagcgatctggagcaaatccccaaaagccgtctcagttcggattgcactctgcaactcgagtgcatgaaggcggaatcgctagtaatcgtggatcagcatgccacggtgaatacgttcccgggccttgtacacaccgcccgtcacaccatgggagttggtcttacccgacggcgctgcgccaaccgcgaggaggcaggcgaccacggtagggtcagcgactggggtgaagtcgtaacaaggtagccgtaggggaacctgcggctggatcacct

SEQ ID NO: 115 - DNA, 1470 bp, Methylobacterium sp.
  source 1..1470: mol_type = genomic DNA
gagtttgatcctggctcagagcgaacgctggcggcaggcttaacacatgcaagtcgagcgggcacttcggtgtcagcggcggacgggtgagtaacgcgtgggaacgtgccctctggttcggaataactcagggaaacttgagctaataccggatacgcccttttggggaaaggcttgctgccggaggatcggcccgcgtctgattagctagttggtgaggtaacggctcaccaaggcgacgatcagtagctggtctgagaggatgatcagccacactgggactgagacacggcccagactcctacgggaggcagcagtggggaatattggacaatgggcgcaagcctgatccagccatgccgcgtgagtgatgaaggccttagggttgtaaagctcttttatccgggacgataatgacggtaccggaggaataagccccggctaacttcgtgccagcagccgcggtaatacgaagggggctagcgttgctcggaatcactgggcgtaaagggcgcgtaggcggcgttttaagtcgggggtgaaagcctgtggctcaaccacagaatggccttcgatactgggacgcttgagtatggtagaggttggtggaactgcgagtgtagaggtgaaattcgtagatattcgcaagaacaccggtggcgaaggcggccaactggaccattactgacgctgaggcgcgaaagcgtggggagcaaacaggattagataccctggtagtccacgccgtaaacgatgaatgccagctgttggggtgcttgcacctcagtagcgcagctaacgctttgagcattccgcctggggagtacggtcgcaagattaaaactcaaaggaattgacgggggcccgcacaagcggtggagcatgtggtttaattcgaagcaacgcgcagaaccttaccatcctttgacatggcgtgttatggggagagattcccagtccacttcggtggcgcgcacacaggtgctgcatggctgtcgtcagctcgtgtcgtgagatgttgggttaagtcccgcaacgagcgcaacccacgtccttagttgccatcattcagttgggcactctagggagactgccggtgataagccgcgaggaaggtgtggatgacgtcaagtcctcatggcccttacgggatgggctacacacgtgctacaatggcggtgacagtgggacgcgaaggggcgacctggagcaaatccccaaaagccgtctcagttcggattgcactctgcaactcgggtgcatgaaggcggaatcgctagtaatcgtggatcagcatgccacggtgaatacgttcccgggccttgtacacaccgcccgtcacaccatgggagttggtcttacccgacggcgctgcgccaaccgcaaggaggcaggcgaccacggtagggtcagcgactggggtgaagtcgtaacaaggtagccgtaggggaacctgcggctggatcacct

SEQ ID NO: 116 - DNA, 1470 bp, Methylobacterium sp.; identical to SEQ ID NO: 113
  source 1..1470: mol_type = genomic DNA

SEQ ID NO: 117 - DNA, 1474 bp, Methylobacterium sp.
  source 1..1474: mol_type = genomic DNA
ggtgatccagccgcaggttcccctacggctaccttgttacgacttcaccccagtcgctgaccctaccgtggtcgcctgctccccttgcgggtcggcgcagcgccgtcgggtaagaccaactcccatggtgtgacgggcggtgtgtacaaggcccgggaacgtattcaccgtggcatgctgatccacgattactagcgattccgccttcatgcactcgagttgcagagtgcaatccgaactgagacggcttttggagatttgcttgccctcgcgggttcgcgtcccactgtcaccgccattgtagcacgtgtgtagcccatcccgtaagggccatgaggacttgacgtcatccacaccttcctcgcggcttatcaccggcagtctccccagagtgcccaactgaatgatggcaactgaggacgtgggttgcgctcgttgcgggacttaacccaacatctcacgacacgagctgacgacagccatgcagcacctgtgtgcgcgctcccgaaggagaccgtggatctctccacgtaacacgccatgtcaaaggatggtaaggttctgcgcgttgcttcgaattaaaccacatgctccaccgcttgtgcgggcccccgtcaattcctttgagttttaatcttgcgaccgtactccccaggcggaatgctcaaagcgttagctgcgccactgagaggcaagccccccaacggctggcattcatcgtttacggcgtggactaccagggtatctaatcctgtttgctccccacgctttcgcgcctcagcgtcagtgtcggaccagttggccgccttcgccaccggtgttcttgcgaatatctacgaatttcacctctacactcgcagttccaccaacctcttccgaactcaagtctcccagtatcgaaggcaattctgtggttgagccacaggctttcacccccgacttaaaagaccgcctacgcgccctttacgcccagtgattccgagcaacgctagcccccttcgtattaccgcggctgctggcacgaagttagccggggcttattcctccggtaccgtcattatcgtcccggataaaagagctttacaaccctaaggccttcatcactcacgcggcatggctggatcaggcttgcgcccattgtccaatattccccactgctgcctcccgtaggagtctgggccgtgtctcagtcccagtgtggctgatcatcctctcagaccagctactgatcgtcgccttggtaggccgttaccccaccaactagctaatcagacgcgggccgatcttccggcagtaaacctttccccaaaagggcgtatccggtattagctcaagtttccctgagttattccgaaccagaaggcacgttcccacgcgttactcacccgtccgccgctgacacccgaaagtgcccgctcgacttgcatgtgttaagcctgccgccagcgttcgctctgagccaggatcaaactct

SEQ ID NO: 118 - DNA, 1472 bp, Methylobacterium sp.
  source 1..1472: mol_type = genomic DNA
gagtttgatcctggctcagagcgaacgctggcggcaggcttaacacatgcaagtcgagcgggcactttcgggtgtcagcggcggacgggtgagtaacgcgtgggaacgtgccctctggttcggaataactcagggaaacttgagctaataccggatacgcccttttggggaaaggcttgctgccggaggatcggcccgcgtctgattagctagttggtgaggtaacggctcaccaaggcgacgatcagtagctggtctgagaggatgatcagccacactgggactgagacacggcccagactcctacgggaggcagcagtggggaatattggacaatgggcgcaagcctgatccagccatgccgcgtgagtgatgaaggccttagggttgtaaagctcttttatccgggacgataatgacggtaccggaggaataagccccggctaacttcgtgccagcagccgcggtaatacgaagggggctagcgttgctcggaatcactgggcgtaaagggcgcgtaggcggcgttttaagtcgggggtgaaagcctgtggctcaaccacagaatggccttcgatactggaacgcttgagtatggtagaggttggtggaactgcgagtgtagaggtgaaattcgtagatattcgcaagaacaccggtggcgaaggcggccaactggaccattactgacgctgaggcgcgaaagcgtggggagcaaacaggattagataccctggtagtccacgccgtaaacgatgaatgccagctgttggggtgcttgcacctcagtagcgcagctaacgctttgagcattccgcctggggagtacggtcgcaagattaaaactcaaaggaattgacgggggcccgcacaagcggtggagcatgtggtttaattcgaagcaacgcgcagaaccttaccatcctttgacatggcgtgttatggggagagattcccagtcctcttcggaggcgcgcacacaggtgctgcatggctgtcgtcagctcgtgtcgtgagatgttgggttaagtcccgcaacgagcgcaacccacgtccttagttgccatcattcagttgggcactctagggagactgccggtgataagccgcgaggaaggtgtggatgacgtcaagtcctcatggcccttacgggatgggctacacacgtgctacaatggcggtgacagtgggacgcgaaagggcgacctggagcaaatccccaaaagccgtctcagttcggattgcactctgcaactcgggtgcatgaaggcggaatcgctagtaatcgtggatcagcatgccacggtgaatacgttcccgggccttgtacacaccgcccgtcacaccatgggagttggtcttacccgacggcgctgcgccaaccgcaaggaggcaggcgaccacggtagggtcagcgactggggtgaagtcgtaacaaggtagccgtaggggaacctgcggctggatcacct

SEQ ID NO: 119 - DNA, 1470 bp, Methylobacterium sp.
  source 1..1470: mol_type = genomic DNA
ggtgatccagccgcaggttcccctacggctaccttgttacgacttcaccccagtcgctgaccctaccgtggtcgcctgcctccttgcggttggcgcagcgccgtcgggtaagaccaactcccatggtgtgacgggcggtgtgtacaaggcccgggaacgtattcaccgtggcatgctgatccacgattactagcgattccgccttcatgcactcgagttgcagagtgcaatctgaactgagacggtttttggggatttgctcaacctcgcggtttcgcgtcccactgtcaccgccattgtagcacgtgtgtagcccatcccgtaagggccatgaggacttgacgtcatccacaccttcctcgcggcttatcaccggcagtctccctagagtgcccaactgaatgatggcaactaaggacgtgggttgcgctcgttgcgggacttaacccaacatctcacgacacgagctgacgacagccatgcagcacctgtgttcgcgtccccgaagggaaccccggatctctccggatggcacgacatgtcaagggatggtaaggttctgcgcgttgcttcgaattaaaccacatgctccaccgcttgtgcgggcccccgtcaattcctttgagttttaatcttgcgaccgtactccccaggcggaatgcttaaagcgttagctgcgctactgaggtgcaagcaccccaacagctagcattcatcgtttacggcgtggactaccagggtatctaatcctgtttgctccccacgctttcgcgcctcagcgtcagtgttggtccagttggccgccttcgccactggtgttcttgcgaatatctacgaatttcacctctacactcgcagttccaccaacctctaccaaactcaagccaaacagtatcgaaggcaattctgtggttgagccacaggctttcacccccgacttgaatggccgcctacgcgccctttacgcccagtgattccgagcaacgctagcccccttcgtattaccgcggctgctggcacgaagttagccggggcttattcttccggtaccgtcattatcgtcccggacaaaagagctttacaaccctaaggccttcatcactcacgcggcatggctggatcaggcttgcgcccattgtccaatattccccactgctgcctcccgtaggagtctgggccgtgtctcagtcccagtgtggctgatcatcctctcagaccagctactgatcgtcgccttggtaggccattaccccaccaactagctaatcagacgcgggccgatccttcggcagcaagcctttccccaaaagggcgtatccggtattagcgctagtttcccagcgttattccgaaccaaagggtacgttcccacgtgttactcacccgtctgccgctgaccccgaagggcccgctcgacttgcatgtgttaagcctgccgccagcgttcgctctgagccaggatcaaactct

SEQ ID NO: 120 - DNA, 1472 bp, Methylobacterium sp.
  source 1..1472: mol_type = genomic DNA
ggtgatccagccgcaggttcccctacggctaccttgttacgacttcaccccagtcgctgaccctaccgtggtcgcctgctccccttgcgggtcggcgcagcgccgtcgggtaagaccaactcccatggtgtgacgggcggtgtgtacaaggcccgggaacgtattcaccgtggcatgctgatccacgattactagcgattccgccttcatgcactcgagttgcagagtgcaatccgaactgagacggcttttggagatttgcttgccctcgcgggttcgcgtcccactgtcaccgccattgtagcacgtgtgtagcccatcccgtaagggccatgaggacttgacgtcatccacaccttcctcgcggcttatcaccggcagtctccccagagtgcccaactaaatgatggcaactgaggacgtgggttgcgctcgttgcgggacttaacccaacatctcacgacacgagctgacgacagccatgcagcacctgtgtgcgcgctcccgaaggagaccgtgaatctctccacgtaacacgccatgtcaaaggatggtaaggttctgcgcgttgcttcgaattaaaccacatgctccaccgcttgtgcgggcccccgtcaattcctttgagttttaatcttgcgaccgtactccccaggcggaatgctcaaagcgttagctgcgccactgagaggcaagccccccaacggctggcattcatcgtttacggcgtggactaccagggtatctaatcctgtttgctccccacgctttcgcgcctcagcgtcagtgtcggaccagttggccgccttcgccaccggtgttcttgcgaatatctacgaatttcacctctacactcgcagttccaccaacctcttccgaactcaagtctcccagtatcgaaggcaattctgtggttgagccacaggctttcacccccgacttaaaagaccgcctacgcgccctttacgcccagtgattccgagcaacgctagcccccttcgtattaccgcggctgctggcacgaagttagccggggcttattcctccggtaccgtcattatcgtcccggataaaagagctttacaaccctaaggccttcatcactcacgcggcatggctggatcaggcttgcgcccattgtccaatattccccactgctgcctcccgtaggagtctgggccgtgtctcagtcccagtgtggctgatcatcctctcagaccagctactgatcgtcgccttggtaggccgttaccccaccaactagctaatcagacgcgggccgatcttccggcagtaaacctttccccaaaagggcgtatccggtattagctcaagtttccctgagttattccgaaccagaaggcacgttcccacgcgttactcacccgtccgccgctgacaccgaagtgcccgctcgacttgcatgtgttaagcctgccgccagcgttcgctctgagccaggatcaaactct

SEQ ID NO: 121 - DNA, 2857 bp, Methylobacterium sp.
  source 1..2857: mol_type = genomic DNA
cgctaaaacctaagcccaatagcccccccgaccaccaacgaaacgctggcgctgagcgggctaatggggaaatcaagaaaattgcataagatgtgataatctttgtcacataattgggtcttgatcctttgcgttacgggatacggcaccgtgcctacggaatttttctacaattttttcagtaagatccgcgatattgttcggatctatgctataattacagtatgctccctcaccaatttctattatatcgtctttctgtggagcctgaggcgcaggtattccgagcctcacctccttccaaaagcccagttgattaagatgtgctcgttctgagtgtgcaacacccggcttacccgcaatccaagtcacaagagtaagcccagagccaatagttgcaatataaatatcacaagcgaacgcccaggaaatactatcattcaggctcaaatttattccttcgtaaatccctaccccttcaggaacagtaatttttatttgttccgcaaattctcggcagtcaggcgtaccatccagaaaaaatgcgactttttgcccctttcttaaaatccgattagcaatatttgagactgctgttgtttgatcaatccaaaccttattgtgcgctcttagattaaaccatatcaatacatcgaatttttttgcatcttctattcttgcgttttcaatcgaactaaggacggacgttgagaatctgtgtaatttttccgtcaagcttatttctattcgaagagcagttggccgaacaagaaacatgcgttcctcaagagattttcgaaaacaatcttcggccgtatcagcgtggacgatcttgagcgatggcatttcctcaaacaaaaaggacggatcaacgaaagagtgcttataaatgagtaaattttttacggctgccagccgattctggtcttgaaaatttgaaattcccgaaagttcattccacagaaaatgtcccaagttgttcagaacgccagaaacaaccgagataacagtaggtccatttaaataatcataagataagagggagtttttgagaattagctcttgaaatttttggattgccagaattggaccgccccaatcatgaatagggttgccaagctgcaacgcaatgttcatgaaaggcaaataaataaaagatttacggcctccaaaaccagctattaccaaataaaacggctgtaaaccattaaatttaatgaataggtagctctggcgcaactcctctacgggaacaacaaaagtatgatttgaagtaagatagccccccgtaaacgggcaaacacaaaacacaagtccatatctaattgctgagtactgccagaccccatccttatctgcaagatttgaaagaaaatcatcctcaaacggtgcttcgtcagcgttatctacactaagtagatactcagcaatctctgcgtatcctaattcccgcgctagatttgcctcaaaacgaaaattatcttgggcacattctttcaccgcttgcatatttagcttaagattaaggtcgccattaaatgaacgcgtgagtacttcttcattccaagcaggtattatctgtactgcaggccgattagccgtccagtcatttattaaatccccctcaattcctgaagataacttaatgatgaggcttcgaggcacatgtttatggatcgcatcccataaattttccaaattctgaggcttcgaacttagagcttgaaaaccgtcaatccaaccattaccgtaaagactttccaatttatctcttctgataagatcaaattttacatttctgaaataaaacccagctccttcacatttaatacggacttccagagccttagcgccttcaatccagactcgaaagctaattttgtctttgaaaagaaccgaaacgattgtacgaccgctatcatacaattctaaagttccagaaaagcgggaagtctgcgcatcaatatcgagtgaaacttcgtaaaatccgtcttcaaattgacgatatggaccgtaaaatacaaatccatcaattccgtcgggaattaacagattttgactttcgtcaagatacttcttatattcttcgacaacaatcaaatctggaattctaagaactatgctttcgcccgaattgtattcagtcattctttgttccctttgttgagttaaaaggcgcttaacatttaaggccaatttcaacgaaatatagaagtcttctcgcttgttgtcatgtattaaattatatttagcaaatctaaatgtaataccatggaaatattgcgaagttgaaaaaataaaatattattacaatcgaatttttccaaaacattcctcgaaccagacacagtataccatagaaatatcataagacacggacaaattttagaatgaccagttataatcatcgcctatttttctacggaacggttattcagcgaaagaacaagaactggttgatcatttttcatctctttgtcgatattgctaagcagcattttatatgctatttgttttgatttcgttccgcacatcgattgaacacatctagctcatattatcctcggtcgagcgcttgagtatacagggtaacgcggcagattggcatcaatacgcggaccgcatagtccgggcaacaatgctttgttgagaagcgagctgatagcatgttgcgcgacggtccccactggcacggtgccatgttgaccacggtgcacataccttgtgcactaccatgttgggccgctatggcgccatcatacatttcctcaggtgggagtagacggtgtctgctctcctctctgcacctctcccagtctgacagatttttttcgccgcaatccggggatgcagcgat

SEQ ID NO: 122 - DNA, 391 bp, Methylobacterium sp.
  source 1..391: mol_type = genomic DNA
tcagtggaggtgccatggtcagtgatacgtacaatggaggacccgcctgggtctgggccgttggctggaatcagagccgggctgcaggctttgcctcccgctcaaccggatgatttggtgctcgtttgcgcggtggactctccggggattggcaggtcaattcgtcagttgcgcgaagcgctagaggatgatccgttgaaccatttcgctggggccgtcacatttgggggagtaccgaagcctcatcgtcagctcctgcaagggttgtatcggcgagtacccttagaagcctcgattgccgctgctgacacggtcgtgaaccgctcggtacacagcgtgctcggtggcttatgcttgctcgaccttccagtggagccagaggcttgtcgtg

SEQ ID NO: 123 - DNA, 843 bp, Methylobacterium sp.
  source 1..843: mol_type = genomic DNA
aagagcgcgagctgcgaccgatcacggtacgggccctcaaaagcgcgattagtatctttgacggctttaatgatgttcggaacaaccgttcctttgcgcatgacaacgaccttctcgatgcggcagaggcccggttcatcttcgagacgatcagctcaatcctccgcttcgtgaaaagcgtggatacagggcgctttggaagctgagcactggtcgaagctggctcatccaatggcaagctaatcatatgtgaaaaggttcgagcccgattagggctaagcacaccgcgtaatagcttgatagcaagacggctcgcggtgaagacgcccagatgtcttagctcactcctccggtggccgtccgccgtcgatcacggtgaagcgggagcgggctatctcgcggagccgatcacgctcttcctcttcagacagcggagctgcatccggcttgaacgggccgagcagcgcgtcggcgtgggctagaacagtggggttgcctgctccttcacccaacacctccagcaccttcacgcggaccatctcgatatcgaccccaccgcggtcccgcagaaagcccataagggccgcattgaccacgtagagagcgaggagctgatcagccaccaagttcatggccggcaccacctgaacggcgatctcctccagcctaccgatgcgagccagggggtcgctttggtcgtggtcggacataggtcacctgcacagtgctggtctctctatcttgatagcaggacggctcgacgcggtgaacgctagacgcccagatggcccgccaccttgacggatcggtaaggagacgttgagcatgatcgccggcatgacg

SEQ ID NO: 124 - DNA, 176 bp, Methylobacterium sp.
  source 1..176: mol_type = genomic DNA
tcccaaggtagcacaaaccagggcgatgcaaattccactgatgaaattttcggggtttgttcacagatagccgacacggatcggcacttatcaacacgcgcgccagttgactggtggaatatggggacagggcgcgcacaaagccttgacaccccttcgatcttcaccgctcgcca

SEQ ID NO: 125 - DNA, 226 bp, Methylobacterium sp.
  source 1..226: mol_type = genomic DNA
gcgactttttaatgctgggtcgctcggcttcttaagtgatccaccgcggattgaagaatatccgccaatcccacaagatcctctttgcgcccaggtgcgatcccaagggctactctcctctgttgattaacgtcagccgaagagaggttcaggacaaatgttactccgcccatttcgccagaaatgcgtgcggcctgatcaagcacttgcttgtcgcgggatttct

SEQ ID NO: 126 - DNA, 349 bp, Methylobacterium sp.
  source 1..349: mol_type = genomic DNA
gttcgacgtcggcctctacgacgtcacggggctgacgccgggggtgatcccgcccttcgagctcgatggcttcgagcgcgatctagcgaagtgccagcggtactatgcgactggttacgatgtattcaatcagtctctcaccgcgaatggctggggctttgaggtgcctttgccgcttcctcaagtaatgcggatcacgccctcggtatcgcagacggttagcgccaatactaacgcccaaaacatacagatttcatccggacatccacataacttgcgcattggttgtataccagttggttcgggtccgacttactttgtgtccaattggactgctaacgcgaggctt

SEQ ID NO: 127 - DNA, 1342 bp, Methylobacterium sp.
  source 1..1342: mol_type = genomic DNA
aaggaacgtgtctactgatctggcccaaatccccgccttccagacctgttcactgtcaatctgcgaagcctggatcagctggatgggtcagcctcaaacgatgagatcgacggccgcgtgaccgacgtgctcgcgctggctcccgaggtgcgtggctacgtccacaaggatggctcactaccaaacaggaacaaagccacttttgtgaaccagaagtgcagcttcctacgccagaaatcgctgcgacacgccccgtgttattatcacgttttcactcgggcaccgcaatcagcatggctccctcctctgccagcacgagcatgcggcgagcctcaagagacacaatccgtgcaaccgtattacgtccggctcccgaaccacaggaaactctgacactcacaacctgtcgtcttggaagtccaagcttctcgatgctggctcgcccgagagaccgaaattttccctgcaactcgtttgcgaccgcctcaggagtgcggcttgaaaattcataggttgcgccatcccaacatgggtccgaaaaagaggactcgaacgatctctgatcaaagcgaatacgagtcccaatcaacttctcggcaaaagagtcctttggttttggacatcccgaaatgcattgcatgtcagcaatgcgccaaggacggctgaaaaggacgcttcgctccgcctcctcacttgccgtcgcactaaagcaatagctgatcagcaatccgataagcccaacgaaggaagcactccgcatcgctaggctaccatggtcccgctgcttcgacccaacgcgactgttgccggcgagattgctctgactcgacgaaacgatttggaacggccaccccaataaggattgtcgaacgcaactttggcaacaccagtgctactttggctaccgatccactcggtgctgctgagtacgatgccaatatgattaggagccgtacctgctgcggggaagatcacgatatctccagcccgactttcggtaacatcttcaaagagattgcttgtcttgaagcctgcaacattcgtctcaaatctagatgcgttgttgggaaaaagtgctttgtacacataacatacaaatccagagcagtcgaacccatccgttgtcttacctccccacctgtaatccttgccatcggcgagttccataacttctaatgctttagaagcgatccctccaccagatacagtttgaacagagggttgcgatccagcagccatctaccgacctttcttcacttctcgcgtcacgcgatttatattttctatataaaattatacgaaaactggattgtcaattctagaaatggaactgccagccctt

SEQ ID NO: 128 - DNA, 511 bp, Methylobacterium sp.
  source 1..511: mol_type = genomic DNA
gcggacgatgagcaggaccagagcgatgatgagtgctgacgagttactgcggttcgccgcaccactcgcttaccgagtagcggataagttctttaagttcacatcatgggcaatcattgtcgtaggcctacggtacgcaaacagcgtttcgaacagtcgcgcactctcttggctcgcgtggattgccaccgacctcttttctactgccctgatcctgcaagccctctacatcgctgttagagatccagaggggctaggcgtcccaccggcagcgcggactgcttcacgcgttatccagttcgtagtcgctgttactatcggtatgttcctgacatcaccggccttatacctcgatgaggtgcttgaagcgctaaaggccgctagaccgaagtaaaggaacctgagtgctcactgtagctccgatgccgtgccgcggcgtggcatcaagcccgctaccgtcgccgtagggccggagactggaccacctacccccggcctgcttgtcccgcca

SEQ ID NO: 129 - DNA, 719 bp, Methylobacterium sp.
  source 1..719: mol_type = genomic DNA
gatatctacggacgcagcgcctaacccgtttggggttatgggtgtccaaagcttaggaatagataatggagtttgagaacaattcaaacgtgcccgtagacccggccagactaagccgggagcccagcgaaatagaaaattgctcacgtacttaatgttgtgcaggtcgttggctgcgcttcgaaccgcgccgctgtggcaagccttggtgaaattcagaaaggaagttctgcctgcggctagactgcccgataaatcgaattatgcgcaaccaaatgttttctaaaattcgtcatttagcaaaaacaagatttgagggcgaagaagggacgtgtccgactgtatatacggtttcgttcgctaatctaacttaaagccgctgaggtccgatgcgaaaagttgatacaatggcgattcgaaactctattattcggcttgcgaagctcgagattgccgccttatcgatggcgctcctgcgagatcaattaggcttcgctaataagccaggcttgctcctaagtattgaggcggatgccaaagctgccgaccaaatcgcggcatcgcctgctgcccgtaatctcccagaggacgaggggcatgcatacgtcgaggcatgcctggcagcccgtcgcatcctgatcgagacgtctgggatgtttgatgctaactggtatctattgaaaaatcccgatgtcggcgtgactggcgtcgacgctctt

SEQ ID NO: 130 - DNA, 1130 bp, Methylobacterium sp.
  source 1..1130: mol_type = genomic DNA
gtgaactagtcggccttatctggctgtgtcatcaccagggaacgcgcgctgttcttctcgaacttccatcgaccagactgctgcattcgggccacgacttcaccagcttcgatcaaacagggtaggtcagcactggctgcgccctgtttgaatgcctcacggtattccgcggctcttgcaggttctatcggttcgccgataagccagtgctgcccagagcccagtcggatgttaaaccaaatattgctaaataggaatgcgagatctttatccgatatgcactccggatctcgaatatcaattacttcaatgttctttcctgattcattcgctctttctacaatatctgtcaccttttctgtatgcgtcccatcgtgtatgatagtgagttcttgatttgtaatccgaagtattctagcatattttttccgaagatcgccttgcttccaacttactgctctcatatccctatggggtgggccaggtgatatatgaaccacccgttgtccttgatctttcaaataacgcaacaacggaatgtagtcacgatcgctggatacgagagctaaatattcgtggtctcgcatattggcatgtttaatcatctctactgtcaaaagaatatctacgccagactctgacgatctctggacggcctctccgcaattttggcacttgtgtgccgtttctttctgaacacgctcctgaaaatcaaagaacaatcctggatgatcgccgtattggtcaagaacgctatttttaaaggcgacatctctcccgttgccttgagtgtatgacccaaagatatagcatccagccaaaaccgtaccgcttccttgtccataattcggggagcattcttgaactacatgttgtgtcagttcaggccagattacatctaccgatatggctttagatgtatgacttctggcattaatcaccacattccagaaatctacgaaaacttttactctaatgttatttgattgtacgaccttaatgcgttcgcgtcccggaaaatcgaactcgccaaccatgctagtactctcttttggccggcgcagaaggcgacagtcggttgcgacatgctatattcgacagccgagtcgagcgttgcaggctcaacccga

SEQ ID NO: 131 - DNA, 177 bp, Methylobacterium sp.
  source 1..177: mol_type = genomic DNA
gaggagaagaacaaacagaacgaattgccagagaaatatcgcgacaaaatcgcagagatgctatcttggtctgacgaagattggatgatgctgcgagcgtatttcccagaagacatggagcggtttcggaagatcatcatgggcgcgtctgcttcgcagaccgggctcttgcctgac

(19) United States
(12) Patent Application Publication
(10) Pub. No.: US 2025/0024841 A1
(43) Pub. Date: Jan. 23, 2025

(54) METHYLOBACTERIUM STRAINS AND METHODS FOR ENHANCED PLANT PRODUCTION

(71) Applicant: NewLeaf Symbiotics, Inc., St. Louis, MO (US)

(72) Inventors: Natalie Breakfield, St. Louis, MO (US); Patrick Vogan, St. Louis, MO (US); Doug Bryant, Temecula, CA (US); Janne Kerovuo, Los Angeles, CA (US); Allison Jack, St. Louis, MO (US); Ashley Haddox, St. Louis, MO (US)

(73) Assignee: NewLeaf Symbiotics, Inc., St. Louis, MO (US)

(21) Appl. No.: 18/714,612

(22) PCT Filed: Dec. 1, 2022
(86) PCT No.: PCT/US2022/080735

§ 371 Date: May 30, 2024

Related U.S. Application Data
(60) Provisional application No. 63/382,626, filed on Nov. 7, 2022.
(60) Provisional application No. 63/284,878, filed on Dec. 1, 2021.

(51) Int. Cl. A01N 63/20; A01P 21/00

(52) U.S. Cl. CPC A01N 63/20; A01P 21/00

(57) ABSTRACT

Methylobacterium strains that enhance early growth of plants, improve propagation/transplant vigor, increase nutrient uptake, improve stand establishment, improve stress tolerance, and/or increase a plant's ability to utilize nutrients are provided herein.

### CROSS-REFERENCE TO RELATED APPLICATIONS

This international patent application claims the benefit of U.S. Provisional Patent Application No. 63/284,878, filed Dec. 1, 2021, and U.S. Provisional Patent Application No. 63/382,626, filed Nov. 7, 2022, the entire disclosure of which are incorporated herein by reference.

### INCORPORATION OF SEQUENCE LISTING XML

A computer readable form of the Sequence Listing XML containing the file named “NLSYM7005.WO Sequence Listing.xml,” which is 359,337 bytes in size (as measured in MICROSOFT WINDOWS® EXPLORER) and was created on Nov. 30, 2022, is provided herein and is herein incorporated by reference. This Sequence Listing consists of SEQ ID NOs: 1-131.

### BACKGROUND

Plants require certain macronutrients and micronutrients for growth and metabolism. These elements are generally found in the soil as salts and can be consumed by plants as ions. In agriculture, soil can become depleted of one or more of these nutrients requiring the addition of fertilizers to provide sufficient quantities of the nutrients for crop growth. In hydroponic systems, all nutrients must be supplied to the growing plants and are often the greatest cost for a hydroponic plant production system. Methods of enhancing plant production by improving growth and/or increasing nutrient utilization are desired.

One-carbon organic compounds such as methane and methanol are found extensively in nature and are utilized as carbon sources by bacteria classified as methanotrophs and methylotrophs. Methanotrophic bacteria include species in the genera Methylobacter, Methylomonas, Methylomicrobium, Methylococcus, Methylosinus, Methylocystis, Methylosphaera, Methylocaldum, and Methylocella (Lidstrom, 2006). Methanotrophs possess the enzyme methane monooxygenase which incorporates an atom of oxygen from O₂ into methane, forming methanol. All methanotrophs are obligate one-carbon utilizers that are unable to use compounds containing carbon-carbon bonds. Methylotrophs, on the other hand, can also utilize more complex organic compounds, such as organic acids, higher alcohols, sugars, and the like. Thus, methylotrophic bacteria are facultative methylotrophs. Methylotrophic bacteria include species in the genera Methylobacterium, Hyphomicrobium, Methylophilus, Methylobacillus, Methylophaga, Aminobacter, Methylorhabdus, Methylopila, Methylosulfonomonas, Marinosulfonomonas, Paracoccus, Xanthobacter, Ancylobacter (also known as Microcyclus), Thiobacillus, Rhodopseudomonas, Rhodobacter, Acetobacter, Bacillus, Mycobacterium, Arthobacter, and Nocardia (Lidstrom, 2006).

Some methylotrophic bacteria of the genus Methylobacterium are pink-pigmented. They are conventionally referred to as PPFM bacteria, being pink-pigmented facultative methylotrophs. Green (2005, 2006) identified twelve validated species in the genus Methylobacterium, specifically M. aminovorans, M chloromethanicum, M. dichloromethanicum, M. extorquens, M. fujisawaense, M. mesophilicum, M organophilum, M. radiotolerans, M. rhodesianum, M rhodinum, M thiocyanatum, and M. zatmanii. However, M. nodulans is a nitrogen-fixing Methylobacterium that is not a PPFM (Sy et al., 2001). Some publications have reported that other Methylobacterium species are capable of fixing nitrogen (Madhaiyan et al. (2015) Biotechnol. Biofuels: 8:222; WO2020245675) although nitrogen fixation pathway genes have not been reported to be present in those species.

### SUMMARY

Provided herein are compositions comprising one or more Methylobacterium strains that enhance early growth of plants, improve propagation/transplant vigor, increase nutrient uptake, improve stand establishment, improve stress tolerance, and/or increase a plant's ability to utilize nutrients, such as nitrogen, potassium, sulfur, cobalt, copper, zinc, phosphorus, boron, iron, and manganese, and/or that have ability fix nitrogen. In certain embodiments, the Methylobacterium in the composition comprises at least one gene encoding a 16S RNA that has at least 97%, 98%, 99%, 99.5%, or 100% sequence identity to SEQ ID NOS:91-120. In certain embodiments, the Methylobacterium in the composition is selected from the group consisting of NLS0665 (NRRL B-68194), NLS0754 (NRRL B-68197), NLS0672 (NRRL B-68196), NLS0693 (NRRL B-67926), NLS0729 (NRRL B-68195), NLS0049 (NRRL B-68236), NLS0591 (NRRL B-68215), NLS0439 (NRRL B-68216), NLS1310, NLS1312, NLS0612 (NRRL B-68237), NLS0706 (NRRL B-68238), NLS0725 (NRRL B-68239), LGP2001 (NRRL B-50930), LGP2002 (NRRL B-50931), LGP2009 (NRRL B-50938), LGP2015 (NRRL B-67340), LGP2016 (NRRL B-67341), LGP2017 (NRRL B-67741), LGP2018 (NRRL B-67742), LGP2019 (NRRL B-67743), LGP2020 (NRRL B-67892), LGP2021 (NRRL B-68032), LGP2022 (NRRL B-68033), LGP2023 (NRRL B-68034), LGP2029 (NRRL B-68065), LGP2030 (NRRL B-68066), LGP2031 (NRRL B-68067), LGP2033 (NRRL B-68068), LGP2034 (NRRL B-68069), and LGP2167 (NRRL B-67927).

In certain embodiments, the compositions provide for an increase in nitrogen use efficiency of a treated plant. In certain embodiments, the Methylobacterium in the composition is a variant of a Methylobacterium selected from the group consisting of NLS0665 (NRRL B-68194), NLS0754 (NRRL B-68197), NLS0672 (NRRL B-68196), NLS0693 (NRRL B-67926), NLS0729 (NRRL B-68195), NLS0049 (NRRL B-68236), NLS0591 (NRRL B-68215), NLS0439 (NRRL B-68216), NLS1310, NLS1312, NLS0612 (NRRL B-68237), NLS0706 (NRRL B-68238), NLS0725 (NRRL B-68239), LGP2001 (NRRL B-50930), LGP2002 (NRRL B-50931), LGP2009 (NRRL B-50938), LGP2015 (NRRL B-67340), LGP2016 (NRRL B-67341), LGP2017 (NRRL B-67741), LGP2018 (NRRL B-67742), LGP2019 (NRRL B-67743), LGP2020 (NRRL B-67892), LGP2021 (NRRL B-68032), LGP2022 (NRRL B-68033), LGP2023 (NRRL B-68034), LGP2029 (NRRL B-68065), LGP2030 (NRRL B-68066), LGP2031 (NRRL B-68067), LGP2033 (NRRL B-68068), LGP2034 (NRRL B-68069), and LGP2167 (NRRL B-67927). Plants, plant parts and seeds coated or partially coated with such compositions are also provided herein. In certain embodiments, the plants are leafy green plants, including microgreens and/or herbs. In certain embodiments, the plants are fruit or vegetable plants. In certain embodiments, the plants are agricultural row crops. In certain embodiments, the plants are grown in a greenhouse. In certain embodiments, the plants are grown hydroponically or aeroponically.

Also provided are isolated Methylobacterium selected from NLS0665 (NRRL B-68194), NLS0754 (NRRL B-68197), NLS0672 (NRRL B-68196), NLS0729 (NRRL B-68195), NLS0049 (NRRL B-68236), NLS0591 (NRRL B-68215), NLS0439 (NRRL B-68216), NLS1310, NLS1312, NLS0612 (NRRL B-68237), NLSO706 (NRRL B-68238), and NLS0725 (NRRL B-68239); and compositions comprising such Methylobacterium isolates or variants thereof. In certain embodiments, the isolated Methylobacterium or the Methylobacterium in the compositions or variants thereof comprise at least one gene encoding a 16S RNA of SEQ ID NOS: 108-119. Also provided are compositions comprising a fermentation product comprising a Methylobacterium strain that is essentially free of contaminating microorganisms. In certain embodiments, the Methylobacterium strain is selected from the group consisting of NLS0665 (NRRL B-68194), NLS0754 (NRRL B-68197), NLS0672 (NRRL B-68196), NLS0693 (NRRL B-67926), NLS0729 (NRRL B-68195), NLS0049 (NRRL B-68236), NLS0591 (NRRL B-68215), NLS0439 (NRRL B-68216), NLS1310, NLS1312, NLS0612 (NRRL B-68237), NLS0706 (NRRL B-68238), NLS0725 (NRRL B-68239), LGP2001 (NRRL B-50930), LGP2002 (NRRL B-50931), LGP2009 (NRRL B-50938), LGP2015 (NRRL B-67340), LGP2016 (NRRL B-67341), LGP2017 (NRRL B-67741), LGP2018 (NRRL B-67742), LGP2019 (NRRL B-67743), LGP2020 (NRRL B-67892), LGP2021 (NRRL B-68032), LGP2022 (NRRL B-68033), LGP2023 (NRRL B-68034), LGP2029 (NRRL B-68065), LGP2030 (NRRL B-68066), LGP2031 (NRRL B-68067), LGP2033 (NRRL B-68068), LGP2034 (NRRL B-68069), LGP2167 (NRRL B-67927), and variants thereof. In certain embodiments, a variant of a Methylobacterium strain in the compositions herein comprises a gene encoding a 16S RNA that has at least 97%, 98%, 99%, 99.5%, or 100% sequence identity to SEQ ID NOS: 91-120, and or a marker sequence of SEQ ID NOS: 1-3, SEQ ID NOS: 13-15, SEQ ID NOS: 25-27. SEQ ID NOS: 37-39, SEQ ID NOS:49-51, SEQ ID NOS: 61-64, SEQ ID NOS: 71-73, SEQ ID NOS: 74-76 or SEQ ID NOS: 121-131. In certain embodiments, the composition further comprises an an additional active ingredient, an agriculturally acceptable adjuvant, and/or an agriculturally acceptable excipient.

Additionally, isolated Methylobacterium selected from NLS0665 (NRRL B-68194), NLS0754 (NRRL B-68197), NLS0672 (NRRL B-68196), NLS0729 (NRRL B-68195), NLS0049 (NRRL B-68236), NLS0591 (NRRL B-68215), or NLS0439 (NRRL B-68216); and compositions comprising such Methylobacterium isolates or variants thereof are disclosed.

In certain embodiments, the Methylobacterium isolates in the compositions provided herein comprise one or more genetic elements associated with the ability to enhance early plant growth, wherein the one or more genetic elements (i) is recD2_2 or pinR; or (ii) the one or more genetic elements encode a protein having a consensus amino acid sequence of SEQ ID NO: 77 to SEQ ID NO: 83. In some embodiments, Methylobacterium isolates in the compositions provided herein that improve early plant growth also impart one or more additional beneficial traits to treated plants or plants grown from treated plant parts or seeds, wherein the trait is enhanced uptake of nutrients, enhanced assimilation of nutrients, and/or enhanced nutrient use efficiency. In some embodiments, plants treated with Methylobacterium isolates provided herein demonstrate enhanced nitrogen use efficiency.

Methods of improving the production of plants by applying one or more Methylobacteirum strains to the plant, a plant part, or a seed are provided herein. In some embodiments, the composition comprising one or more Methylobacterium strains is applied such that it coats or partially coats the plant, plant part, or seed. In some embodiments, plant production is improved by enhancing early plant growth. In some embodiments, plant production is improved by increasing rooting of the plant. In some embodiments, plant production is improved by enhancing propagation/transplant vigor. In some embodiments, plant production is improved by enhancing stand establishment. In some embodiments, plant production is improved by enhancing stress tolerance. In some embodiments, plant production is improved by increasing the content of nutrients present in the plant or a plant part. In certain embodiments, the content of one or more nutrients selected from the group consisting of nitrogen, potassium, sulfur, copper, zinc, phosphorus, boron, iron, and manganese is increased. In certain embodiments, the nitrogen content in the plant is increased. In certain embodiments of such methods, the Methylobacterium in the composition is selected from the group consisting of NLS0665 (NRRL B-68194), NLS0754 (NRRL B-68197), NLS0672 (NRRL B-68196), NLS0693 (NRRL B-67926), NLS0729 (NRRL B-68195), NLS0049 (NRRL B-68236), NLS0591 (NRRL B-68215), NLS0439 (NRRL B-68216), NLS1310, NLS1312, NLS0612 (NRRL B-68237), NLSO706 (NRRL B-68238), NLS0725 (NRRL B-68239), LGP2001 (NRRL B-50930), LGP2002 (NRRL B-50931), LGP2009 (NRRL B-50938), LGP2015 (NRRL B-67340), LGP2016 (NRRL B-67341), LGP2017 (NRRL B-67741), LGP2018 (NRRL B-67742), LGP2019 (NRRL B-67743), LGP2020 (NRRL B-67892), LGP2021 (NRRL B-68032), LGP2022 (NRRL B-68033), LGP2023 (NRRL B-68034), LGP2029 (NRRL B-68065), LGP2030 (NRRL B-68066), LGP2031 (NRRL B-68067), LGP2033 (NRRL B-68068), LGP2034 (NRRL B-68069), and LGP2167 (NRRL B-67927). For example, in various embodiments, methods for enhancing plant production comprise: (a) applying a composition to a plant, plant part, or seed, wherein the composition comprises at least one Methylobacterium selected from the group consisting of NLS0665 (NRRL B-68194), NLS0754 (NRRL B-68197), NLS0672 (NRRL B-68196), NLS0693 (NRRL B-67926), NLS0729 (NRRL B-68195), NLS0049 (NRRL B-68236), NLS0591 (NRRL B-68215), NLS0439 (NRRL B-68216), NLS1310, NLS1312, NLS0612 (NRRL B-68237), NLS0706 (NRRL B-68238), NLS0725 (NRRL B-68239), LGP2021 (NRRL B-68032), LGP2022 (NRRL B-68033), LGP2023 (NRRL B-68034), LGP2029 (NRRL B-68065), LGP2030 (NRRL B-68066), LGP2031 (NRRL B-68067), LGP2033 (NRRL B-68068), LGP2034 (NRRL B-68069), and variants thereof, and, (b) growing the plant to at least a two leaf stage, thereby enhancing at least one plant trait selected from the group consisting of early plant growth, propagation/transplant vigor, nutrient uptake, stand establishment, stress tolerance and nutrient utilization efficiency; wherein said trait is enhanced in comparison to an untreated control plant that had not received an application of the composition or in comparison to a control plant grown from an untreated seed that had not received an application of the composition. In some embodiments, the Methylobacterium in the composition is selected from LGP2001 (NRRL B-50930), LGP2002 (NRRL B-50931), LGP2009 (NRRL B-50938), LGP2015 (NRRL B-67340), LGP2022 (NRRL B-68033), a combination of LGP2002 (NRRL B-50931) and LGP2015 (NRRL B-67340), and other combinations and variants thereof. In certain embodiments, the composition is applied such that it coats or partially coats the plant, plant part, or seed. In certain embodiments, the plant is selected from the group consisting of rosemary, French tarragon, basil, oregano, Pennisetum, and/or other herbs. In certain embodiments, the Methylobacterium in the composition is a variant of any of the aforementioned Methylobacterium isolates. In certain embodiments, the plants are leafy green plants. In certain embodiments, the leafy green plant is selected from the group consisting of spinach, lettuce, beets, swiss chard, watercress, kale, collards, escarole, arugula, endive, bok choy, and turnips. In certain embodiments, plant biomass is increased by treatment with one or more Methylobacterium strains as provided herein. In some embodiments, enhanced early growth is assessed at the two true leaf stage of development. In certain embodiments of the methods provided herein, the Methylobacterium compositions are applied to plants, plant parts, or seeds of fruits or vegetables grown hydroponically. In some embodiments, the Methylobacterium compositions provided herein are applied to plants, plant parts, or seeds of leafy green vegetables. In some embodiments, such leafy green vegetables are grown hydroponically. In certain embodiments, the plants are agricultural row crops. In certain embodiments, the plants are rice plants.

In certain embodiments of methods to improve plant production provided herein, the plant is a leafy green plant, the plant improvement comprises enhanced early growth, improved propagation/transplant vigor, improved stand establishment, improved stress tolerance, and/or increased levels of nutrients in the plant or plant part and the Methylobacterium is selected from NLS0665 (NRRL B-68194), NLS0754 (NRRL B-68197), NLS0672 (NRRL B-68196), NLS0693 (NRRL B-67926), NLS0729 (NRRL B-68195), NLS0049 (NRRL B-68236), NLS0591 (NRRL B-68215), NLS0439 (NRRL B-68216), NLS1310, NLS0612 (NRRL B-68237), NLS0706 (NRRL B-68238), NLS0725 (NRRL B-68239), LGP2001 (NRRL B-50930), LGP2002 (NRRL B-50931), LGP2009 (NRRL B-50938), LGP2015 (NRRL B-67340), LGP2016 (NRRL B-67341), LGP2017 (NRRL B-67741), LGP2018 (NRRL B-67742), LGP2019 (NRRL B-67743), LGP2020 (NRRL B-67892), LGP2021 (NRRL B-68032), LGP2022 (NRRL B-68033), LGP2023 (NRRL B-68034), LGP2029 (NRRL B-68065), LGP2030 (NRRL B-68066), LGP2031 (NRRL B-68067), LGP2033 (NRRL B-68068), LGP2034 (NRRL B-68069), LGP2167 (NRRL B-67927), and variants thereof. In some embodiments, the leafy green plant is selected from the group consisting of spinach, lettuce, beets, swiss chard, watercress, kale, collards, escarole, arugula, endive, bok choy, and turnips. In some embodiments, the Methylobacterium is selected from LGP2001 (NRRL B-50930), LGP2002 (NRRL B-50931), LGP2009 (NRRL B-50938), LGP2015 (NRRL B-67340), LGP2022 (NRRL B-68033), a combination of LGP2002 (NRRL B-50931) and LGP2015 (NRRL B-67340), and other combinations and variants thereof. In some embodiments, the leafy green plant comprises rosemary, French tarragon, basil, oregano, Pennisetum, and/or other herbs. In certain embodiments of methods to improve plant production provided herein, the plant is a cannabis plant, the plant improvement is selected from enhanced growth and/or rooting, decreased cycling time, and increased biomass or yield, and the Methylobacterium is selected from LPG2001 (NRRL B-50930), LGP2002 (NRRL B-50931), LGP2009 (NRRL B-50938), LGP2019 (NRRL B-67743), and variants thereof. In certain embodiments of methods to improve plant production provided herein, a variant of LGP2002 has genomic DNA comprising one or more polynucleotide marker fragments of at least 50, 60, 100, 120, 180, 200, 240, or 300 nucleotides of SEQ ID NOS: 13-15. In certain embodiments, a variant of LGP2009 has genomic DNA comprising one or more polynucleotide marker fragments of at least 50, 60, 100, 120, 180, 200, 240, or 300 nucleotides of SEQ ID NOS: 71-73. In certain embodiments, a variant of LGP2019 (NRRL B-67743) has genomic DNA comprising one or more polynucleotide marker fragments of at least 50, 60, 100, 120, 180, 200, 240, or 300 nucleotides of SEQ ID NOS: 25-27.

In certain embodiments, methods of enhancing growth and/or yield of a plant by treatment with a Methylobacterium isolate disclosed herein are provided. In some embodiments of such methods, the Methylobacterium is selected from NLS0665 (NRRL B-68194), NLS0754 (NRRL B-68197), NLS0672 (NRRL B-68196), NLS0693 (NRRL B-67926), NLS0729 (NRRL B-68195), NLS0049 (NRRL B-68236), NLS0591 (NRRL B-68215), NLS0439 (NRRL B-68216), NLS1310, NLS1312, NLS0612 (NRRL B-68237), NLS0706 (NRRL B-68238), NLS0725 (NRRL B-68239), LGP2001 (NRRL B-50930), LGP2002 (NRRL B-50931), LGP2009 (NRRL B-50938), L (NRRL B-68238)GP2015 (NRRL B-67340), LGP2016 (NRRL B-67341), LGP2017 (NRRL B-67741), LGP2018 (NRRL B-67742), LGP2019 (NRRL B-67743), LGP2020 (NRRL B-67892), LGP2021 (NRRL B-68032), LGP2022 (NRRL B-68033), LGP2023 (NRRL B-68034), LGP2029 (NRRL B-68065), LGP2030 (NRRL B-68066), LGP2031 (NRRL B-68067), LGP2033 (NRRL B-68068), LGP2034 (NRRL B-68069), LGP2167 (NRRL B-67927), and variants thereof, and uptake and/or utilization of one or more nutrient components of a fertilizer applied during growth of said plant is enhanced. In some embodiments the one or more nutrient components is selected from the group consisting of nitrogen, phosphorus, potassium, and iron. In some embodiments, the plant is an agricultural row crop. In some embodiments, the plant is a leafy green plant, and in some embodiments the leafy green plant is grown in a hydroponic or aeroponic plant growth system. In some embodiments, a Methylobacterium treated plant can be cultivated using reduced rates of fertilizer as compared to standard application rates for said plant. In some embodiments, fertilizer application can be reduced by 5%, 10%, 15%, 20%, 25%, 30%, 35%, 40% or more. In certain embodiments, application of fertilizer can be reduced by at least 25%. In some embodiments the amount of one or more components of said fertilizer is reduced. In some embodiments levels of nitrogen, phosphorus, potassium and/or iron are reduced by 5%, 10%, 15%, 20%, 25%, 30%, 35%, 40% or more. Also provided are food products with enhanced content of nutrients as the result of treatment with Methylobacterium isolates and compositions provided herein. In some embodiments, the content of one or more nutrients selected from the group consisting of nitrogen, potassium, sulfur, copper, zinc, phosphorus, boron, iron, and manganese is increased.

Also provided herein are methods of improving growth and yield of rice plants by treating rice plants, plant parts, or seeds with one or more Methylobacterium isolates. In some embodiments, harvested seed yield and/or nutrient content of rice plants is improved. In some embodiments, rice seeds are treated and such treatment provides for increased rice seed yield. In some embodiments, the Methylobacterium isolate is selected from the group consisting of LGP2016 (NRRL B-67341), LGP2017 (NRRL B-67741), LGP2019 (NRRL B-67743), LGP2020 (NRRL B-67892), NLS0754 and NLS0665 (NRRL B-68194), and variants of these isolates. In certain embodiments bushels per acre yield of rice plants is increased by at least 2-10%. In some embodiments, rice yield is increased by 2, 3, 4, 5, 6, 7, 8, 9, 10, 12, or 15% or more. Rice plants, plant parts, or seeds coated with Methylobacterium isolates and/or compositions are also provided herein. In certain embodiments, the Methylobacterium has chromosomal genomic DNA having at least 99%, 99.9, 99.8, 99.7, 99.6%, or 99.5% sequence identity to chromosomal genomic DNA of LGP2016 (NRRL B-67341), LGP2017 (NRRL B-67741), LGP2019 (NRRL B-67743), LGP2020 (NRRL B-67892)), NLS0754 or NLS0665 (NRRL B-68194). In certain embodiments, the Methylobacterium has genomic DNA comprising one or more polynucleotide marker fragments of at least 50, 60, 100, 120, 180, 200, 240, or 300 nucleotides of SEQ ID NOS: 37-39, SEQ ID NOS: 25-27, or SEQ ID NOS: 74-76.

Also provided herein are methods of improving growth and production of cannabis plants by treating cannabis plants, plant parts, or seeds with one or more Methylobacterium isolates. In some embodiments, nutrient content of treated plants is improved. In some embodiments, a cannabis cutting from a mature plant is treated. In some embodiments, a cannabis cutting is treated by immersion in a Methylobacterium suspension. In some embodiments, the Methylobacterium is present in said suspension at a concentration of greater than 1×10³ colony forming units (CFU) per milliliter. In some embodiments, such treatments improve plant growth and rooting of such cuttings. In some embodiments, such treatments provided for a decreased cycling time for production of a cannabis plant as the result of such increased plant growth and rooting. In some embodiments, the Methylobacterium isolate is selected from the group consisting of LPG2001 (NRRL B-50930), LGP2002 (NRRL B-50931), LGP2009 (NRRL B-50938), LGP2019 (NRRL B-67743), and variants of these isolates. For example, in various embodiments, methods for enhancing plant growth and/or rooting of a cannabis plant comprise: (a) treating a cannabis plant, plant part, or seed with a composition comprising at least one Methylobacterium isolate; and (b) growing the treated plant or growing a plant from the treated plant part or seed to allow production of a rooted plant, wherein plant growth and/or rooting of the cannabis plant is increased in comparison to an untreated control plant that had not received treatment with the composition or in comparison to a control plant grown from an untreated plant part or seed that had not received treatment with the composition. Cannabis plants, plant parts, or seeds coated with Methylobacterium isolates and/or compositions are also provided herein. Various embodiments include a cannabis plant, part or seed that is at least partially coated with a composition comprising a Methylobacterium isolate selected from the group consisting of LPG2001 (NRRL B-50930), LGP2002 (NRRL B-50931), LGP2009 (NRRL B-50938), LGP2019 (NRRL B-67743), and a variant of LPG2001 (NRRL B-50930), LGP2002 (NRRL B-50931), LGP2009 (NRRL B-50938), or LGP2019 (NRRL B-67743), wherein said cannabis plant or a cannabis plant grown from said cannabis plant part or seed demonstrates enhanced plant growth or rooting, or decreased cycling time from cutting to mature plant, in comparison to a control cannabis plant that was not treated with said Methylobacterium or a cannabis plant grown from a control cannabis plant part or seed that was not treated with said Methylobacterium. In certain embodiments, the Methylobacterium has chromosomal genomic DNA having at least 99%, 99.9%, 99.8%, 99.7%, 99.6%, or 99.5% sequence identity to chromosomal genomic DNA of LPG2001 (NRRL B-50930), LGP2002 (NRRL B-50931), LGP2009 (NRRL B-50938), or LGP2019 (NRRL B-67743). In certain embodiments, the Methylobacterium has genomic DNA comprising one or more polynucleotide marker fragments of at least 50, 60, 100, 120, 180, 200, 240, or 300 nucleotides of SEQ ID NOS: 13-15, SEQ ID NOS: 71-73, or SEQ ID NOS: 25-27.

Also provided herein are methods of increasing cannabidiol (CBD) content in a cannabis plant, plant part, or seed. In various embodiments, the methods comprise: (a) treating a cannabis plant, plant part, or seed with a composition comprising at least one Methylobacterium isolate; and (b) growing the treated plant or growing a plant from the treated plant part or seed to allow production of a rooted plant, wherein CBD content of the cannabis plant is increased in comparison to an untreated control plant that had not received treatment with the composition or in comparison to a control plant grown from an untreated plant part or seed that had not received treatment with the composition. In some embodiments, CBD content can be increased by 5%, 10%, 15%, 20%, 25%, 30%, 35%, 40%, or more.

In certain embodiments of the compositions and methods provided herein, the composition further comprises at least one additional component selected from the group consisting of an additional active ingredient, an agriculturally acceptable adjuvant, and an agriculturally acceptable excipient. An additional active ingredient can be, for example, a pesticide or a second biological. In certain embodiments, the pesticide can be an insecticide, a fungicide, an herbicide, a nematicide, or other biocide. The second biological could be a strain that improves yield or controls an insect, pest, fungi, weed, or nematode. In some embodiments, a second biological is a second Methylobacterium strain. In certain embodiments of the compositions and methods provided herein, one or more additional Methylobacterium strains disclosed in Table 1 herein may be employed.

In certain embodiments of any of the aforementioned methods, the composition comprises the Methylobacterium at a titer of greater than 1×10³ CFU/gm or at a titer of about 1×10⁶ CFU/gm to about 1×10¹⁴ CFU/gm for a solid composition or at a titer of greater than 1×10³ CFU/ml or at a titer of about 1×10⁶ CFU/mL to about 1×10¹¹ CFU/mL for a liquid composition.

Various methods for selecting a Methylobacterium isolate capable of improving early plant growth are also provided. In some embodiments, the method comprises: a) detecting in the genome of a Methylobacterium isolate, one or more genetic elements, wherein said genetic element i) encodes a recD2_2 or pinR protein; or ii) encodes a protein having a consensus amino acid sequence selected from the group consisting of SEQ ID NO: 77 to SEQ ID NO: 83; and b) treating a plant, plant part, or seed with said Methylobacterium isolate, and measuring early growth of said plant to identify improved early growth in comparison to a control plant not treated with said Methylobacterium isolate. In certain embodiments, the genetic element encodes a protein having at least 50%, 60%, 70%, 75%, 80%, 85%, 90%, or 95% sequence identity to a protein having an amino acid sequence selected from the group consisting of SEQ ID NO: 84 to SEQ ID NO: 90. In certain embodiments, the genetic element encodes a protein having at least 50% sequence identity to a protein having an amino acid sequence selected from the group consisting of SEQ ID NO: 84 to SEQ ID NO: 90. In certain embodiments, the genetic element encodes a protein has an amino acid sequence selected from the group consisting of SEQ ID NO: 84 to SEQ ID NO: 90. In certain embodiments, the plant is a rice lettuce, or spinach plant.

Also provided herein is a method for enhancing plant production that comprises (a) applying a composition to a plant, plant part, or seed, wherein the composition comprises at least one Methylobacterium selected from the group consisting of LPG2001 (NRRL B-50930), LGP2002 (NRRL B-50931), LGP2009 (NRRL B-50938), LGP2015 (NRRL B-67340), LGP2022 (NRRL B-68033), a combination of LGP2002 (NRRL B-50931) and LGP2015 (NRRL B-67340), and other combinations and variants thereof, and, (b) growing the plant, thereby enhancing at least one plant trait selected from the group consisting of early plant growth, propagation/transplant vigor, nutrient uptake, stand establishment, stress tolerance, and nutrient utilization efficiency; wherein said trait is enhanced in comparison to an untreated control plant that had not received an application of the composition or in comparison to a control plant grown from an untreated seed that had not received an application of the composition; and wherein the plant is selected from the group consisting of microgreens and herbs. In certain embodiments, the herb is selected from the group consisting of rosemary, French tarragon, basil, oregano and Pennisetum.

### DETAILED DESCRIPTION

### Definitions

The term “and/or” where used herein is to be taken as specific disclosure of each of the two or more specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the terms “include,” “includes,” and “including” are to be construed as at least having the features or encompassing the items to which they refer while not excluding any additional unspecified features or unspecified items.

As used herein, the term “biological” refers to a component of a composition for treatment of plants or plant parts comprised of or derived from a microorganism. Biologicals include biocontrol agents, other beneficial microorganisms, microbial extracts, natural products, plant growth activators or plant defense agents. Non-limiting examples of biocontrol agents include bacteria, fungi, beneficial nematodes, and viruses. In certain compositions, a biological can comprise a mono-culture or co-culture of Methylobacterium, or a combination of Methylobacterium strains or isolates that have been separately cultured.

As used herein, a “leafy green plant” refers to a vegetable crop with edible leaves and includes, without limitation, spinach, kale, lettuce (including but not limited to romaine, butterhead, iceberg, and loose leaf lettuces), collard greens, cabbage, beet greens, watercress, swiss chard, arugula, escarole, endive, bok choy, and turnip greens. Leafy green plants as used herein also refers to plants grown for harvest of microgreens and/or herbs, including but not limited to lettuce, cauliflower, broccoli, cabbage, watercress, arugula, garlic, onion, leek, amaranth, swill chard, been, spinach, melon, cucumber, squash, basil, celery, cilantro, radish, radicchio, chicory, dill, rosemary, French tarragon, basil, Pennisetum, carrot, fennel, beans, peas, chickpeas, and lentils. Leafy green plants also refer to mixes of assorted leafy green plants, such as mesclun or other mixed salad greens or mixed microgreens. “Leafy green plants” as used herein also encompasses other brassica or cruciferous field greens not specifically mentioned herein by name.

As used herein, a “fruit” or “fruit bearing plant” is a fleshy fruit bearing plant, including but not limited to, melon (including watermelon and cantaloupe), berry (including strawberry, blueberry, blackberry, and raspberry), grape, kiwi, mango, papaya, pineapple, banana, pepper, tomato, squash, and cucumber plants.

As used herein, the term “Methylobacterium” refers to genera and species in the methylobacteriaceae family, including bacterial species in the Methylobacterium genus and proposed Methylorubrum genus (Green and Ardley (2018)). Methylobacterium includes pink-pigmented facultative methylotrophic bacteria (PPFM) and also encompasses the non-pink-pigmented Methylobacterium nodulans, as well as colorless mutants of Methylobacterium isolates. For example, and not by way of limitation, “Methylobacterium” refers to bacteria of the species listed below as well as any new Methylobacterium species that have not yet been reported or described that can be characterized as Methylobacterium or Methylorubrum based on phylogenetic analysis: Methylobacterium adhaesivum; Methylobacterium oryzae; Methylobacterium aerolatum; Methylobacterium oxalidis; Methylobacterium aquaticum; Methylobacterium persicinum; Methylobacterium brachiatum; Methylobacterium phyllosphaerae; Methylobacterium brachythecii; Methylobacterium phyllostachyos; Methylobacterium bullatum; Methylobacterium platani; Methylobacterium cerastii; Methylobacterium pseudosasicola; Methylobacterium currus; Methylobacterium radiotolerans; Methylobacterium dankookense; Methylobacterium soli; Methylobacterium frigidaeris; Methylobacterium specialis; Methylobacterium fujisawaense; Methylobacterium tardum; Methylobacterium gnaphalii; Methylobacterium tarhaniae; Methylobacterium goesingense; Methylobacterium thuringiense; Methylobacterium gossipiicola; Methylobacterium trifolii; Methylobacterium gregans; Methylobacterium variabile; Methylobacterium haplocladii; Methylobacterium aminovorans (Methylorubrum aminovorans); Methylobacterium hispanicum; Methylobacterium extorquens (Methylorubrum extorquens); Methylobacterium indicum; Methylobacterium podarium (Methylorubrum podarium); Methylobacterium iners; Methylobacterium populi (Methylorubrum populi); Methylobacterium isbiliense; Methylobacterium pseudosasae (Methylorubrum pseudosasae); Methylobacterium jeotgali; Methylobacterium rhodesianum (Methylorubrum rhodesianum); Methylobacterium komagatae; Methylobacterium rhodinum (Methylorubrum rhodinum); Methylobacterium longum; Methylobacterium salsuginis (Methylorubrum salsuginis); Methylobacterium marchantiae; Methylobacterium suomiense (Methylorubrum suomiense; Methylobacterium mesophilicum; Methylobacterium thiocyanatum (Methylorubrum thiocyanatum); Methylobacterium nodulans; Methylobacterium zatmanii (Methylorubrum zatmanii); or Methylobacterium organophilum.

“Colonization efficiency” as used herein refers to the relative ability of a given microbial strain to colonize a plant host cell or tissue as compared to non-colonizing control samples or other microbial strains. Colonization efficiency can be assessed, for example and without limitation, by determining colonization density, reported for example as colony forming units (CFU) per mg of plant tissue, or by quantification of nucleic acids specific for a strain in a colonization screen, for example using qPCR.

As used herein “mineral nutrients” (also sometime refered to simply as “nutrients”) are micronutrients or macronutrients required or useful for plants or plant parts including for example, but not limited to, nitrogen (N), potassium (K), calcium (Ca), magnesium (Mg), phosphorus (P), and sulfur (S), and the micronutrients chlorine (Cl), Iron (Fe), Boron (B), manganese (Mn), zinc (Z), cobalt (Co), copper (Cu), molybdenum (Mo), and nickel (Ni).

As used herein, “vitamins” are organic compounds required in small amounts for normal growth and metabolism. Vitamins are important for human and/or animal growth, and some vitamins have been reported to be beneficial to plants. Vitamins include but are not limited to vitamin A (including but not limited to all-trans-retinol and all-trans-retinyl-esters, as well as all-trans-beta-carotene and other provitamin A carotenoids), vitamin B1 (thiamine), vitamin B2 (riboflavin), vitamin B3 (niacin), vitamin B5 (pantothenic acid), vitamin B6 (pyridoxine), vitamin B7 (biotin), vitamin B9 (folic acid or folate), vitamin B12 (cobalamins), vitamin C (ascorbic acid), vitamin D (calciferols), vitamin E (tocopherols and tocotrienols), and vitamin K (quinones).

As used herein “fertilizer” can be a single nutrient nitrogen fertilizer, such as urea, ammonia, or ammonia solutions (including ammonium nitrate, ammonium sulfate, calcium ammonium nitrate, and urea ammonium nitrate). In certain embodiments, the fertilizer can be a single nutrient phosphate fertilizer, such as a superphosphate or triple superphosphate or mixtures thereof, including double superphosphate. In certain embodiments, the fertilizer can be a single nutrient potassium-based fertilizer, such as muriate of potash. In certain embodiments, the compositions comprise multinutrient fertilizers including binary fertilizers (NP, NK, PK), including, for example monoammonium phosphate, diammonium phosphate, potassium nitrate, and potassium chloride. In further embodiments, three-component fertilizers (NPK) providing nitrogen, phosphorus, and potassium are present in the aqueous compositions. In still further embodiments, the fertilizer comprises micronutrients, which may be chelated or non-chelated. In some embodiments, combinations of various fertilizers can be present in the aqueous solution, including combinations of nitrogen, phosphorus, and/or micronutrient fertilizers. Nutrient solutions provided in hydroponic plant growth systems are also considered “fertilizers” in methods and compositions described herein.

As used herein, the term “strain” shall include all isolates of such strain.

As used herein, “variant” when used in the context of a Methylobacterium isolate, refers to any isolate that has chromosomal genomic DNA with at least 99%, 99.9%, 99.8%, 99.7%, 99.6%, or 99.5% sequence identity to chromosomal genomic DNA of a reference Methylobacterium isolate, such as, for example, a deposited Methylobacterium isolate provided herein. A variant of an isolate can be obtained from various sources including soil, plants or plant material, and water, particularly water associated with plants and/or agriculture. Variants include derivatives obtained from deposited isolates. Methylobacterium isolates or strains can be sequenced (for example as taught by Sanger et al. (1977), Bentley et al. (2008) or Caporaso et al. (2012)) and genome-scale comparison of the sequences conducted (Konstantinidis et al. (2005)) using sequence analysis tools, such as BLAST, as taught by Altschul et al. (1990) or clustalw (www.ebi.ac.uk/Tools/msa/clustalw2/). Variants can be identfied, for example, by the presence of a 16S sequence of a reference strain, where the variant also demonstrates a plant production enhancement trait of the reference strain. Variants of Methylobacterium LGP2002 (NRRL B-50931), LGP2001 (NRRL B-50930), LGP2015 (NRRL B-67340), LGP2021 (NRRL B-68032), LGP2020 (NRRL B-67892), LGP2017 (NRRL B-67741), LGP2018 (NRRL B-67742), LGP2029 (NRRL B-68065), LGP2030 (NRRL B-68066), LGP2019 (NRRL B-67743), LGP2031 (NRRL B-68067), LGP2016 (NRRL B-67341), LGP2033 (NRRL B-68068), LGP2034 (NRRL B-68069), LGP2022 (NRRL B-68033), LGP2023 (NRRL B-68034), LGP2167 (NRRL B-67927), NLS1310, NLS0612 (NRRL B-68237), NLS1312NLS0706 (NRRL B-68238), NLS0725 (NRRL B-68239), NLS0665 (NRRL B-68194), NLS0729 (NRRL B-68195), NLS0672 (NRRL B-68196), NLS0754 (NRRL B-68197), NLS0591 (NRRL B-68215), NLS0439 (NRRL B-68216), NLS0049 (NRRL B-68236), and NLS0693 (NRRL B-67926), include, for example, Methylobacterium that comprise at least one gene encoding a 16S RNA that has at least 97%, 98%, 99%, 99.5%, or 100% sequence identity to SEQ ID NOS: 91-120, respectively, or comprises a marker sequence with at least 97%, 98%, 99%, 99.5%, or 100% sequence identity to SEQ ID NOS: 121-131.

As used herein, “derivative” when used in the context of a Methylobacterium isolate, refers to any Methylobacterium that is obtained from a deposited Methylobacterium isolate provided herein. Derivatives of a Methylobacterium isolate include, but are not limited to, derivatives obtained by selection, derivatives selected by mutagenesis and selection, and genetically transformed Methylobacterium obtained from a Methylobacterium isolate. A “derivative” can be identified, for example, based on genetic identity to the strain or isolate from which it was obtained and will generally exhibit chromosomal genomic DNA with at least 99%, 99.9%, 99.8%, 99.7%, 99.6%, or 99.5% sequence identity to chromosomal genomic DNA of the strain or isolate from which it was derived.

As used herein, “sequence identity” when used to evaluate whether a particular Methylobacterium strain is a variant or derivative of a Methylobacterium strain provided herein refers to a measure of nucleotide-level genomic similarity between the coding regions of two genomes. Sequence identity between the coding regions of bacterial genomes can be calculated, for example, by determining the Average Nucleotide Identity (ANI) score using FastANI (Jain et al. “High throughput ANI analysis of 90K prokaryotic genomes reveals clear species boundaries”, Nat Communications 9, 5114 (2018)) and Han et al. (“ANI tools web: a web tool for fast genome comparison within multiple bacterial strains”; Database, 2016, 1-5).

As used herein, a “correlation” is a statistical measure that indicates the extent to which two or more variables, here plant growth enhancement and identified genetic elements, occur together. A positive correlation indicates that a microbial strain containing a given genetic element is likely to enhance plant growth.

As used herein, a “pan-genome” is the entire set of genes for the microbial population being screened in a plant colonization efficiency screen. Thus, a pan-genome may represent the entire set of genes for a particular species, or the entire set of genes in multiple different species of the same genus or even the entire set of genes for multiple species classified in more than a single genus, where the strains in the population are from closely related genera.

As used herein a “genetic element” refers to an element in a DNA or RNA molecule that comprises a series of adjacent nucleotides at least 20 nucleotides in length and up to 50, 100, 1000, or 10000 or more nucleic acids in length. A genetic element may comprise different groups of adjacent nucleic acids, for example, where the genome of a plant-associated microorganism contains introns and exons. The genetic element may be present on a chromosome or on an extrachromosomal element, such as a plasmid. In eukaryotic plant-associated microorganisms, the genetic element may be present in the nucleus or in the mitochondria. In some embodiments, the genetic element is a functional genetic element (e.g., a gene) that encodes a protein.

As used herein, the terms “homologous”’ or “homologue” or “ortholog” refer to related genetic elements or proteins encoded by the genetic elements that are determined based on the degree of sequence identity. These terms describe the relationship between a genetic element or encoded protein found in one isolate, species, or strain and the corresponding or equivalent genetic element or protein in another isolate, species, or strain. As used herein, a particular genetic element in a first isolate, species, or strain is considered equivalent to a genetic element present in a second isolate, species, or strain when the proteins encoded by the genetic element in the isolates, species, or strains have at least 50 percent identity. Percent identity can be determined using a number of software programs available in the art including BLASTP, ClustalW, ALLALIGN, DNASTAR, SIM, SEQALN, NEEDLE, SSEARCH, and the like.

As used herein, the term “cultivate” means to grow a plant. A cultivated plant can be one grown and raised on a large agricultural scale or on a smaller scale, including for example a single plant.

As used herein, the term “hydroponic”, “hydroponics”, or “hydroponically” refers to a method of cultivating plants in the absence of soil.

Where a term is provided in the singular, other embodiments described by the plural of that term are also provided.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

### FURTHER DESCRIPTION

Isolated Methylobacterium strains that enhance early growth of plants, improve propagation/transplant vigor, increase nutrient uptake, improve stand establishment, improve stress tolerance, and/or increase a plant's ability to utilize nutrients, and compositions useful for treatment of plants with such strains are provided herein. In some embodiments, early growth enhancement results in increased yield at harvest, for example increased harvested seed yield. In certain embodiments, the Methylobacterium in the composition is selected from the group consisting of NLS0665 (NRRL B-68194), NLS0754 (NRRL B-68197), NLS0672 (NRRL B-68196), NLS0693 (NRRL B-67926), NLS0729 (NRRL B-68195), NLS0049 (NRRL B-68236), NLS0591 (NRRL B-68215), NLS0439 (NRRL B-68216), NLS1310, NLS1312, NLS0612 (NRRL B-68237), NLS0706 (NRRL B-68238), NLS0725 (NRRL B-68239), LGP2001 (NRRL B-50930), LGP2002 (NRRL B-50931), LGP2009 (NRRL B-50938), LGP2015 (NRRL B-67340), LGP2016 (NRRL B-67341), LGP2017 (NRRL B-67741), LGP2018 (NRRL B-67742), LGP2019 (NRRL B-67743), LGP2020 (NRRL B-67892), LGP2021 (NRRL B-68032), LGP2022 (NRRL B-68033), LGP2023 (NRRL B-68034), LGP2029 (NRRL B-68065), LGP2030 (NRRL B-68066), LGP2031 (NRRL B-68067), LGP2033 (NRRL B-68068), LGP2034 (NRRL B-68069), and LGP2167 (NRRL B-67927).

In certain embodiments, the Methylobacterium in the composition comprises a variant of NLS0665 (NRRL B-68194), NLS0754 (NRRL B-68197), NLS0672 (NRRL B-68196), NLS0693 (NRRL B-67926), NLS0729 (NRRL B-68195), NLS0049 (NRRL B-68236), NLS0591 (NRRL B-68215), NLS0439 (NRRL B-68216), NLS1310, NLS1312, NLS0612 (NRRL B-68237), NLS0706 (NRRL B-68238), NLS0725 (NRRL B-68239), LGP2001 (NRRL B-50930), LGP2002 (NRRL B-50931), LGP2009 (NRRL B-50938), LGP2015 (NRRL B-67340), LGP2016 (NRRL B-67341), LGP2017 (NRRL B-67741), LGP2018 (NRRL B-67742), LGP2019 (NRRL B-67743), LGP2020 (NRRL B-67892), LGP2021 (NRRL B-68032), LGP2022 (NRRL B-68033), LGP2023 (NRRL B-68034), LGP2029 (NRRL B-68065), LGP2030 (NRRL B-68066), LGP2031 (NRRL B-68067), LGP2033 (NRRL B-68068), LGP2034 (NRRL B-68069), or LGP2167 (NRRL B-67927). As noted, variants of Methylobacterium LGP2002 (NRRL B-50931), LGP2001 (NRRL B-50930), LGP2015 (NRRL B-67340), LGP2021 (NRRL B-68032), LGP2020 (NRRL B-67892), LGP2017 (NRRL B-67741), LGP2018 (NRRL B-67742), LGP2029 (NRRL B-68065), LGP2030 (NRRL B-68066), LGP2019 (NRRL B-67743), LGP2031 (NRRL B-68067), LGP2016 (NRRL B-67341), LGP2033 (NRRL B-68068), LGP2034 (NRRL B-68069), LGP2022 (NRRL B-68033), LGP2023 (NRRL B-68034), LGP2167 (NRRL B-67927), NLS1310, NLS0612 (NRRL B-68237), NLS1312, NLSO706 (NRRL B-68238), NLS0725 (NRRL B-68239), NLS0665, NLS0729, NLS0672, NLS0754, NLS0591 (NRRL B-68215), NLS0439 (NRRL B-68216), NLS0049 (NRRL B-68236), and NLS0693 (NRRL B-67926), include, for example, Methylobacterium that include at least one gene encoding a 16S RNA that has at least 97%, 98%, 99%, 99.5%, or 100% sequence identity to SEQ ID NOS: 91-120, respectively, or Methylobacterium that comprise a marker sequence with at least 97%, 98%, 99%, 99.5%, or 100% sequence identity to SEQ ID NOS: 121-131.

In certain embodiments, early plant development is enhanced, for example prior to a plant reaching the two true leaf stage. In certain embodiments, the plants are fruit or vegetable plants. In certain embodiments, the plants are leafy green plants. In certain embodiments, the plants are grown in a greenhouse. In certain embodiments, the plants are grown hydroponically or in an aeroponic plant cultivation system. Also provided is an isolated Methylobacterium strain selected from LGP2021 (NRRL B-68032), LGP2022 (NRRL B-68033), LGP2023 (NRRL B-68034), LGP2029 (NRRL B-68065), LGP2030 (NRRL B-68066), LGP2031 (NRRL B-68067), LGP2033 (NRRL B-68068), and LGP2034 (NRRL B-68069).

Further provided are methods of improving production of plants including leafy green plants, fruit and vegetable plants, rice, row crops, such as corn, soybean, wheat, barley, and such, and specialty crops, including cannabis crops, by treatment with one or more Methylobacterium strains provided herein. In some embodiments, production is improved by enhanced early growth of treated plants or plants grown from treated seeds in comparison to an untreated control plant or in comparison to a control plant grown from an untreated seed. Such enhanced early growth is measured, for example, by an increase in biomass of treated plants, including increased shoot, leaf, root, or whole seedling biomass. Increased early growth can result in various improvements in plant production, including for example increased biomass production or yield of harvested plants, increased and/or more uniform fruit production, faster seed set, earlier maturation, increased rate of leaf growth, increased rate of root growth, increased seed yield, and decreased cycle time in comparison to an untreated control plant or in comparison to a control plant grown from an untreated seed. In certain embodiments, application of Methylobacterium strains as provided herein provides for a 1%,2%, 3%,4%, 5%, 6%, 7%, 8%, 9%10%, 12%, 15%, 17%, 20%, 30%, or 40% increase in any of the aforementioned traits in comparison to an untreated control plant or in comparison to a control plant grown from an untreated seed. In some embodiments, production is enhanced by increased rooting, for example of plant cuttings, where such increased rooting can result in decreased cycling time and/or increased biomass or yield of the treated plants.

Various methods for identifying a Methylobacterium strain that enhances plant nitrogen use efficiency are also provided herein. In one method, a plant, plant part, or seed is treated with at least a first Methylobacterium strain to obtain a treated seed and/or a treated plant or plant part. Following cultivation of the plant to at least the two true leaf stage, the plant or one or more plant parts is harvested from the cultivated plant and from a control plant grown from an untreated control seed or untreated control plant, or from a plant treated with a second Methylobacterium strain. The biomass of the treated and control plant or plant parts are assayed to i) measure growth, for example by measuring root length or biomass and/or shoot biomass, and/or ii) to measure nitrogen content, for example shoot nitrogen content. In some embodiments, nitrogen levels provided to the treated plants or plant parts are reduced from levels normally considered optimal for growth of the plant. In some embodiments, Methylobacterium isolates selected for testing in such methods comprise one or more genetic elements correlated with enhanced early plant growth as further described here and exemplified for early growth or rice. In some embodiments, the first Methylobacterium isolate comprises a genetic element encoding a protein having a consensus amino acid sequence selected from the group consisting of SEQ ID NO: 77 to SEQ ID NO: 83. In some embodiments, the at least a first Methylobacterium strain comprises two or more different Methylobacterium isolates. In some embodiments, the plant is cultivated in a hydroponic or aeroponic system. In some embodiments, Methylobacterium isolates selected for testing for enhanced nitrogen use efficiency comprise one or more genetic elements encoding proteins involved in production of indole acetic acid (IAA), 1-aminocyclopropane-1-carboxylate (ACC) deaminase, and/or siderophores.

In this manner, a Methylobacterium strain or strains is identified and selected, wherein the strain provides for enhanced nitrogen use efficiency in the cultivated plant or a plant part of the cultivated plant in comparison to an untreated control plant or plant part or in comparison to plants treated with other Methylobacterium strains when grown in nitrogen limited conditions. In some embodiments, enhanced nitrogen use efficiency is evidenced by enhanced growth and/or enhanced nitrogen content in plants or plant parts. In some embodiments, a rice seed is treated. In other embodiments, a leafy green plant seed, seedling, or part thereof is treated. In some embodiments, plants, seeds, or seedlings are separately treated with two, three, four, or more Methylobacterium strains and growth and nitrogen content are compared for plants or plant parts treated with different strains, and a Methylobacterium strain or strains demonstrating increased nitrogen content and/or increased growth under nitrogen limited conditions is selected and identified as providing for enhanced nitrogen use efficiency. In other embodiments, Methylobacterium strains are applied to seeds for planting and plants grown under nitrogen limited conditions are harvested to determine effect of the strain on plant yield.

In some embodiments, increased seedling root and shoot growth resulting from treatment with Methylobacterium may contribute to enhanced nitrogen use efficiency. Thus, identification of genetic elements and encoded proteins that contribute to such enhanced plant growth can be useful for identification of strains having the ability to improve nutrient uptake and utilization, and increase nitrogen use efficiency. Genetic elements and encoded proteins correlated with enhanced plant growth described herein were identified by screening a population of Methylobacterium strains and identifying strains that enhance plant growth (hits) and strains which lack the ability to enhance growth of the tested plant (non-hits).

A genome-wide association study, or whole genome association study was performed to identify genetic elements correlated with enhanced root and shoot growth. As described herein, a pan-genome was generated (Page et al. (Bioinformatics (2015) 31:3691-3693) for the tested Methylobacterium population and hundreds of additional Methylobacterium strains collected from various locations in the United States. Using the pan-genome as a reference, the presence or absence of each genetic element in the “hit” set of strains (plant growth promoting) and the “non-hit” set of strains was determined. The presence and absence scores were used in a correlation analysis to identify the genetic elements that correlate positively with enhanced plant growth. Correlation was established using a statistical significance threshold based on empirical p-value where a cutoff of p less than or equal to 0.05 or p less than or equal to 0.10 is used. Scores for sensitivity, where the presence of the gene is used as a determination that a strain enhances plant growth, and/or specificity, where the non-presence or absence of the gene is used as an indicator that a strain did not promote growth of the tested plant, were also used in the correlation analysis.

In some embodiments, presence of a genetic element associated with enhanced seedling and root growth is detected where a genetic element in a Methylobacterium strain encodes a protein having at least 50%, 60%, 70%, 75%, 80%, 85%, 90%, or 95% sequence identity or more to a protein encoded by a genetic element correlated with promoting plant growth. In certain embodiments, the genetic element comprises a gene that encodes a protein having at least 50%, 60%, 70%, 75%, 80%, 85%, 90%, or 95% sequence identity or one or more consensus proteins having an amino acid sequence of SEQ ID NO: 77 to SEQ ID NO: 83. In some embodiments, the genetic element comprises a gene that encodes a protein having at least 50%, 60%, 70%, 75%, 80%, 85%, 90%, or 95% sequence identity or one or more representative sequences of SEQ ID NO: 84 to SEQ ID NO: 90, where the representative sequences are from strains demonstrated herein to promote early plant growth. In some cases, identity to a representative or consensus sequence may be less than 50%, for example, 40% or even 30%. In certain embodiments, the genetic element comprises a gene that encodes a protein having 30% to 50% sequence identity to a protein encoded by SEQ ID NO: 84 to SEQ ID NO: 90.

Also provided herein are methods of enhancing growth and/or yield of a plant, comprising treating a plant or soil where said a plant is growing or will be grown, with a Methylobacterium isolate that enhances uptake and/or utilization of one or more nutrient components of a fertilizer that is applied to improve cultivation of said plant. In some embodiments the one or more nutrient components is selected from the group consisting of nitrogen, phosphorus, potassium, and iron. In some embodiments, the Methylobacterium isolate is selected from the group consisting of NLS0665 (NRRL B-68194), NLS0754 (NRRL B-68197), NLS0672 (NRRL B-68196), NLS0693 (NRRL B-67926), NLS0729 (NRRL B-68195), NLS0049 (NRRL B-68236), NLS0591 (NRRL B-68215), NLS0439 (NRRL B-68216), NLS1310, NLS1312, NLS0612 (NRRL B-68237), NLS0706 (NRRL B-68238), NLS0725 (NRRL B-68239), LGP2001 (NRRL B-50930), LGP2002 (NRRL B-50931), LGP2009 (NRRL B-50938), LGP2015 (NRRL B-67340), LGP2016 (NRRL B-67341), LGP2017 (NRRL B-67741), LGP2018 (NRRL B-67742), LGP2019 (NRRL B-67743), LGP2020 (NRRL B-67892), LGP2021 (NRRL B-68032), LGP2022 (NRRL B-68033), LGP2023 (NRRL B-68034), LGP2029 (NRRL B-68065), LGP2030 (NRRL B-68066), LGP2031 (NRRL B-68067), LGP2033 (NRRL B-68068), LGP2034 (NRRL B-68069), LGP2167 (NRRL B-67927). In some embodiments, treatment with said Methylobacterium isolates allows for reduced levels of fertilizer or various fertilizer components during cultivation of said plant. In some embodiments, the plant is an agricultural row crop. In some embodiments, a Methylobacterium treated plant can be cultivated using reduced rates of fertilizer as compared to standard application rates for said plant. In some embodiments, fertilizer application can be reduced by 5%, 10%, 15%, 20%, 25%, 30%, 35%, 40%, or more. In certain embodiments, application of fertilizer can be reduced by at least 25%. In some embodiments the amount of one or more components of said fertilizer is reduced. In some embodiments levels of nitrogen, phosphorus, potassium and/or iron are reduced by 5%, 10%, 15%, 20%, 25%, 30%, 35%, 40%, or more. Optimal fertilizer and/or fertilizer components may vary depending on the crop, soil, and/or geographical location. Optimal fertilizer levels can also be determined experimentally, for example by measuring yield at increasing amounts of fertilizer, where the optimal fertilizer concentration is identified by determining the level after which no further yield advantage is observed. An example of determining the optimal nitrogen level for growth is described in Sharma et al. (Indian J Genet. (2018) 78:292-301). In some embodiments, methods for enhancing growth and/or yield of a plant comprise application of a composition comprising one or more Methylobacterium isolates selected from the group consisting of NLS0665 (NRRL B-68194), NLS0754 (NRRL B-68197), NLS0672 (NRRL B-68196), NLS0693 (NRRL B-67926), NLS0729 (NRRL B-68195), NLS0049 (NRRL B-68236), NLS0591 (NRRL B-68215), NLS0439 (NRRL B-68216), NLS1310, NLS1312, NLS0612 (NRRL B-68237), NLS0706 (NRRL B-68238), NLS0725 (NRRL B-68239), LGP2001 (NRRL B-50930), LGP2002 (NRRL B-50931), LGP2009 (NRRL B-50938), LGP2015 (NRRL B-67340), LGP2016 (NRRL B-67341), LGP2017 (NRRL B-67741), LGP2018 (NRRL B-67742), LGP2019 (NRRL B-67743), LGP2020 (NRRL B-67892), LGP2021 (NRRL B-68032), LGP2022 (NRRL B-68033), LGP2023 (NRRL B-68034), LGP2029 (NRRL B-68065), LGP2030 (NRRL B-68066), LGP2031 (NRRL B-68067), LGP2033 (NRRL B-68068), LGP2034 (NRRL B-68069), LGP2167 (NRRL B-67927), and a fertilizer. In some embodiments, the plant is an agricultural row crop. In some embodiments, the plant is a leafy green plant. In some embodiments, a leafy green plant is treated, and the leafy green plant is cultivated in a hydroponic or aeroponic plant growth environment. In some embodiments, the fertilizer or component of the fertilizer are present at a reduced rate compared to the optimal level for the plant. In some embodiments, the nitrogen level is reduced by 5%, 10%, 15%, 20%, 25%, 30%, 35%, 40%, or more.

In some embodiments of methods provided herein, a plant seed is treated. In certain other embodiments, a plant seedling or part thereof is treated. In some embodiments, a plant shoot or seedling is treated. In some embodiments, the treated plant is cultivated to the second true leaf stage (V2) and harvested to determine root and shoot biomass and nitrogen levels. In some embodiments, the treated plant is cultivated for 10 to 14 days. In some embodiments, the treated plant is cultivated for 14 to 28 days. In some embodiments, the treated plant is cultivated for 28 or more days prior to harvest and analysis of tissue samples to determine levels of nitrogen and other mineral nutrients. In some embodiments, treated plant seeds or seedlings are cultivated in a hydroponic system or an aeroponic plant growth system. A hydroponics system can be a water culture system, a nutrient film technique, an ebb and flow system, a drip system, or a wick system. In an aeroponic system, plants are grown in an air or mist environment without the use of soil. In some embodiments, the hydroponic or aeroponic system can be a variation of any of these types or a combination of one or more systems. In some embodiments, a hydroponic or aeroponic system is advantageous over a soil based cultivation system for determining effects of Methylobacterium strains due to the presence of fewer background microorganisms. Various inert substrates can be used to support the plants, seedlings, and root systems in hydroponic or aeroponic growth, including but not limited to perlite, rockwool, clay pellets, foam cubes, rock, peat moss, or vermiculite.

In some embodiments, a Methylobacterium strain that enhances plant growth or nitrogen use efficiency is more efficient at colonizing a plant host cell or tissue, as compared to other Methylobacterium strains. Methods for identifying microbial strains having enhanced colonization efficiency are described in WO2020163027 (PCT/US2020/012041), which is incorporated herein by reference in its entirety. In some embodiments, a Methylobacterium strain that increases the nitrogen use efficiency of a plant or plant part also imparts a trait improvement to said plant selected from increased biomass production, decreased cycle time, increased rate of leaf growth, decreased time to develop two true leaves, increased rate of root growth, and increased seed yield.

Various methods of using Methylobacterium strains to enhance early growth or rooting, improve propagation/transplant vigor, increase nutrient uptake, improve stand establishment, improve stress tolerance, and/or increase a plant's ability to uptake and/or utilize nutrients, such as nitrogen, potassium, sulfur, cobalt, copper, zinc, phosphorus, boron, iron, and manganese in plants, such as leafy green plants, row crops, cannabis, and other specialty crops are provided herein. In certain embodiments, Methylobacterium treatment of a row crop, including but not limited to corn, soybean, rice, canola, and wheat, results in enhanced plant growth and yield. In certain embodiments, the crop is rice and the Methylobacterium is selected from the group consisting of LGP2016 (NRRL B-67341), LGP2017 (NRRL B-67741), LGP2019 (NRRL B-67743), and variants thereof. In some embodiments, Methylobacterium selected from LGP2002 (NRRL B-50931), LGP2009 (NRRL B-50938), LGP2022 (NRRL B-68033), a combination of LGP2002 (NRRL B-50931) and LGP2015 (NRRL B-67340), and other combinations and variants thereof, are applied to rosemary, French tarragon, basil, Pennisetum, and other herbs. In certain embodiments, Methylobacterium treatment of soil, a seed, a leaf, a stem, a root, or a shoot can enhance early growth, propagation/transplant vigor, stand establishment, and/or stress tolerance as well as or alternatively enhance nutrient use efficiency. Enhanced nutrient use efficiency can result in increased levels of nitrogen and other mineral nutrients, including for example, potassium, sulfur, copper, zinc, phosphorus, boron, iron, and manganese in a treated plant. In some embodiments, Methylobacterium NLS0665 (NRRL B-68194), NLS0754 (NRRL B-68197), NLS0672 (NRRL B-68196), NLS0693 (NRRL B-67926), NLS0729 (NRRL B-68195), NLS0049 (NRRL B-68236), NLS0591 (NRRL B-68215), NLS0439 (NRRL B-68216), NLS1310, NLS1312, NLS0612 (NRRL B-68237), NLS0706 (NRRL B-68238), NLS0725 (NRRL B-68239), LGP2001 (NRRL B-50930), LGP2002 (NRRL B-50931), LGP2009 (NRRL B-50938), LGP2015 (NRRL B-67340), LGP2016 (NRRL B-67341), LGP2017 (NRRL B-67741), LGP2018 (NRRL B-67742), LGP2019 (NRRL B-67743), LGP2020 (NRRL B-67892), LGP2021 (NRRL B-68032), LGP2022 (NRRL B-68033), LGP2023 (NRRL B-68034), LGP2029 (NRRL B-68065), LGP2030 (NRRL B-68066), LGP2031 (NRRL B-68067), LGP2033 (NRRL B-68068), LGP2034 (NRRL B-68069), LGP2167 (NRRL B-67927), or variants thereof are applied to plants, plant parts, or seeds.

Alternatively, such Methylobacterium may be applied to soil or other growth medium where plants are grown. Methylobacterium soil treatments or applications can include, but are not limited to, in-furrow applications (e.g., before, during, and/or after seed deposition), soil drenches, and distribution of granular or other dried formulations to the soil (e.g., before, during, and/or after seed deposition or plant growth). Methylobacterium treatments for plants grown in hydroponic systems can include seed treatments prior to germination, foliar applications to germinated plants or parts thereof, and applications in a liquid solution used in the hydroponic system. In certain embodiments, Methylobacterium treatment of a plant can include application to the seed, plant, and/or a part of the plant and can thus comprise any Methylobacterium treatment or application resulting in colonization of the plant by the Methylobacterium. In some embodiments, application of Methylobacterium to crops that are propagated by cutting can enhance growth and/or rooting of such plants. Field transplants of such treated and rooted cuttings may demonstrate decreased cycling time and/or improved biomass and/or yield as a result of such treatments. In some embodiments Methylobacterium selected from LGP2002 (NRRL B-50931), LGP2009 (NRRL B-50938), LGP2019 (NRRL B-67743), and variants thereof are applied to cannabis cuttings to improve growth and root development.

Treatments or applications to plants described herein can include, but are not limited to, spraying, coating, partially coating, immersing, drenching, and/or imbibing the seed, plant, or plant parts with the Methylobacterium strains and compositions comprising the same provided herein. In certain embodiments, soil, a seed, a leaf, a stem, a root, a tuber, or a shoot can be sprayed, immersed, drenched and/or imbibed with a liquid, semi-liquid, emulsion, or slurry of a composition provided herein. Such treatments, applications, seed immersion, or imbibition can be sufficient to provide for enhanced early growth and/or increased levels of one or more mineral nutrients and/or vitamins content in harvestable tissue from a treated plant or plant grown from a treated seed in comparison to an untreated plant or plant grown from an untreated seed. Enhanced early growth can lead to further improvements in plant production including an increase in biomass of treated plants, such as increased shoot, root, or whole seedling biomass. Enhanced early growth can result in various additional improvements in plant production, including for example increased yield of harvested plants or harvested plant parts, increased and/or more uniform fruit production, faster seed set, earlier maturation, increased rate of leaf growth, increased rate of root growth, increased seed yield, and decreased cycle time. In certain embodiments, plant seeds or cuttings can be immersed and/or imbibed for at least 1, 2, 3, 4, 5, or 6 hours. Such immersion and/or imbibition can, in certain embodiments, be conducted at temperatures that are not deleterious to the plant seed or the Methylobacterium. In certain embodiments, the seeds can be treated at about 15 to about 30 degrees Centigrade or at about 20 to about 25 degrees Centigrade. In certain embodiments, seed imbibition and/or immersion can be performed with gentle agitation. Seed treatments can be effected with both continuous and/or batch seed treaters. In certain embodiments, the coated seeds can be prepared by slurrying seeds with a coating composition comprising a Methylobacterium strain that increases the levels of one or more mineral nutrients and/or vitamins and air-drying the resulting product. Air-drying can be accomplished at any temperature that is not deleterious to the seed or the Methylobacterium, but will typically not be greater than 30 degrees Centigrade. The proportion of coating that comprises the Methylobacterium strain includes, but is not limited to, a range of 0.1 to 25% by weight of the seed or other plant part, 0.5 to 5% by weight of the seed or other plant part, and 0.5 to 2.5% by weight of the seed or other plant part. In certain embodiments, a solid substance used in the seed coating or treatment will have a Methylobacterium strain that increases mineral nutrient and/or vitamin content adhered to a solid substance as a result of being grown in biphasic media comprising the Methylobacterium strain, solid substance, and liquid media. Methods for growing Methylobacterium in biphasic media include those described in U.S. Pat. No. 9,181,541, which is specifically incorporated herein by reference in its entirety. In certain embodiments, compositions suitable for treatment of a seed or plant part can be obtained by the methods provided in U.S. Pat. No. 10,287,544, which is specifically incorporated herein by reference in its entirety. Various seed treatment compositions and methods for seed treatment disclosed in U.S. Pat. Nos. 5,106,648, 5,512,069, and 8,181,388 are incorporated herein by reference in their entireties and can be adapted for treating seeds with compositions comprising a Methylobacterium strain.

In certain embodiments where plant seeds are treated with Methylobacterium compositions provided herein, the compositions further comprise one or more lubricants to ensure smooth flow and separation (singulation) of seeds in the seeding mechanism, for example a planter box. Lubricants for use in such compositions include talc, graphite, polyethylene wax based powders (such as Fluency Agent), protein powders, for example soybean protein powders, or a combination of protein powders and a lipid, for example lecithin or a vegetable oil. Lubricants can be applied to seeds simultaneously with application of Methylobacterium, or may be mixed with Methylobacterium prior to application of the compositions to the seeds.

In certain embodiments, treated plants are cultivated in a hydroponic system. In some embodiments, plant seeds are treated and plants are grown from the treated seeds continuously in the same cultivation system. In some embodiments, plant seeds are treated and cultivated in a hydroponic nursery to produce seedlings. The seedlings are transferred to a different hydroponic system, for example for commercial production of leafy greens. In some embodiments, a Methylobacterium strain that enhances early growth or increases the levels of one or more mineral nutrients and/or vitamins persists in the seedlings transferred to a greenhouse production system and continues to provide advantages such as improved micronutrient and/or vitamin content and/or biomass production, through the further growth of the leafy green plant. In some embodiments, plant seedlings transferred to a greenhouse production system may be further treated with NLS0665 (NRRL B-68194), NLS0754 (NRRL B-68197), NLS0672 (NRRL B-68196), NLS0693 (NRRL B-67926), NLS0729 (NRRL B-68195), NLS0049 (NRRL B-68236), NLS0591 (NRRL B-68215), NLS0439 (NRRL B-68216), NLS1310, NLS1312, NLS0612 (NRRL B-68237), NLS0706 (NRRL B-68238), NLS0725 (NRRL B-68239), LGP2001 (NRRL B-50930), LGP2002 (NRRL B-50931), LGP2009 (NRRL B-50938), LGP2015 (NRRL B-67340), LGP2016 (NRRL B-67341), LGP2017 (NRRL B-67741), LGP2018 (NRRL B-67742), LGP2019 (NRRL B-67743), LGP2020 (NRRL B-67892), LGP2021 (NRRL B-68032), LGP2022 (NRRL B-68033), LGP2023 (NRRL B-68034), LGP2029 (NRRL B-68065), LGP2030 (NRRL B-68066), LGP2031 (NRRL B-68067), LGP2033 (NRRL B-68068), LGP2034 (NRRL B-68069), LGP2167 (NRRL B-67927), or variants thereof, or with one or more other Methylobacterium strains that increase the levels of one or more mineral nutrients and/or vitamins prior to, during, or after transfer to the production system.

In certain embodiments, the composition used to treat the seed or plant part can contain a Methylobacterium strain and an agriculturally acceptable excipient. Agriculturally acceptable excipients include, but are not limited to, woodflours, clays, activated carbon, diatomaceous earth, fine-grain inorganic solids, calcium carbonate, and the like. Clays and inorganic solids that can be used include, but are not limited to, calcium bentonite, kaolin, china clay, talc, perlite, mica, vermiculite, silicas, quartz powder, montmorillonite, and mixtures thereof. Agriculturally acceptable excipients also include various lubricants such as talc, graphite, polyethylene wax based powders (such as Fluency Agent), protein powders, for example soybean protein powders, or a combination of protein powders and a lipid, for example lecithin or a vegetable oil.

Agriculturally acceptable adjuvants that promote sticking to the seed that can be used include, but are not limited to, polyvinyl acetates, polyvinyl acetate copolymers, hydrolyzed polyvinyl acetates, polyvinylpyrrolidone-vinyl acetate copolymer, polyvinyl alcohols, polyvinyl alcohol copolymers, polyvinyl methyl ether, polyvinyl methyl ether-maleic anhydride copolymer, waxes, latex polymers, celluloses including ethylcelluloses and methylcelluloses, hydroxy methylcelluloses, hydroxypropylcellulose, hydroxymethylpropylcelluloses, polyvinyl pyrrolidones, alginates, dextrins, malto-dextrins, polysaccharides, fats, oils, proteins, karaya gum, jaguar gum, tragacanth gum, polysaccharide gums, mucilage, gum arabics, shellacs, vinylidene chloride polymers and copolymers, soybean-based protein polymers and copolymers, lignosulfonates, acrylic copolymers, starches, polyvinylacrylates, zeins, gelatin, carboxymethylcellulose, chitosan, polyethylene oxide, acrylamide polymers and copolymers, polyhydroxyethyl acrylate, methylacrylamide monomers, alginate, ethylcellulose, polychloroprene, and syrups or mixtures thereof. Other useful agriculturally acceptable adjuvants that can promote coating include, but are not limited to, polymers and copolymers of vinyl acetate, polyvinylpyrrolidone-vinyl acetate copolymer, and water-soluble waxes. Further, agriculturally acceptable adjuvants also include various lubricants (which can provide for smooth flow and separation (singulation) of seeds) such as talc, graphite, polyethylene wax based powders (such as Fluency Agent), protein powders, for example soybean protein powders, or a combination of protein powders and a lipid, for example lecithin or a vegetable oil. Various surfactants, dispersants, anticaking-agents, foam-control agents, and dyes disclosed herein and in U.S. Pat. No. 8,181,388 can be adapted for use with compositions comprising a suitable Methylobacterium strain. In certain embodiments, the seed and/or seedling is exposed to the composition by providing the Methylobacterium strain in soil in which the plant or a plant arising from the seed are grown, or other plant growth media in which the plant or a plant arising from the seed are grown. Examples of methods where the Methylobacterium strain is provided in the soil include in furrow applications, soil drenches, and the like.

Non-limiting examples of treatments of plant seeds, seedling, or other plant parts with a Methylobacterium providing for enhanced early growth and/or increased content of one or more mineral nutrients and/or vitamins in a harvested plant part include treatments of vegetable crops with edible leaves including, without limitation, spinach, kale, lettuce (including but not limited to romaine, butterhead, iceberg and loose leaf lettuces), and field greens, including brassica greens. Specific greens that can be treated with Methylobacterium provided herein include collard greens, cabbage, beet greens, watercress, swiss chard, arugula, escarole, endive, bok choy, and turnip greens. Other leafy green plants that are grown for production and harvest of microgreens and/or herbs, can also be treated in the methods described herein to provide for increased content of one or more mineral nutrients and/or vitamins in harvested microgreens, including but not limited to lettuce, cauliflower, broccoli, cabbage, watercress, arugula, garlic, onion, leek, amaranth, swill chard, been, spinach, melon, cucumber, squash, basil, celery, cilantro, radish, radicchio, chicory, dill, rosemary, French tarragon, basil, Pennisetum, carrot, fennel, beans, peas, chickpeas, and lentils. Treatment of plants grown for harvest of fleshy fruits are also provided herein. Such plants include, for example, melon (including watermelon and cantaloupe), berry (including strawberry, blueberry, blackberry, and raspberry), grape, kiwi, mango, papaya, pineapple, banana, pepper, tomato, squash, and cucumber plants.

In certain embodiments, NLS0665 (NRRL B-68194), NLS0754 (NRRL B-68197), NLS0672 (NRRL B-68196), NLS0693 (NRRL B-67926), NLS0729 (NRRL B-68195), NLS0049 (NRRL B-68236), NLS0591 (NRRL B-68215), NLS0439 (NRRL B-68216), NLS1310, NLS1312, NLS0612 (NRRL B-68237), NLS0706 (NRRL B-68238), NLS0725 (NRRL B-68239), LGP2001 (NRRL B-50930), LGP2002 (NRRL B-50931), LGP2009 (NRRL B-50938), LGP2015 (NRRL B-67340), LGP2016 (NRRL B-67341), LGP2017 (NRRL B-67741), LGP2018 (NRRL B-67742), LGP2019 (NRRL B-67743), LGP2020 (NRRL B-67892), LGP2021 (NRRL B-68032), LGP2022 (NRRL B-68033), LGP2023 (NRRL B-68034), LGP2029 (NRRL B-68065), LGP2030 (NRRL B-68066), LGP2031 (NRRL B-68067), LGP2033 (NRRL B-68068), LGP2034 (NRRL B-68069), LGP2167 (NRRL B-67927), LGP2032, LGP2024, NLS0681, NLS0594, NLS0479, NLS1310, NLS0612 (NRRL B-68237), NLS1312, NLS0473, NLS0706 (NRRL B-68238), NLS0725 (NRRL B-68239), or variants or combinations thereof will also find use in treatment of other plant species to enhance early growth, including, for example field crops, leafy greens, herbs, ornamentals, turf grasses, and trees grown in commercial production, such as conifer trees. Without limitation, such additional plant species include corn, soybean, cruciferous or Brassica sp. vegetables (e.g., B. napus, B. rapa, B. juncea), alfalfa, rice, rye, wheat, barley, oats, sorghum, millet (e.g., pearl millet (Pennisetum glaucum), proso millet (Panicum miliaceum), foxtail millet (Setaria italica), and finger millet (Eleusine coracana)), sunflower, safflower, tobacco, potato, peanuts, cotton, species in the genus Cannabis (including, but not limited to, Cannabis sativa and industrial hemp varieties), sweet potato (Ipomoea batatus), cassava, coffee, coconut, ornamentals (including, but not limited to, azalea, hydrangea, hibiscus, roses, tulips, daffodils, petunias, carnation, poinsettia, and chrysanthemum), conifers (including, but not limited to pines such as loblolly pine, slash pine, ponderosa pine, lodge pole pine, and Monterey pine; Douglas-fir; Western hemlock; Sitka spruce; redwood; true firs such as silver fir and balsam fir; and cedars such as Western red cedar and Alaska yellow-cedar), and turfgrass (including, but are not limited to, annual bluegrass, annual ryegrass, Canada bluegrass, fescue, bentgrass, wheatgrass, Kentucky bluegrass, orchard grass, ryegrass, redtop, Bermuda grass, St. Augustine grass, and zoysia grass); fruit (including but not limited to citrus, pome, and tropical fruit); nuts; and tea. Leafy green plants that can be treated include vegetable crop with edible leaves, for example, spinach, kale, lettuce (including but not limited to romaine, butterhead, iceberg and loose leaf lettuces), collard greens, cabbage, beet greens, watercress, swiss chard, arugula, escarole, endive, bok choy and turnip greens. Leafy green plants as used herein also refers to plants grown for harvest of microgreens and/or herbs, including but not limited to lettuce, cauliflower, broccoli, cabbage, watercress, arugula, garlic, onion, leek, amaranth, swill chard, been, spinach, melon, cucumber, squash, basil, celery, cilantro, radish, radicchio, chicory, dill, rosemary, French tarragon, basil, Pennisetum, carrot, fennel, beans, peas, chickpeas, and lentils.

In certain embodiments, a Methylobacterium strain used to treat a given cultivar or variety of plant seed, plant, or plant part can be a Methylobacterium strain that was isolated from a different plant species, or a different cultivar or variety of the plant species being treated, and is thus heterologous or non-resident to the treated plant or plant part. Plant parts that have increased levels of one or more mineral nutrients and/or vitamins as the result of treatment with Methylobacterium as provided herein include, but are not limited to, leaves, stems, flowers, roots, seeds, fruit, tubers, coleoptiles, and the like. In certain embodiments, a plant having enhanced early growth as a result of treatment with NLS0665 (NRRL B-68194), NLS0754 (NRRL B-68197), NLS0672 (NRRL B-68196), NLS0693 (NRRL B-67926), NLS0729 (NRRL B-68195), NLS0049 (NRRL B-68236), NLS0591 (NRRL B-68215), NLS0439 (NRRL B-68216), NLS1310, NLS1312, NLS0612 (NRRL B-68237), NLS0706 (NRRL B-68238), NLS0725 (NRRL B-68239), LGP2001 (NRRL B-50930), LGP2002 (NRRL B-50931), LGP2009 (NRRL B-50938), LGP2015 (NRRL B-67340), LGP2016 (NRRL B-67341), LGP2017 (NRRL B-67741), LGP2018 (NRRL B-67742), LGP2019 (NRRL B-67743), LGP2020 (NRRL B-67892), LGP2021 (NRRL B-68032), LGP2022 (NRRL B-68033), LGP2023 (NRRL B-68034), LGP2029 (NRRL B-68065), LGP2030 (NRRL B-68066), LGP2031 (NRRL B-68067), LGP2033 (NRRL B-68068), LGP2034 (NRRL B-68069), LGP2167 (NRRL B-67927), or variants thereof, or a plant having enhanced levels of one or more mineral nutrients as a results of treatment with Methylobacterium compositions provided herein is a leafy green plant. In some embodiments, a plant having enhanced early growth as a result of treatment with a Methylobacterium provided herein, or a plant having enhanced levels of one or more mineral nutrients as a results of treatment with Methylobacterium compositions provided herein is an agricultural row crop plant. In some embodiments, increased levels of one or more mineral nutrients and/or vitamins are present in a leaf. In certain embodiments, the increased levels of one or more mineral nutrients and/or vitamins are present in the harvested greens, including leaves and shoots.

In certain embodiments, a manufactured combination composition comprising two or more Methylobacterium strains can be used to treat a seed or plant part in any of the methods provided herein. Such manufactured combination compositions can be made by methods that include harvesting monocultures of each Methylobacterium strain and mixing the harvested monocultures to obtain the manufactured combination composition of Methylobacterium. In certain embodiments, the manufactured combination composition of Methylobacterium can comprise Methylobacterium isolated from different plant species or from different cultivars or varieties of a given plant.

In certain embodiments, an effective amount of the Methylobacterium strain or strains used in treatment of plants, seeds, or plant parts is a composition having a Methylobacterium titer of at least about 1×10⁶ colony-forming units per milliliter, at least about 5×10⁶ colony-forming units per milliliter, at least about 1×10⁷ colony-forming units per milliliter, at least about 5×10⁸ colony-forming units per milliliter, at least about 1×10⁹ colony-forming units per milliliter, at least about 1×10¹⁰ colony-forming units per milliliter, or at least about 3×10¹⁰ colony-forming units per milliliter. In certain embodiments, an effective amount of the Methylobacterium strain or strains is a composition with the Methylobacterium at a titer of about least about 1×10⁶ colony-forming units per milliliter, at least about 5×10⁶ colony-forming units per milliliter, at least about 1×10⁷ colony-forming units per milliliter, or at least about 5×10⁸ colony-forming units per milliliter to at least about 6×10¹⁰ colony-forming units per milliliter of a liquid or an emulsion. In certain embodiments, an effective amount of the Methylobacterium strain or strains is a composition with the Methylobacterium at least about 1×10⁶ colony-forming units per gram, at least about 5×10⁶ colony-forming units per gram, at least about 1×10⁷ colony-forming units per gram, or at least about 5×10⁸ colony-forming units per gram to at least about 6×10¹⁰ colony-forming units of Methylobacterium per gram of the composition. In certain embodiments, an effective amount of a composition provided herein can be a composition with a Methylobacterium titer of at least about 1×10⁶ colony-forming units per gram, at least about 5×10⁶ colony-forming units per gram, at least about 1×10⁷ colony-forming units per gram, or at least about 5×10⁸ colony-forming units per gram to at least about 6×10¹⁰ colony-forming units of Methylobacterium per gram of particles in the composition containing the particles that comprise a solid substance wherein a mono-culture or co-culture of Methylobacterium strain or strains is adhered thereto. In certain embodiments, an effective amount of a composition provided herein to a plant or plant part can be a composition with a Methylobacterium titer of at least about 1×10⁶ colony-forming units per mL, at least about 5×10⁶ colony-forming units per mL, at least about 1×10⁷ colony-forming units per mL, or at least about 5×10⁸ colony-forming units per mL to at least about 6×10¹⁰ colony-forming units of Methylobacterium per mL in a composition comprising an emulsion wherein a mono-culture or co-culture of a Methylobacterium strain or strains adhered to a solid substance is provided therein or grown therein. In certain embodiments, an effective amount of a composition provided herein can be a composition with a Methylobacterium titer of at least about 1×10⁶ colony-forming units per mL, at least about 5×10⁶ colony-forming units per mL, at least about 1×10⁷ colony-forming units per mL, or at least about 5×10⁸ colony-forming units per mL to at least about 6×10¹⁰ colony-forming units of Methylobacterium per mL in a composition comprising an emulsion wherein a mono-culture or co-culture of a Methylobacterium strain or strains is provided therein or grown therein. In certain embodiments, any of the aforementioned compositions comprising a mono-culture or co-culture of a Methylobacterium strain or strains can further comprise a mono- or co-culture of Rhizobium and/or Bradyrhizobium.

In certain embodiments, an effective amount of a Methylobacterium strain or strains that provides for increased early growth and/or increased mineral nutrient and/or vitamin content provided in a treatment of a seed or plant part is at least about 10³, 10⁴, 10⁵, or 10⁶ CFU per seed or treated plant part. In certain embodiments, an effective amount of Methylobacterium provided in a treatment of a seed or plant part is at least about 10³, 10⁴, 105, or 10⁶ cfu to about 10⁷, 10⁸, 10⁹, or 10¹⁰ cfu per seed or treated plant part. in certain embodiments, the effective amount of Methylobacterium provided in a treatment of a seed or plant part is an amount where the CFU per seed or treated plant part will exceed the number of CFU of any resident naturally occurring Methylobacterium strain by at least 5-, 10-, 100-, or 1000-fold. In certain embodiments, the effective amount of Methylobacterium provided in a treatment of a seed or plant part is an amount where the CFU per seed or treated plant part will exceed the number of CFU of any resident naturally occurring Methylobacterium by at least 2—, 3-, 5-, 8-, 10-, 20-, 50-, 100-, or 1000-fold. In certain embodiments where the treated plant is cultivated in a hydroponic system, populations of naturally occurring Methylobacterium or other soil microbes will be minimal.

Non-limiting examples of Methylobacterium strains that can be used in methods provided herein are disclosed in Table 1. Other Methylobacterium strains useful in certain methods provided herein include variants of the Methylobacterium strains disclosed in Table 1. Also of use are various combinations of two or more strains or variants of Methylobacterium strains disclosed in Table 1 for treatment of plants or parts thereof.

TABLE 1
Methylobacterium sp. strain

| | | USDA ARS | |
|---|---|---|---|
| Deposit Identifier | LGP NO. | NRRL No.¹ | Strain Source: Obtained from: |
| Methylobacterium sp. #1 | LGP2000 | NRRL B-50929 | A soybean plant grown in Saint Louis |
| | | | County, Missouri, USA |
| Methylobacterium sp. #2 | LGP2001 | NRRL B-50930 | A weed grown in Saint Louis County, |
| | | | Missouri, USA |
| Methylobacterium sp. #3 | LGP2002 | NRRL B-50931 | A mint plant grown in Saint Louis County, |
| | | | Missouri, USA |
| Methylobacterium sp. #4 | LGP2003 | NRRL B-50932 | A soybean plant grown in Saint Louis |
| | | | County, Missouri, USA |
| Methylobacterium sp. #5 | LGP2004 | NRRL B-50933 | A broccoli plant grown in Saint Louis |
| | | | County, Missouri, USA |
| Methylobacterium sp. #6 | LGP2005 | NRRL B-50934 | A corn plant grown in Saint Louis County, |
| | | | Missouri, USA |
| Methylobacterium sp. #7 | LGP2006 | NRRL B-50935 | A corn plant grown in Saint Louis County, |
| | | | Missouri, USA |
| Methylobacterium sp. #8 | LGP2007 | NRRL B-50936 | A corn plant grown in Saint Louis County, |
| | | | Missouri, USA |
| Methylobacterium sp. #9 | LGP2008 | NRRL B-50937 | A corn plant grown in Saint Louis County, |
| | | | Missouri, USA |
| Methylobacterium sp. #10 | LGP2009 | NRRL B-50938 | A corn plant grown in Saint Louis County, |
| | | | Missouri, USA |
| Methylobacterium sp. #11 | LGP2010 | NRRL B-50939 | A lettuce plant grown in Saint Louis |
| | | | County, Missouri, USA |
| Methylobacterium sp. #12 | LGP2011 | NRRL B-50940 | A corn plant grown in Saint Louis County, |
| | | | Missouri, USA |
| Methylobacterium sp. #13 | LGP2012 | NRRL B-50941 | A tomato plant grown in Saint Louis |
| | | | County, Missouri, USA |
| Methylobacterium sp. #14 | LGP2013 | NRRL B-50942 | A tomato plant grown in Saint Louis |
| | | | County, Missouri, USA |
| Methylobacterium sp. #15 | LGP2014 | NRRL B-67339 | A soybean plant grown in Saint Louis |
| | | | County, Missouri, USA |
| Methylobacterium sp. #16 | LGP2015 | NRRL B-67340 | A yucca plant grown in Saint Louis |
| | | | County, Missouri, USA |
| Methylobacterium sp. #17 | LGP2016 | NRRL B-67341 | A soybean plant grown in Saint Louis |
| | | | County, Missouri, USA |
| Methylobacterium sp. #18 | LGP2017 | NRRL B-67741 | A Dionaea muscipula plant (Venus fly trap) |
| | | | grown in St. Charles, MO. |
| Methylobacterium sp. #19 | LGP2018 | NRRL B-67742 | An Orchidaceae spp. plant (orchid) grown |
| | | | in Saint Louis County, Missouri, USA |
| Methylobacterium sp. #20 | LGP2019 | NRRL B-67743 | A tomato plant grown in Saint Louis |
| | | | County, Missouri, USA |
| Methylobacterium sp. #22 | NLS0497 | NRRL B-67925 | A cup plant (Silphium perfoliatum in |
| | | | Sappington, MO) |
| Methylobacterium sp. #23 | NLS0693 | NRRL B-67926 | a vinca vine (Vinca minor) in Saint Louis |
| | | | County, Missouri, USA |
| Methylobacterium sp. #24 | NLS1179 | NRRL B-67929 | Rainwater collected in Saint Louis |
| | | | County, Missouri, USA |
| Methylobacterium sp. #25 | LGP2167 | NRRL B-67927 | An Acer ginnala (Amur maple) grown in |
| | | | Saint Louis County, Missouri, USA |
| Methylobacterium sp. #26 | LGP2020 | NRRL B-67892 | A Lagerstroemia indica (crape myrtle) |
| | | | plant grown in Saint Louis County, |
| | | | Missouri, USA |
| Methylobacterium sp. #28 | LGP2021 | NRRL B-68032 | A Cichorium intybus (chicory) plant |
| | | | growing in Saint Louis County, Missouri, |
| | | | USA |
| Methylobacterium sp. #29 | LGP2022 | NRRL B-68033 | A Coronilla vario (crown vetch) plant |
| | | | growing in Saint Louis County, Missouri, |
| | | | USA |
| Methylobacterium sp. #30 | LGP2023 | NRRL B-68034 | A Catharanthus roseus (periwinkle) |
| | | | growing in Fort Myers, Florida, USA |
| Methylobacterium sp. #31 | LGP2028 | NRRL B-68064 | A Nasturtium spp. growing in Saint Louis |
| | | | County, Missouri, USA |
| Methylobacterium sp #32 | LGP2029 | NRRL B-68065 | A Salvia officinalis (sage) growing in Saint |
| | | | Louis County, Missouri, USA |
| Methylobacterium sp #33 | LGP2030 | NRRL B-68066 | A Prunus persica (peach, ‘Hale Haven’), |
| | | | growing in Dudley, Missouri, USA |
| Methylobacterium sp #34 | LGP2031 | NRRL B-68067 | An Acer spp. (maple) growing in Dudley, |
| | | | Missouri, USA |
| Methylobacterium sp #35 | LGP2033 | NRRL B-68068 | A Rosa rugosa (Japanese rose) growing in |
| | | | Camden, Maine, USA |
| Methylobacterium sp #36 | LGP2034 | NRRL B-68069 | A Solidago sp. (goldenrod) growing in |
| | | | Camden, Maine, USA |
| Methylobacterium sp #43 | NLS0665 | NRRL B-68194 | An orchid (Orchidaceae spp.) growing in |
| | | | Saint Louis County, Missouri, USA |
| Methylobacterium sp #44 | NLS0729 | NRRL B-68195 | A yellow rose (Rosa spp.) growing in Saint |
| | | | Louis County, Missouri, USA |
| Methylobacterium sp #45 | NLS0672 | NRRL B-68196 | A rosemary plant (Rosmarinus officianalis) |
| | | | growing in Saint Louis County, Missouri, |
| | | | USA |
| Methylobacterium sp #46 | NLS0754 | NRRL B-68197 | A corn plant (Zea mays) grown in Farmer |
| | | | city, Illinois, USA |
| Methylobacterium sp #47 | NLS0591 | NRRL B-68215 | A wild grape vine (Vinis spp.) growing in |
| | | | Saint Louis County, Missouri, USA |
| Methylobacterium sp #48 | NLS0439 | NRRL B-68216 | A hairy-leaved sedge (Carex hirustella) |
| | | | plant growing in Saint Louis County, |
| | | | Missouri, USA |
| Methylobacterium sp #49 | NLS1310 | NRRL B-68217 | A blackberry plant (Rubus spp.) growing in |
| | | | Saint Louis County, Missouri, USA |
| Methylobacterium sp #50 | NLS1312 | NRRL B-68218 | A blackberry plant (Rubus spp.) growing in |
| | | | Saint Louis County, Missouri, USA |
| Methylobacterium sp #51 | NLS0049 | NRRL B-68236 | A soybean plant grown in Saint Louis |
| | | | County, Missouri, USA |
| Methylobacterium sp #52 | NLS0612 | NRRL B-68237 | A crape myrtle plant (Lagerstroemia |
| | | | indica) growing in Saint Louis County, |
| | | | Missouri, USA |
| Methylobacterium sp #53 | NLS0706 | NRRL B-68238 | A dill plant (Anethum graveolens) growing |
| | | | in Saint Louis County, Missouri, USA |
| Methylobacterium sp #54 | NLS0725 | NRRL B-68239 | A blackberry plant (Rubus spp.) growing in |
| | | | Saint Louis County, Missouri, USA |

¹Deposit number for strain deposited with the AGRICULTURAL RESEARCH SERVICE CULTURE COLLECTION (NRRL) of the National Center for Agricultural Utilization Research, Agricultural Research Service, U.S. Department of Agriculture, 1815 North University Street, Peoria, Illinois 61604 U.S.A. under the terms of the Budapest Treaty on the International Recognition of the Deposit of Microorganisms for the Purposes of Patent Procedure. Subject to 37 CFR §1.808(b), all restrictions imposed by the depositor on the availability to the public of the deposited material will be irrevocably removed upon the granting of any patent from this patent application.

Variants of a Methylobacterium isolate listed in Table 1 include isolates obtained therefrom by genetic transformation, mutagenesis, and/or insertion of a heterologous sequence. In some embodiments, such variants are identified by the presence of chromosomal genomic DNA with at least 99%, 99.9%, 99.8%, 99.7%, 99.6%, or 99.5% sequence identity to chromosomal genomic DNA of the strain from which it was derived. In certain embodiments, such variants are distinguished by the presence of one or more unique DNA sequences that include: (i) a unique sequence of SEQ ID NOs: 1 to 3, SEQ ID NOs: 13 to 15, SEQ ID NOs: 25 to 27, SEQ ID NOs: 37 to 39, SEQ ID NOs: 49 to 51, and SEQ ID NOs: 61 to 73; or (ii) sequences with at least 98% or 99% sequence identity across the full length of SEQ ID NOs: 1 to 3, SEQ ID NOs: 13 to 15, SEQ ID NOs: 25 to 27, SEQ ID NOs: 37 to 39, SEQ ID NOs: 49 to 51, SEQ ID NOs: 61 to 73, and SEQ ID NOs: 74 to 76.

In certain embodiments of the methods provided herein, the Methylobacterium strain or strains used to treat a plant, plant part, and/or seed are selected from the group consisting of LGP2032, LGP2024, NLS0681, NLS0594, NLS0479, NLS1310, NLS0612 (NRRL B-68237), NLS1312, NLS0473, NLS0706 (NRRL B-68238), NLS0725 (NRRL B-68239), LGP2000 (NRRL B-50929), LGP2001 (NRRL B-50930), LGP2002 (NRRL B-50931), LGP2003 (NRRL B-50932), LGP2004 (NRRL B-50933), LGP2005 (NRRL B-50934), LGP2006 (NRRL B-50935), LGP2007 (NRRL B-50936), LGP2008 (NRRL B-50937), LGP2009 (NRRL B-50938), LGP2010 (NRRL B-50939), LGP2011 (NRRL B-50940), LGP2012 (NRRL B-50941), LGP2013 (NRRL B-50942), LGP2014 (NRRL B-67339), LGP2015 (NRRL B-67340), LGP2016 (NRRL B-67341), LGP2017 (NRRL B-67741), LGP2018 (NRRL B-67742), LGP2019 (NRRL B-67743), LGP2020 (NRRL B-67892), LGP2021 (NRRL B-68032), LGP2022 (NRRL B-68033), LGP2023 (NRRL B-68034), LGP2029 (NRRL B-68065), LGP2030 (NRRL B-68066), LGP2031 (NRRL B-68067), LGP2033 (NRRL B-68068), LGP2034 (NRRL B-68069), LGP2167 (NRRL B-67927), variants thereof, or any combination thereof. In certain embodiments, one or more of the Methylobacterium strains used in the methods provided herein comprise total genomic DNA (chromosomal and plasmid DNA) or average nucleotide identity (ANI) with at least 99%, 99.9%, 99.8%, 99.7%, 99.6%, or 99.5% sequence identity or ANI to total genomic DNA of LGP2032, LGP2024, NLS0681, NLS0594, NLS0479, NLS1310, NLS0612 (NRRL B-68237), NLS1312, NLS0473, NLS0706 (NRRL B-68238), NLS0725 (NRRL B-68239), LGP2000 (NRRL B-50929), LGP2001 (NRRL B-50930), LGP2002 (NRRL B-50931), LGP2003 (NRRL B-50932), LGP2004 (NRRL B-50933), LGP2005 (NRRL B-50934), LGP2006 (NRRL B-50935), LGP2007 (NRRL B-50936), LGP2008 (NRRL B-50937), LGP2009 (NRRL B-50938), LGP2010 (NRRL B-50939), LGP2011 (NRRL B-50940), LGP2012 (NRRL B-50941), LGP2013 (NRRL B-50942), LGP2014 (NRRL B-67339), LGP2015 (NRRL B-67340), LGP2016 (NRRL B-67341), LGP2017 (NRRL B-67741), LGP2018 (NRRL B-67742), LGP2019 (NRRL B-67743), LGP2020 (NRRL B-67892), LGP2021 (NRRL B-68032), LGP2022 (NRRL B-68033), LGP2023 (NRRL B-68034), LGP2029 (NRRL B-68065), LGP2030 (NRRL B-68066), LGP2031 (NRRL B-68067), LGP2033 (NRRL B-68068), LGP2034 (NRRL B-68069), or LGP2167 (NRRL B-67927). In certain embodiments, the percent ANI can be determined as disclosed by Konstantinidis et al., 2006.

In certain embodiments of the methods provided herein, plants, plant seeds, and/or plant parts are treated with both a Methylobacterium strain and at least one additional component. In some embodiments an additional component can be an additional active ingredient, for example, a pesticide or a second biological. In certain embodiments, the pesticide can be an insecticide, a fungicide, an herbicide, a nematicide, or other biocide. The second biological could be a strain that improves yield or controls an insect, pest, fungi, weed, or nematode. In some embodiments, a second biological is an additional Methylobacterium strain. In some embodiments, an additional Methylobacterium strain in the methods and compositions provided herein is selected from the list of Methylobacterium strains in Table 1.

Non-limiting examples of insecticides and nematicides include carbamates, diamides, macrocyclic lactones, neonicotinoids, organophosphates, phenylpyrazoles, pyrethrins, spinosyns, synthetic pyrethroids, tetronic and tetramic acids. In particular embodiments insecticides and nematicides include abamectin, aldicarb, aldoxycarb, bifenthrin, carbofuran, chlorantraniliporle, chlothianidin, cyfluthrin, cyhalothrin, cypermethrin, deltamethrin, dinotefuran, emamectin, ethiprole, fenamiphos, fipronil, flubendiamide, fosthiazate, imidacloprid, ivermectin, lambda-cyhalothrin, milbemectin, nitenpyram, oxamyl, permethrin, tioxazafen, spinetoram, spinosad, spirodichlofen, spirotetramat, tefluthrin, thiacloprid, thiamethoxam, and thiodicarb.

Non-limiting examples of useful fungicides include aromatic hydrocarbons, benzimidazoles, benzthiadiazole, carboxamides, carboxylic acid amides, morpholines, phenylamides, phosphonates, quinone outside inhibitors (e.g. strobilurins), thiazolidines, thiophanates, thiophene carboxamides, and triazoles. Particular examples of fungicides include acibenzolar-S-methyl, azoxystrobin, benalaxyl, bixafen, boscalid, carbendazim, cyproconazole, dimethomorph, epoxiconazole, fluopyram, fluoxastrobin, flutianil, flutolanil, fluxapyroxad, fosetyl-Al, ipconazole, isopyrazam, kresoxim-methyl, mefenoxam, metalaxyl, metconazole, myclobutanil, orysastrobin, penflufen, penthiopyrad, picoxystrobin, propiconazole, prothioconazole, pyraclostrobin, sedaxane, silthiofam, tebuconazole, thifluzamide, thiophanate, tolclofos-methyl, trifloxystrobin, and triticonazole. Non-limiting examples of other biocides include isothiazolinones, for example 1,2 Benzothiazolin-3-one (BIT), 5-Chloro-2-methyl-4-isothiazolin-3-one (CIT), 2-Methyl-4-isothiazolin-3-one (MIT), octylisothiazolinone (OIT), dichlorooctylisothiazolinone (DCOIT), and butylbenzisothiazolinone (BBIT); 2-Bromo-2-nitro-propane-1,3-diol (Bronopol), 5-bromo-5-nitro-1,3-dioxane (Bronidox), Tris(hydroxymethyl)nitromethane, 2,2-Dibromo-3-nitrilopropionamide (DBNPA), and alkyl dimethyl benzyl ammonium chlorides.

Non-limiting examples of herbicides include ACCase inhibitors, acetanilides, AHAS inhibitors, carotenoid biosynthesis inhibitors, EPSPS inhibitors, glutamine synthetase inhibitors, PPO inhibitors, PS II inhibitors, and synthetic auxins. Particular examples of herbicides include acetochlor, clethodim, dicamba, flumioxazin, fomesafen, glyphosate, glufosinate, mesotrione, quizalofop, saflufenacil, sulcotrione, and 2,4-D.

In some embodiments, the composition or method disclosed herein may comprise a Methylobacterium strain and an additional active ingredient selected from the group consisting of clothianidin, ipconazole, imidacloprid, metalaxyl, mefenoxam, tioxazafen, azoxystrobin, thiomethoxam, fluopyram, prothioconazole, pyraclostrobin, and sedaxane.

In some embodiments, the composition or method disclosed herein may comprise an additional active ingredient, which may be a second biological. The second biological could be a biological control agent, other beneficial microorganisms, microbial extracts, plant extracts, yeast extracts, vegetal chitosan, natural products, plant growth activators or plant defense agent. Non-limiting examples of the second biological could include bacteria, fungi, beneficial nematodes, and viruses. In certain embodiments, the second biological can be a Methylobacterium. In certain embodiments, the second biological is a Methylobacterium listed in Table 1. In certain embodiments, the second biological can be a Methylobacterium selected from M. gregans, M. radiotolerans, M. extorquens, M. populi, M salsuginis, M. brachiatum, and M. komagatae.

In certain embodiments, the second biological can be a bacterium of the genus Actinomycetes, Agrobacterium, Arthrobacter, Alcaligenes, Aureobacterium, Azobacter, Azorhizobium, Azospirillum, Azotobacter, Beijerinckia, Bacillus, Brevibacillus, Burkholderia, Chromobacterium, Clostridium, Clavibacter, Comomonas, Corynebacterium, Curtobacterium, Enterobacter, Flavobacterium, Gluconacetobacter, Gluconobacter, Herbaspirillum, Hydrogenophage, Klebsiella, Luteibacter, Lysinibacillus, Mesorhizobium, Methylobacterium, Microbacterium, Ochrobactrum, Paenibacillus, Pantoea, Pasteuria, Phingobacterium, Photorhabdus, Phyllobacterium, Pseudomonas, Rhizobium, Rhodococcus, Bradyrhizobium, Serratia, Sinorhizobium, Sphingomonas, Streptomyces, Stenotrophomonas, Variovorax, Xanthomonas and Xenorhadbus. In particular embodiments the bacteria is selected from the group consisting of Bacillus amyloliquefaciens, Bacillus cereus, Bacillus firmus, Bacillus, lichenformis, Bacillus pumilus, Bacillus sphaericus, Bacillus subtilis, Bacillus thuringiensis, Chromobacterium suttsuga, Pasteuria penetrans, Pasteuria usage, and Pseudomona fluorescens.

In certain embodiments the second biological can be a fungus of the genus Acremonium, Alternaria, Ampelomyces, Aspergillus, Aureobasidium, Beauveria, Botryosphaeria, Cladosporium, Cochliobolus, Colletotrichum, Coniothyrium, Embellisia, Epicoccum, Fusarium, Gigaspora, Gliocladium, Glomus, Laccaria, Metarhisium, Muscodor, Nigrospora, Paecilonyces, Paraglomus, Penicillium, Phoma, Pisolithus, Podospora, Rhizopogon, Scleroderma, Trichoderma, Typhula, Ulocladium, and Verticilium. In particular embodiments, the fungus is Beauveria bassiana, Coniothyrium minitans, Gliocladium vixens, Muscodor albus, Paecilomyces lilacinus, or Trichoderma polysporum.

In certain embodiments, compositions comprise multiple additional biological ingredients, including consortia comprising combinations of any of the above bacterial or fungal genera or species.

In further embodiments the second biological can be a biostimulant, including but not limited to seaweed extract or hummates, plant growth activators or plant defense agents including, but not limited to harpin, Reynoutria sachalinensis, jasmonate, lipochitooligosaccharides, and isoflavones.

In further embodiments, the second biological can include, but are not limited to, various Bacillus sp., Pseudomonas sp., Coniothyrium sp., Pantoea sp., Streptomyces sp., and Trichoderma sp. Microbial biopesticides can be a bacterium, fungus, virus, or protozoan. Particularly useful biopesticidal microorganisms include various Bacillus subtilis, Bacillus thuringiensis, Bacillus pumilis, Pseudomonas syringae, Trichoderma harzianum, Trichoderma virens, and Streptomyces lydicus strains. Other microorganisms that are added can be genetically engineered or wild-type isolates that are available as pure cultures. In certain embodiments, it is anticipated that the second biological can be provided in the composition in the form of a spore.

Plants or harvested plant parts having increased levels of at least one mineral nutrient and/or at least one vitamin in comparison to a control plant or plant part are provided, as are methods for obtaining and using such plants and plant parts. In certain embodiments, the content of at least one mineral nutrient and/or at least one vitamin in the plants or harvested plant part is increased by at least about 1%, or 2% to about 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% per gram dry or wet weight in comparison to the content of the at least one mineral nutrient and/or at least one vitamin in a control plant or plant part. In other embodiments, the content of at least one mineral nutrient and/or at least one vitamin in the plants, plant parts, food ingredients, and feed ingredients is increased by more than 30%, including 35%, 40%, 45%, 50%, or greater than 50% in comparison to the content of the at least one mineral nutrient and/or at least one vitamin in a control plant or plant part. In some embodiments, the content of more than one mineral nutrient and/or more than one vitamin is increased in a plant or harvested plant part, and percent increases can vary for each of the mineral nutrients and/or vitamins, with each increased mineral nutrient and vitamin being increased by at least about 1%, or 2% to about 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% or more per gram dry or wet weight. Controls include plants or plant parts harvested from control plants grown from an untreated control seed or untreated control.

The mineral nutrient and/or vitamin content of plants or harvested parts thereof grown from seeds or seedlings treated with an effective amount of a Methylobacterium strain or strains can be determined by a variety of different techniques or combinations of techniques. Nitrate and nitrite nitrogen content determination methods include Cadmium Reduction and Colorimetric analysis by Flow Injection system (Lachat); AOAC 968.07. Mineral Digestion can be accomplished by Open Vessel Microwave SW846-3051A (AOAC 991-10D(e)). Mineral analysis can be conducted by Inductively Coupled Argon Plasma (ICAP); AOAC 985.01. Mineral nutrients and vitamins content of seeds and various food products can also be determined by standard methods set forth by the AACC, AOAC in Official Methods of Analysis of AOAC INTERNATIONAL, 21st Edition (2019) and in the Codex Alimentarius of International Food Standards set forth by the Food and Agriculture Organization of the United Nations (FAO) or WHO (CXS 234-19991, Adopted in 1999).

### Deposit Information

Samples of the following Methylobacterium sp. strains have been deposited with the AGRICULTURAL RESEARCH SERVICE CULTURE COLLECTION (NRRL) of the National Center for Agricultural Utilization Research, Agricultural Research Service, U.S. Department of Agriculture, 1815 North University Street, Peoria, Illinois 61604 U.S.A. under the terms of the Budapest Treaty on the International Recognition of the Deposit of Microorganisms for the Purposes of Patent Procedure. Methylobacterium sp. NRRL B-50929, NRRL B-50930, NRRL B-50931, NRRL B-50932, NRRL B-50933, NRRL B-50934, NRRL B-50935, NRRL B-50936, NRRL B-50937, NRRL B-50938, NRRL B-50939, NRRL B-50940, NRRL B-50941 and NRRL B-50942 were deposited with NRRL on Mar. 12, 2014. Methylobacterium sp. NRRL B-67339, NRRL B-67340 and, NRRL B-67341 were deposited with NRRL on Nov. 18, 2016. Methylobacterium sp. NRRL B-67741, NRRL B-67742, and NRRL B-67743 were deposited with NRRL on Dec. 20, 2018. Methylobacterium sp. NRRL B-67892 was deposited with NRRL on Nov. 26, 2019. Methylobacterium sp. NRRL B-67925, NRRL B-67926 and NRRL B-67927 were deposited with NRRL on Feb. 21, 2020. Methylobacterium sp. NRRL B-67929 was deposited with NRRL on Mar. 3, 2020. Methylobacterium sp. NRRL B-68032, NRRL B-68033 and NRRL B-68034 were deposited with NRRL on May 20, 2021. Methylobacterium sp. NRRL B-68064, NRRL B-68065, NRRL B-68066, NRRL B-68067, NRRL B-68068, and NRRL B-68069 were deposited with NRRL on Sep. 9, 2021. NRRL-B-68194, NRRL-B-68195, NRRL-B-68196, and NRRL-B-68197 were deposited with NRRL on Aug. 30, 2022. NRRL B-68215, NRRL B-68216, NRRL B-68217, and NRRL B-68218 were deposited with NRRL on Nov. 2, 2022. NRRL B-68236, NRRL B-68237, NRRL B-68238, and NRRL B-68239 were deposited with NRRL on Nov. 23, 2022.

Subject to 37 CFR § 1.808(b), all restrictions imposed by the depositor on the availability to the public of the deposited material will be irrevocably removed upon the granting of any patent from this patent application.

### EXAMPLES

The following examples are given for purely illustrative and non-limiting purposes of the present invention.

### Example 1. Effects of Methylobacterium Strain LGP2009 (NRRL B-50938) Treatment of Spinach on Mineral Nutrient Content of Harvested Leaves

Spinach seeds were treated with Methylobacterium strain LGP2009 at a rate of 10⁶ CFU per seed and grown in soil mix (Fick's garden mix soil) in 15 flats (26 seeds per flat) in a greenhouse in parallel with 15 flats of untreated spinach seeds. Flats were thinned to contain no less than 20 plants. At 28 days after planting (approximately 7 true leaves), 15 or more plants per flat were chosen randomly and shoots were collected by cutting one inch above the soil line. The shoots were incubated in sample bags at 45° C. for 4 days to dry and analyzed for macronutrient and micronutrient content. A single-tailed unequal variances (Welch's) t-test was used to analyze the data to determine whether treatment with LGP2009 resulted in a significant increase in nutrient content. Methylobacterium LGP2009 significantly enhanced foliar content of three nutrients: nitrogen (N), magnesium (Mg), and iron (Fe). Other nutrients elevated over the untreated control sample (UTC) by treatment with LGP2009 were copper, calcium, potassium, and sulfur. Levels of zinc, boron, phosphorus, and manganese were lower in LGP2009 treated plants in comparison to control untreated plants.

Percent differences between the LGP2009 treatment and the UTC treatment for macro- and micronutrients measured in this experiment are shown in Table 2. P-values were estimated using Student's t-test. Results showing a difference at p<0.1 are noted in italics.

TABLE 2

| | | | | % | Contrast |
|---|---|---|---|---|---|
| Nutrient | | LGP2009 | UTC | difference | p-value v. |
| type | Nutrient (units) | value | value | from UTC | UTC |
| Macro- | Nitrogen (%) | 5.454 | 4.855 | +12.3% | 0.023 |
| nutrient | Phosphorus (%) | 0.506 | 0.556 | −8.9% | 0.20 |
| | Potassium (%) | 12.2 | 12.0 | +2.0% | 0.48 |
| | Calcium (%) | 0.92 | 0.88 | +4.6% | 0.41 |
| | Magnesium (%) | 1.27 | 1.09 | +16.2% | 0.045 |
| | Sulfur (%) | 0.463 | 0.456 | +1.5% | 0.59 |
| Micro- | Zinc (ppm) | 129.1 | 151.1 | −14.6% | 0.060 |
| nutrient | Manganese (ppm) | 56 | 57 | −1.8% | 0.69 |
| | Iron (ppm) | 110.1 | 96.9 | +13.6% | 0.086 |
| | Copper (ppm) | 10.9 | 10.2 | +7.0% | 0.18 |
| | Boron (ppm) | 53.7 | 59.4 | −9.7% | 0.033 |

### Example 2. Assay for Methylobacterium Effect on Micronutrient Content and Increased Early Growth in Hydroponic System

The experiment was conducted using a randomized complete block design. An experiment with 3 treatment levels to compare the biomass of plants following seed treatment with 2 Methylobacterium strains and water to a control treated with only water was conducted as follows for testing growth enhancement effects of Methylobacterium isolates. The experiment had an n=10 and was laid out in 10 completely randomized blocks. Each experimental unit consisted of 24 individual plants grown on a quarter (3×8 cubes) sheet of horticube and bulked for biomass.

Ten horticube sheets (104 cell Oasis HorticubeXL™, single dibble; Smithers-Oasis North America, Kent, OH, USA) were each divided into four 3×8 cube pieces, and 30 pieces were placed into their own clean 1020 mesh tray. The horticube pieces were completely saturated with UV filtered R.O. water, and one seed (lettuce or spinach) was placed in each dibble (pre-formed seed hole) of the horticubes. Seeds were inoculated by applying 10⁶ CFU of a Methylobacterium strain to be tested directly to each seed.

Seeds were allowed to grow undisturbed at 23-25° C. and 14 hour days. Plants were broadcast watered and fertilized (15-16-17) on Mondays, Wednesdays and Fridays. Plants were watered with UV filtered RO water on all other days. Fourteen days after planting (approximately 2 true leaf stage), the shoot portion of each plant was harvested by cutting directly below the cotyledon and all the shoots from the same tray were bulked together. The shoots were allowed to dry in an oven at 45° C. for at least 3 days and the bulked shoots from each sheet/tray weighed to identify Methylobacterium strains that increase shoot biomass in lettuce or spinach following seed treatment. Shoots may be from the same samples as measured to determine biomass or from a separate experiment conducted as described in Example 1. 00911 Results of analysis of the effect of treatment with various Methylobacterium strains on enhanced early growth of 2 true leaf stage lettuce and spinach plants as described above are provided in Tables 3 and 4 below. Lettuce results in Table 3 are from biomass data only. Data are combined results from at least 3 independent repetitions of an experiment with a given isolate. Contrast p-values were taken from Student's t-test post hoc to a linear mixed model. The lettuce results in Table 3 show that using LGP2002, LGP2001, LGP210, LGP2012, LGP2000, LGP2009, LGP2006, LGP2011, LGP2007, LGP2004, LGP2025, LGP2026, LGP2021, LGP2020, LGP2017, LGP2028, LGP2029, LGP2030, LGP2019, LGP2031, LGP2016, LGP2033, LGP2034, LGP2022, LGP2023, and a combination of LGP2002 and LGP2015 results in a positive percent growth enhancement over control.

TABLE 3
Lettuce Growth Measurement

| | Percent growth enhancement | Contrast p-value |
|---|---|---|
| Treatment | over Control | vs. Control |
| LGP2002 | +2.9% | 0.24 |
| LGP2001 | +8.4% | 0.035 |
| LGP2010 | +9.7% | 0.0038 |
| LGP2012 | +4.3% | 0.0025 |
| LGP2000 | +7.0% | 0.035 |
| LGP2009 | +9.6% | 0.017 |
| LGP2006 | +5.3% | 0.44 |
| LGP2011 | +2.7% | 0.24 |
| LGP2007 | +9.5% | 0.0043 |
| LGP2004 | +1.4% | 0.56 |
| LGP2024 | −10.5% | 0.14 |
| LGP2025 | +4.1% | 0.53 |
| LGP2026 | +8.2% | 0.23 |
| LGP2021 | +7.8% | 0.0007 |
| LGP2027 | −3.0% | 0.66 |
| LGP2020 | +1.8% | 0.26 |
| LGP2017 | +1.2% | 0.14 |
| LGP2028 | +1.3% | 0.24 |
| LGP2029 | +5.3% | 0.0038 |
| LGP2030 | +2.8% | 0.06 |
| LGP2019 | +2.7% | 0.22 |
| LGP2031 | +0.3% | 0.64 |
| LGP2032 | −7.6% | 0.27 |
| LGP2016 | +1.7% | 0.89 |
| LGP2033 | +2.0% | 0.13 |
| LGP2034 | +4.8% | 0.011 |
| LGP2022 | +10.9% | 0.011 |
| LGP2023 | +4.6% | 0.047 |
| LGP2002 + LGP2015 | +5.3% | 0.0043 |

Spinach results in Table 4 are based on image data as a proxy for aboveground biomass. Data are combined results from 2 independent repetitions of experiment. Contrast p-values were taken from Student's t-test post hoc to a linear mixed model. The spinach results in Table 4 show that using LGP2001, LGP2010, LGP2009, LGP2021, LGP2022, LGP2023, and a combination of LGP2002 and LGP2015 results in a positive percent growth enhancement over control.

TABLE 4
Spinach Growth Measurement

| | Percent growth | Contrast p-value |
|---|---|---|
| Treatment | enhancement over Control | vs. Control |
| LGP2001 | +2.7% | 0.33 |
| LGP2010 | +2.0% | 0.48 |
| LGP2009 | +0.7% | 0.81 |
| LGP2021 | +0.8% | 0.78 |
| LGP2022 | +4.0% | 0.15 |
| LGP2023 | +1.9% | 0.49 |
| LGP2002 + | +1.4% | 0.62 |
| LGP2015 | | |

### Example 3. Detection or Identification of Methylobacterium Strains, Variants and Derivatives

Assays are disclosed for detection or identification of specific Methylobacterium strains and closely related derivatives. Genomic DNA fragments unique to a Methylobacterium strain were identified and qPCR Locked Nucleic Acid (LNA) based assays were developed.

Genomic DNA sequences of Methylobacterium strains were compared by BLAST analysis of approximately 300 bp fragments using a sliding window of from 1-25 nucleotides to whole genome sequences of over 1000 public and proprietary Methylobacterium isolates. Genomic DNA fragments were identified that have weak BLAST alignments, indicative of approximately 6095 identity over the entire fragment, to corresponding fragments of a Methylobacterium of interest. Fragments from the LGP2015 genome corresponding to the identified weak alignment regions were selected for assay development and are provided as SEQ ID NOS: 1-3.

TABLE 5
Unique Fragment Sequences of LGP2015

| | SEQ | |
|---|---|---|
| | ID | |
| Fragment | NO | Sequence |
| ref1_ | 1 | ACGGTCACCCCACGGACTGGGCGAGTACCTCACCGG |
| 135566 | | TGTTCTATCATAACGCCGAGTTAGTTTTCGACCGTCC |
| | | CTTATGCGATGTACCACCGGTGTCGGCAGCCGATTT |
| | | CGTCCCACCGGGAGCTGGCGTTCCGGTTCAGACCAC |
| | | CATCATCGGTCACGATGTCTGGATTGGACACGGGGC |
| | | CTTCATCTCCCCCGGCGTGACTATAGGAAACGGCGC |
| | | GATCGTCGGGGCCCAGGCGGTCGTCACAAGAGATGT |
| | | CCCACCCTATGCGGTAGTTGCTGGCGTCCCCGCGAC |
| | | CGTACGACGAT |
| ref1_ | 2 | CCAATAAAAGCGTTGGCCGCCTGGGCAACCCGATCC |
| 135772 | | GAGCCTAAGACTCAAAGCGCAAGCGAACACTTGGTA |
| | | GAGACAGCCCGCCGACTACGGCGTTCCAGCACTCTC |
| | | CGGCTTTGATCGGATAGGCATTGGTCAAGGTGCCGG |
| | | TGGTGATGACCTCGCCCGCCGCAAGCGGCGAATTAC |
| | | TCGGATCAGCGGCCAGCACCTCGACCAAGTGTCGGA |
| | | GCGCGACCAAAGGGCCACGTTCGAGGACGTTTGAGG |
| | | CGCGACCAGTCTCGATAGTCTCATCGTCGCGGCGAA |
| | | GCTGCACCTCGA |
| ref1_ | 3 | CGATGGCACCGACCTGCCATGCCTCTGCCGTCCGCG |
| 169470 | | CCAGAATGGTAAAGAGGACGAAGGGGGTAAGGATC |
| | | GTCGCTGCAGTGTTGAGCAGCGACCAGAGAAGGGG |
| | | GCCGAACATCGGCATCAAACCTCGATTGCCACTCGG |
| | | ACGCGAAGCGCGTCTTGAAGGAGGGATGGAAGCGA |
| | | AACGGCCGCAGAGTAACCGCCGACGAAAGATTGCA |
| | | CCCCTCATCGAGCAGGATCGGAGGTGAAGGCAAGC |
| | | GTGGGTTATTGGTAAGTGCAAAAAATATAATGGTAG |
| | | CGTCAGATCTAGCGTTC |

Regions in SEQ ID NOS: 1-3 where corresponding regions in other Methylobacterium strains were identified as having one or more nucleotide mismatches from the LGP2015 sequence were selected, and qPCR primers, designed using Primer3 software (Untergasser et al. (2012), Koressaar et al. (2007)) to flank the mismatch regions, have a melting temperature (Tm) in the range of 55-60 degrees and generate a PCR DNA fragment of approximately 100 bp. The probe sequence was designed with a 5′ FAM reporter dye and a 3′ Iowa Black FQ quencher and contains one to six LNA bases (Integrated DNA Technologies, Coralville, Iowa). At least 1 of the LNA bases was in the position of a mismatch, while the other LNA bases were used to raise the Tm. The Tm of the probe sequence was targeted to be 10 degrees above the Tm of the primers.

Primer and probe sequences for detection of specific detection of LGP2015 are provided as SEQ TD NOS: 4-12 in Table 6. Each of the probes contains a 5′ FAM reporter dye and a 3′ Iowa Black FQ quencher.

TABLE 6
Primer and Probe Sequences for
Specific Detection of LGP2015

| | SEQ | |
|---|---|---|
| | ID | |
| Primer/Probe | NO | Sequence* |
| LGP2015_ref1_135566_ | 4 | CCTCA |
| forward | | CCGGT |
| | | GTTCT |
| | | ATCAT |
| | | AAC |
| LGP2015_ref1_135566_ | 5 | CCGAT |
| reverse | | GATGG |
| | | TGGTC |
| | | TGAAC |
| LGP2015_ref1_135566_ | 6 | CGTCC |
| probe | | CTTAT |
| | | GCGAT |
| | | GTACC |
| | | A |
| LGP2015_ref1_135772_ | 7 | GATCC |
| forward | | GAGCC |
| | | TAAGA |
| | | CTCAA |
| | | AG |
| LGP2015_ref1_135772_ | 8 | GACCA |
| reverse | | ATGCC |
| | | TATCC |
| | | GATCA |
| | | A |
| LGP2015_ref1_135772_ | 9 | AACAC |
| probe | | TTGGT |
| | | AGAGA |
| | | CAGCC |
| LGP2015_ref1_169470_ | 10 | AAGGA |
| forward | | GGGAT |
| | | GGAAG |
| | | CGAAA |
| | | C |
| LGP2015_ref1_169470_ | 11 | ATAAC |
| reverse | | CCACG |
| | | CTTGC |
| | | CTTC |
| LGP2015_ref1_169470_ | 12 | CGCAG |
| probe | | AGTAA |
| | | CCGCC |
| | | GACGA |
| | | A |

*Bold and underlined letters represent the position of an LNA base.

Use of Primer/Probe Sets on Isolated DNA to Detect LGP2015 and Distinguish from Related Methylobacterium Isolates

Each 10 ul qPCR reaction contained 5 ul of Quantabio PerfeCTa qPCR ToughMix 2× Mastermix, Low ROX from VWR, 0.5 ul of 10 uM forward primer, 0.5 ul of 10 uM reverse primer, 1 ul of 2.5 uM probe, 1 ul nuclease free water, and 2 ul of DNA template. Approximately 1 ng of DNA template was used per reaction. The reaction was conducted in a ThermoFisher QuantStudio™ 6 Flex Real-Time PCR System with the following program: 95° C. for 3 min, then 40 cycles of 95° C. for 15 sec, and 60° C. for 1 min. The analysis software on the PCR instrument calculated a threshold and Ct value for each sample. Each sample was run in triplicate on the same qPCR plate. A positive result was indicated where the delta Ct between positive and negative controls was at least 5.

Use of the three primer/probe sets to distinguish LGP2015 from closely related isolates by analysis of isolated DNA is shown in Table 7 below. The similarity score shown for the related isolates takes into account both the average nucleotide identity and the alignment fraction between the isolates and LGP2015. One of the tested strains, LGP2035, was used as an additional positive control. LGP2035 is a clonal isolate of LGP2015 which was obtained from a culture of LGP2015, which was confirmed by full genome sequencing as identical to LGP2015, and which scored positive in all three reactions. The similarity score of greater than 1.000 for this strain was likely the result of a slightly different assembly of the genome for this isolate compared to LGP2015. The delta Ct of approximately 15 or more between the LGP2015 and LGP2035 isolates and the water only control is consistent with the sequence confirmation of the identity of these isolates. Analysis of other isolates that are less closely related to LGP2015 resulted in delta Ct values similar to those for the water only control.

TABLE 7

| | Similarity | | | |
|---|---|---|---|---|
| | score to | Average Ct Value | | |
| LGP# | LGP2015 | Ref1_135566 | Ref1_135772 | Ref1_169470 |
| LGP2035 | 1.005 | 21.08 | 21.31 | 20.35 |
| LGP2015 | 1 | 21.97 | 22.62 | 22.08 |
| LGP2036 | 0.181 | No Ct | 37.85 | >37.91 |
| LGP2037 | 0.87 | >36.8 | >38.31 | No Ct |
| LGP2038 | 0.88 | >38.36 | >38.36 | >38.44 |
| LGP2039 | 0.894 | No Ct | >37.47 | >38.13 |
| LGP2031 | 0.852 | 37.81 | No Ct | 37.97 |
| LGP2040 | 0.862 | 37.94 | 38.37 | >38.35 |
| LGP2034 | 0.807 | 38.44 | No Ct | No Ct |
| LGP2041 | 0.894 | 38.77 | No Ct | >37.91 |
| LGP2042 | 0.872 | 37.64 | 37.20 | 37.96 |
| H₂O only | | >38.14 | >35.92 | >37.12 |

### Use of Primer/Probes for Detection of LGP2015 on Treated Plant Materials.

For detection of LGP2015 foliar spray treatment on corn: Untreated corn seeds were planted in field soil in the growth chamber and watered with non-fertilized R.O. water. After plants germinated and grew for approximately 3 weeks, they were transferred to the greenhouse. At V5 stage, plants were divided into 3 groups for treatment: foliar spray of LGP2015, mock foliar spray, and untreated. Plants receiving the foliar spray of LGP2015 were treated with 10× glycerol stock at the rate of 71.4 ul per plant using Solo sprayers. This converts to the rate of 10 L/acre in the field. Mock treated plants were sprayed with 71.4 ul water/plant. Untreated plants received no foliar spray treatment. Leaves were harvested two weeks after foliar spray treatment into sterile tubes and DNA from bacteria on the harvested leaves was isolated as described above. Each experiment was grown at least 2 times. As shown in Table 8, LGP2015 was detected on leaves harvested from corn plants treated by a foliar spray application of the Methylobacterium strains using all 3 primer probe sets, as demonstrated by delta Ct values of approximately 10 between the sample and the negative controls.

TABLE 8

| | Average Ct Value | | |
|---|---|---|---|
| Treatment | Ref1_135566 | Ref1_135772 | Ref1_169470 |
| Control (no | 32.43 | 32.10 | 31.55 |
| application) | | | |
| Control (mock | 35.54 | 35.34 | 34.80 |
| application) | | | |
| LGP2015 (10 L/acre | 23.36 | 22.88 | 22.66 |
| equivalent) | | | |

The above results demonstrate the use of genome specific primers and probes to detect Methylobacterium strain LGP2015 on various plant tissues following treatment with the strains and provide methods to distinguish LGP2015 from closely related isolates. Similar methods were developed for additional Methylobacterium strains LGP2002, LGP2019, LGP2018, and LGP2017 using target sequence fragments and primer/probe pairs as shown in the Tables below.

TABLE 9
Target Fragment Sequences of LGP2002

| | SEQ | |
|---|---|---|
| | ID | |
| Fragment | NO | Sequence |
| ref4_930 | 13 | GCAAAACGACCTAATAGTTCTACAG |
| | | CGGCATGCGCCAAGTCAGCGCGGTG |
| | | AACAGTATACCTGGGAGCAACTTGT |
| | | CCTCCGAAACCCACATAAAACAAAT |
| | | TACTCCTGGCAGTGCCCAGTCCATC |
| | | AAAATCGAATACAATATTTCTCGAG |
| | | GAGGCATCTGTAATAGCCTGCCAAA |
| | | GCAACAAAGCTATGGCGCCGTTATG |
| | | ACTTTCATTGCTTCTGGTAGACATA |
| | | AAATAATATGCCGATTTGTGATCCC |
| | | AAATGTAGAATATTGCCGCATCAAT |
| | | TGCGCCAAGTTTATTTCGGATCGAT |
| ref1_142021 | 14 | GGCGCCAACGGTATGATCGCATGAT |
| | | TTTCCTGCGGCATAGCTTGCGGGAA |
| | | TGGCGTATTTGGCGCTCTCCTCAGG |
| | | AATTTCTAAGGGCATACGCAGGAAC |
| | | TCTACAGCACTTTTACTGGTATTTT |
| | | GTAGTGACAGCGGAGGAGGCTGGTG |
| | | CTCAAGGTAATCGTGATGAAGTGAT |
| | | CCGGGCCATTCGGGGCGCGTTTCTA |
| | | GTCTTTCCAATCCGCGCCCTGTACC |
| | | ACGTATTACGCCGGACCGGTCTGCG |
| | | CCGCGCCGCCCTCTTGACCGCCCTA |
| | | AATGTCTAAGAGCGTCTAACAAAGC |
| ref1_142636 | 15 | GACGATATCGCTCATCTTCACTGCA |
| | | TTGAAGCTGGTGCCGTACTGCATAG |
| | | GGATGAAAAAGTGATGCGGATAGAC |
| | | GGCTGACGGGAAAGCGCCTGGTCGA |
| | | TCGAAGACTTTGCTGACGAGGTTGT |
| | | GGTAGCCCCGGATATAGGCATCGAA |
| | | GGCCGGGACGTTGATCCCATCCTTT |
| | | GCCTTATCTTGACTGGCGTCGTCGC |
| | | GTGCCGTCAGAACGGGCACGTCGCA |
| | | GGTCATCGAGGCCAGCACCTTGCGG |
| | | AACACCTGCGTTCCGCCGTTGGGAT |
| | | TATCGACGGCGAACGCGGTGGCCGC |

TABLE 10
Primer and Probe Sequences for
Specific Detection of LGP2002

| | SEQ | |
|---|---|---|
| | ID | |
| Primer/Probe | NO | Sequence* |
| LGP2002_ref4_930_forward | 16 | GTCCTCCGAA |
| | | ACCCACATAA |
| | | A |
| LGP2002_ref4_930_reverse | 17 | CTACCAGAAG |
| | | CAATGAAAGT |
| | | CAT |
| LGP2002_ref4_930_probe | 18 | TCTGTAATAG |
| | | CCTGCCAAAG |
| | | CA |
| LGP2002_ref1_142021_forward | 19 | GGCTGGTGCT |
| | | CAAGGTAAT |
| LGP2002_ref1_142021_reverse | 20 | ACATTTAGGG |
| | | CGGTCAAGAG |
| LGP2002_ref1_142021_probe | 21 | ATGAAGTGAT |
| | | CCGGGCCAT |
| LGP2002_ref1_142636_forward | 22 | CCGTACTGCA |
| | | TAGGGATGAA |
| | | A |
| LGP2002_ref1_142636_reverse | 23 | TAAGGCAAAG |
| | | GATGGGATCA |
| | | A |
| LGP2002_ref1_142636_probe | 24 | TTGCTGACGA |
| | | GGTTGTGGTA |
| | | G |

*Bold and underlined letters represent the position of an LNA base.

TABLE 11
Target Fragment Sequences of LGP2019

| | SEQ | |
|---|---|---|
| | ID | |
| Fragment | NO | Sequence |
| ref1_458355 | 25 | CAACTATGTAGACCCGACGG |
| | | TGCGATTTCACTTCGCAAAG |
| | | CCGCAGGGCAGCACCCTTGC |
| | | GCTCAATGTTGACGCCAGCG |
| | | TGATCTATACTATTACCGTC |
| | | ACGCACACGCAGGGCGGCGT |
| | | ACAGATTCATCGCGAGAGTA |
| | | AGAACCACCATCAGACCATC |
| | | ACGCGCAGCGACCTGAGCAA |
| | | GCAGTTCGGCGTTGGTGTGG |
| | | CCGACCAGCTGACGCGCGAT |
| | | CAGGTCATGAAGGTGATCGA |
| | | GTCGGCATTTCGCGACGCTA |
| | | CCCGCTAAGATCGGCGCCCA |
| | | CGAAACGCTACGAGACTAGG |
| ref1_459688 | 26 | AGCCGGCATCTTGTTCAAGG |
| | | CGCTCACCTCGACGCCGACG |
| | | CTGTAGGCGACTTGAGAGGG |
| | | CGTCTCATATGAACGAAGCA |
| | | TCTTCGCGTAGAGAACCTTC |
| | | TTGTTCTCCTGCGTGATGTT |
| | | CGCTTTGCAGACGTTGACTG |
| | | CCGCCATGAACGCCGAAGCC |
| | | TTGCGCGCTTCATCGTAATC |
| | | GCCTGCGAAGGCGGGTAGTG |
| | | AAAAGCTTAGTGCAATGGCA |
| | | AACACAGCCGCCGAACGTCG |
| | | CATGGTATCCGTCCCCGATT |
| | | GACGGCAGTGCCGCCATATC |
| | | TCGGCTTTAGCAGAGCTGAT |
| ref1_3158527 | 27 | AACCTGCGCCGGCCGAGGTT |
| | | TCGCGAGCCGTCGCCACGGG |
| | | CAACGCCTCGCCCGCGATGT |
| | | GCAAAAAAGTCCCCGGCACT |
| | | TCGCGCCGTCGTCCGATCCA |
| | | CGACCGCGAATTTCTCAACG |
| | | AGTACAAGGTGCTTATGGGA |
| | | GATCCGAGCGTCCGTCCCGG |
| | | AGCCCGAGACCGCGCGGCCC |
| | | GAGTAATAGGCGAAAAAGAC |
| | | TCCTACTCCTCGGGCTTCTC |
| | | GGGCCCCCTCAGCAACATCT |
| | | ACGCTTGCCGCCCATCACCC |
| | | TGGCGGGAGATCAGCGACGA |
| | | GACACAGGCCCACTTCGCCC |

TABLE 12
Primer and Probe Sequences for
Specific Detection of LGP2019

| | SEQ | |
|---|---|---|
| | ID | |
| Primer/Probe | NO | Sequence* |
| LGP2019_ref1_458355_forward | 28 | TTGACGCCAGCGTGATCTATAC |
| LGP2019_ref1_458355_reverse | 29 | GTGATGGTCTGATGGTGGTTCT |
| LGP2019_ref1_458355_probe | 30 | TATTACCGTCACGCACACG |
| LGP2019_ref1_459688_forward | 31 | CTTCGCGTAGAGAACCTTCTTGTT |
| LGP2019_ref1_459688_reverse | 32 | CTTCGCAGGCGATTACGATGAA |
| LGP2019_ref1_459688_probe | 33 | CGTGATGTTCGCTTTGCAGA |
| LGP2019_ref1_3158527_forward | 34 | CCGCGAATTTCTCAACGAGTACA |
| LGP2019_ref1_3158527_reverse | 35 | GCCCGAGGAGTAGGAGTCTTT |
| LGP2019_ref1_3158527_probe | 36 | AGGTGCTTATGGGAGATCCG |

*Bold and underlined letters represent the position of an LNA base.

Use of the primer/probe sets to distinguish LGP2019 from closely related isolates by analysis of isolated DNA is shown in Table 13 below. The similarity score shown for the related isolates took into account both the average nucleotide identity and the alignment fraction between the isolates and LGP2019. Two of the tested strains, LGP2043 and LGP2014, were used as additional positive controls since a similarity score of 1.00 indicates they are nearly identical to LGP2019. Consistently low Ct values from qPCR using LGP2019 as the DNA template and no detection in the water only control is consistent with the sequence confirmation of the identity of these isolates. Analysis of other isolates that are less closely related to LGP2019 resulted in no detection similar to those for the water only control.

TABLE 13

| | Similarity to | Average Ct Value | | |
|---|---|---|---|---|
| LGP# | LGP2019 | ref1_459688 | ref1_3158527 | ref1_458355 |
| LGP2019 | 1.00 | 22.39 | 24.09 | 23.10 |
| LGP2043 | 1.00 | 22.49 | 24.04 | 22.96 |
| LGP2014 | 1.00 | 22.49 | 23.86 | 22.90 |
| Strain A | 0.95 | UDT | UDT | UDT |
| Strain B | 0.94 | UDT | UDT | UDT |
| Strain C | 0.93 | UDT | UDT | UDT |
| Strain D | 0.93 | UDT | UDT | UDT |
| water only | — | UDT | UDT | UDT |
| (neg control) | | | | |

TABLE 14
Target Fragment Sequences of LGP2017

| | SEQ | |
|---|---|---|
| | ID | |
| Fragment | NO | Sequence |
| ref1_ | 37 | AGTCATTGATCAAGCAACCCCTATT |
| 1185955 | | GAGTTGGATATCGAAGGATCAAGGT |
| | | CGCGTCAATAGATGCATCTATCAGG |
| | | CCAAATGTCGCTTTTCAAGAATGGC |
| | | TCTTTCGAAGCTATCTTTATAATCG |
| | | CTCGCCATTCTCTCATTACCAAAAT |
| | | CGACCTTAACTAGCTCGACATTGAT |
| | | GCGAGCAGCTCCGGCAAACGAGGAG |
| | | AGATTGACCTTAAAGGAATTGAACG |
| | | CCTCAAGCAATTCAGACACATTACC |
| | | AGGAGTGCTATAGCAACAACCAGAC |
| | | CCATATCGGTCAATAACCTCTTTTA |
| ref1_ | 38 | CGCAAAACGATTTATCACTGCCATC |
| 3282585 | | TTGTTGTTTGATAACCCTTTTTTAC |
| | | CAGACGTTATGCTGGGCGAGAAAGA |
| | | GGACTAGCAGATCGGAGCGGTATCG |
| | | CGATTTTTCGGTAGTTCGCGCCTAC |
| | | AACAGGATAAGATCCGATAGTGAAG |
| | | CAACATGGCTGTTTTTTGATTTGTA |
| | | AGTCAGCAACTTAAGCAGCCAGCCT |
| | | ATCTGCCGTCGCAGACGCTTGAGGC |
| | | ATCGGGCAGCATCTTAGAAAAGGTG |
| | | GCAGTAATTGCCACAGCGGAACGTA |
| | | GCGGCACGGATAAGCACGCAGGGTC |
| ref1_ | 39 | CCCATCTGGACCCAATATCCCCTTC |
| 4194637 | | ATCGACAATTCCCGAGTAAGTGTGG |
| | | GTTCGAGGATTTCGCGAAACAGCCT |
| | | TGTTCGTTCCTCCGGCCTTAAAATT |
| | | GGCGTGCCGTCGGGAGATCGATAGG |
| | | CATCCCTTACCTGCCTTTCGACCGC |
| | | CGGCACACGCGCGCCGGTCGTCGTG |
| | | TTCACGGCCACGGAATGGACGAAGG |
| | | TGCGCCGCTCATTTCGCTCGTTTGC |
| | | CGTCTCCACCATCCAGGAGGCCAGC |
| | | AGGACGGTTTCGTCTCGACCGCCGG |
| | | TCACACACACCGCAAGGGACTCAGG |

TABLE 15
Primer and Probe Sequences for
Specific Detection of LGP2017

| | SEQ | |
|---|---|---|
| | ID | |
| Primer/Probe | NO | Sequence* |
| LGP2017_ref1_1185955_ | 40 | TCGCTCGCCA |
| forward | | TTCTCTCATT |
| | | AC |
| LGP2017_ref1_1185955_ | 41 | AGGTCAATCT |
| reverse | | CTCCTCGTTT |
| | | GC |
| LGP2017_ref1_1185955_ | 42 | TCGACATTGA |
| probe | | TGCGAGCA |
| LGP2017_ref1_3282585_ | 43 | TTCGCGCCTA |
| forward | | CAACAGGATA |
| | | AG |
| LGP2017_ref1_3282585_ | 44 | CAGATAGGCT |
| reverse | | GGCTGCTTAA |
| | | GTT |
| LGP2017_ref1_3282585_ | 45 | TCCGATAGTG |
| probe | | AAGCAACA |
| LGP2017_ref1_4194637_ | 46 | GAGTAAGTGT |
| forward | | GGGTTCGAGG |
| | | ATTT |
| LGP2017_ref1_4194637_ | 47 | AGGTAAGGGA |
| reverse | | TGCCTATCGA |
| | | TCT |
| LGP2017_ref1_4194637_ | 48 | CGGAGGAACG |
| probe | | AACAAGGC |

*Bold and underlined letters represent the position of an LNA base.

TABLE 16
Target Fragment Sequences of LGP2018

| | SEQ | |
|---|---|---|
| | ID | |
| Fragment | NO | Sequence |
| LGP2018_ref1_ | 49 | ACCTGCTAAAATCACGTCCT |
| 4871392 | | CTCAGATTGAAAAATCATTG |
| | | AAGAAACGTGTCGAACGATT |
| | | GCCGGGGATTATGACGTTAG |
| | | ATCAATTGAAAAATACAAGC |
| | | TTTGAAATTGAGTTACAGCC |
| | | AAAAGATGCCCCGGATCCGG |
| | | ACCCATCAGACTTCGGTGGC |
| | | TAGTTCGAGCCAAACTCGAA |
| | | CGTCGCCATGGCGCGCAAGT |
| | | CGCAATACCATTTCACAGCG |
| | | CAGCGGTTATTTCGTTGTAC |
| | | ACTGTAGCAATGCGTCGGCT |
| | | TGCGCGCTTCCGCTGGCGAT |
| | | CAAAGGTCCGCCGATTTACG |
| LGP2018_ref1_ | 50 | TCCCGAACATACAATGGAGG |
| 1266930 | | AAGCGTGTGGTAGGCCAATT |
| | | TGTAACGAAATATGGCATCG |
| | | GTCACGGCTCTCTCAATAAA |
| | | TTCGATCTCAAGTCTTCTGA |
| | | ACGAGCATGCCTCATCCTTA |
| | | TCCTGAGCGAACGCCTGCCA |
| | | GTTTGCAGTCATTCCAACAT |
| | | ACATAGCCAAAAAGGCGAGG |
| | | TAGACCTTCATACGGGCACC |
| | | TCAATCGTCCCCATTCGTTC |
| | | AAGCTCCTTCAAGATAACAG |
| | | CCGCACCACATTGCTGAGAT |
| | | CGAAGATTCGGATCAAATAT |
| | | TCCATCAAATTTATACTTTC |
| LGP2018_ref1_ | 51 | GCATCCTTTGCGCTCGCAGG |
| 17614 | | CCTAAGGTCAAGCCCGGTTA |
| | | CTTCGTTTGGTAGAACGAGG |
| | | TAGACGATGCCTAGTCTTAA |
| | | GGTGGCCCATGTTAACCAAC |
| | | AGGGCCAGAACATGATTATA |
| | | GTTCCGTTAGATGCCAACTT |
| | | CGGTTACAAAACCGATGGTG |
| | | AGCAGTCCGACATCATGTTC |
| | | GAAATACAGGACGCGGCGCG |
| | | GTCCGCCGGTCTTGCGGGTG |
| | | CCGTAGTAGCGTTCTGGCAG |
| | | TCAGGTGGACAAACCCGTTT |
| | | CCGGGGCCCGGCTCCGTGGC |
| | | ACCCATTCCTTCGCAGCCTC |

TABLE 17
Primer and Probe Sequences for
Specific Detection of LGP2018

| | SEQ | |
|---|---|---|
| | ID | |
| Primer/Probe | NO | Sequence* |
| LGP2018_ref1_4871392_ | 52 | GCGCAAGTCG |
| forward | | CAATACCATT |
| | | TC |
| LGP2018_ref1_4871392_ | 53 | CGTAAATCGG |
| reverse | | CGGACCTTTG |
| | | A |
| LGP2018_ref1_4871392_ | 54 | CGCAGCGGTT |
| probe | | ATTTCGTTG |
| LGP2018_ref1_1266930_ | 55 | ACGAGCATGC |
| forward | | CTCATCCTTA |
| | | TC |
| LGP2018_ref1_1266930_ | 56 | CGATTGAGGT |
| reverse | | GCCCGTATGA |
| | | A |
| LGP2018_ref1_1266930_ | 57 | TGCCAGTTTG |
| probe | | CAGTCATTCC |
| LGP2018_ref1_17614_ | 58 | CCCGGTTACT |
| forward | | TCGTTTGGTA |
| | | GAA |
| LGP2018_ref1_17614_ | 59 | CGAAGTTGGC |
| reverse | | ATCTAACGGA |
| | | ACTA |
| LGP2018_ref1_17614_ | 60 | TGGCCCATGT |
| probe | | TAACCAACAG |

*Bold and underlined letters represent the position of an LNA base.

### Use of Primer/Probes for Detection of LGP2019 on Treated Plant Materials

Detection of LGP2019 from In-Furrow Treated Corn Roots

At planting, corn seeds in soil were drenched with LGP2019 and control strains from frozen glycerol stock to simulate in-furrow treatment. To obtain a final concentration of 10⁷ CFU/seed, 100 ul of each strain at 10⁸ CFU/ml was inoculated onto each seed placed in the dibble holes in soil. A 1/10 dilution series was made for lower concentration targets. For control treatment, 100 ul Milli-Q water was applied to each corn seed placed in the dibble holes in soil. Pots containing treated seeds were placed in a growth chamber for approximately two weeks and watered with unfertilized RO water every 1-2 days to keep soil moist. After 2 weeks of growth, roots of about 9 plants per replicate sample were harvested into sterile tubes. Each treatment had at least 2 replicate samples in each experiment, and each experiment was conducted at least 3 times.

DNA from bacteria on the harvested corn roots was isolated as follows. Individual roots were submerged in 20 mL of phosphate-buffered saline (PBS) (137 mM NaCl, 10 mM Phosphate, 2.7 mM KCl, and a pH of 7.4) in 50 mL conical tubes. Tubes were vortexed for 10 minutes, and then sonicated for 10 minutes. Root tissue was removed, and the remaining supernatant from multiple roots of the same sample were combined and centrifuged at 7500×g for 10 minutes. This process was repeated until there is one tube for each sample. The moist soil pellet was vortexed until it evenly coats the tube wall. Tubes were placed into a laminar flow hood with caps removed and open ends of the tubes facing the air blowers. Once dry, samples were stored at room temperature. 250 mg dried soil was used as input for DNA extraction using Qiagen DNeasy PowerSoil HTP 96 kit (Cat #12955-4) using manufacturer protocols.

Primers and probes for LGP2019 disclosed in Table 12 above were used in qPCR reactions to detect the presence of LGP2019 specific fragments provided in Table 11. Each 10 ul qPCR reaction contained 5 ul of Quantabio PerfeCTa qPCR ToughMix 2× Mastermix, Low ROX from VWR, 0.5 ul of 10 uM forward primer, 0.5 ul of 10 uM reverse primer, 1 ul of 2.5 uM probe, 1 ul nuclease free water, and 2 ul of DNA template. Approximately 1 ng of DNA template was used per reaction. The reaction was conducted in a ThermoFisher QuantStudio™ 6 Flex Real-Time PCR System with the following program: 95° C. for 3 min, then 40 cycles of 95° C. for 15 sec, and 60° C. for 1 min. The analysis software on the PCR instrument calculated a threshold and Ct value for each sample. Each sample was run in triplicate on the same qPCR plate. A positive result was indicated where the delta Ct between positive and negative controls is at least 5.

### Use of Primer/Probes for Detection of Variants of Additional Table 1 Methylobacterium Isolates

Variants of Methylobacterium isolates listed in Table 1 were identified by the presence of DNA fragments as described above. Unique fragments for use in such methods are provided in Table 18.

TABLE 18

| | | SEQ | |
|---|---|---|---|
| | | ID | |
| Strain | Fragment | NO | Sequence |
| LGP2001 | ref3 | 61 | GCCCTTCTGTCAGGCGATAT |
| | 25009 | | TGTATAATGGCGTTGCCCCA |
| | | | ATAGAAGCAGCCATTCGTGC |
| | | | GAGGGCAGCAGCGACGCTAG |
| | | | GTCGAAAGAGCATCCTAATC |
| | | | TCGATCAAGATGCGACTGAG |
| | | | ATTTCTGATGAAAATATCTA |
| | | | GACACAAGCAAAGCTGGTGA |
| | | | AATTACAACGATCATGGCGA |
| | | | CAATTGCGGCCAATTCGGCC |
| | | | GGAACTTGAAGGAACATAAA |
| | | | AATGAATATTACAAATATAC |
| | | | CGCAAAGCATGTAGAGTTGC |
| | | | TACACCAAGGGTCGGGACGT |
| | | | CCAAAAAAACTCACTGAGGA |
| LGP2001 | ref3_ | 62 | GGAACATAAAAATGAATATT |
| | 25219 | | ACAAATATACCGCAAAGCAT |
| | | | GTAGAGTTGCTACACCAAGG |
| | | | GTCGGGACGTCCAAAAAAAC |
| | | | TCACTGAGGAAGTCGACTGG |
| | | | AAGCACGAGGCGCCCCCCCC |
| | | | AGGAGCGGGGCGACCGGCAA |
| | | | GGGGGCCCGCAATTGTCGCC |
| | | | ATGATCGACCAGCTTAGGTA |
| | | | GGATCCTCTTTCGACCTAAC |
| | | | GAATGGCTGCTTCTATTGGG |
| | | | GCAACGCCATTATACAATAT |
| | | | CGCCTGACCATCTGGAACGC |
| | | | GGCCCGGTCCACCGGCAGGT |
| | | | TGGCGACGACAGCGTCGGAG |
| LGP2001 | ref1_ | 63 | CGGCGTCGACCAGCCGGGCG |
| | 4361220 | | AACTGCTTGGGCATGCTCTC |
| | | | CCGCGACGCCGGCCACAGCC |
| | | | GCGTCCCCGTCCCTCCGCAC |
| | | | AGGATCATCGGGTGGATTTG |
| | | | AAAGGCAAAACGGGACATCA |
| | | | GGATAGGCCGCTCAGGCGTT |
| | | | GGCGCTGAGGCGCTTGATGT |
| | | | CGGCGTCGACCATCTCGGTG |
| | | | ATCAGCGCCTCGAGGCTGGT |
| | | | CTCGGCCTCCCAGCCGAAGG |
| | | | TCGCCTTGGCCTTGGCGGGG |
| | | | TTGCCCAGCAGCACCTCGAC |
| | | | CTCTGCCGGCCGGAACAGCG |
| | | | CCGGGTCGACGATCAGGTGG |
| LGP2001 | ref1_ | 64 | CTGGACATGCGCCCACCCCG |
| | 4602420 | | GCCAAGTCCGACCGCACCGG |
| | | | CAACCGCTCCTGTAGTCGTC |
| | | | GTCATCGTTCTCACCCCTGA |
| | | | GGCGGAGACCGTCCGCTAAC |
| | | | GGGGTGTCTCAAGCAACCGT |
| | | | GGGGCGGAGGAACACGCACG |
| | | | TAGTCGCGTTTCAAGGTTCG |
| | | | CACGAACGCCTCGGCCATGC |
| | | | CGTTGCTCTGCGGGCTCTCC |
| | | | AGCGGCGTCGTTTTTGGCAC |
| | | | CAAACCAAGGTCGCGGGCGA |
| | | | AGCGGCGCGTGTCGCGGGGA |
| | | | CTGTCAGGAATTTCGTGTGG |
| | | | GGGCGGCCATAGTGGATCCG |
| LGP2004 | ref1_ | 65 | GGAAATCGGCTTCAAGTACG |
| | 194299 | | ACGTCACGCCGGCCATGCAG |
| | | | GTCACGGGTGCACTGTTCAA |
| | | | TCTCGAGCGCGACAACCAGC |
| | | | CGTTCCCCTCGAACGTGGAG |
| | | | TCCGGCCTCGTCCTTGGCGC |
| | | | AGGTCAGACACGCACCCAGG |
| | | | GCGCGGAAATCGGCCTGGCC |
| | | | GGCTATCTAACCGATTGGTG |
| | | | GCAGGTCTTTGGCGGCTACG |
| | | | CTTATACCGAGGCACGCGTA |
| | | | CTCTCGCCACTGGAAGACGA |
| | | | TGGAGACGTGATCGCAGCAG |
| | | | GTAATCTCGTCGGCAACGTT |
| | | | CCGCTAAATACTTTCAGTCT |
| LGP2004 | ref1_ | 66 | CGGCCTGGCCGGCTATCTAA |
| | 194305 | | CCGATTGGTGGCAGGTCTTT |
| | | | GGCGGCTACGCTTATACCGA |
| | | | GGCACGCGTACTCTCGCCAC |
| | | | TGGAAGACGATGGAGACGTG |
| | | | ATCGCAGCAGGTAATCTCGT |
| | | | CGGCAACGTTCCGCTAAATA |
| | | | CTTTCAGTCTGTTCAACAAG |
| | | | TTCGATATCAACGAGAATTT |
| | | | CTCCGTTGCTCTGGGCTATT |
| | | | ACTATCAGGATGCCAGCTTT |
| | | | GCCTCCTCAGACAATGCAGT |
| | | | GCGTTTGCCAAGTTATTCGC |
| | | | GGTTCGATGGCGGGTTGTTC |
| | | | TATCGATTCGACGAGTTGAC |
| LGP2004 | ref1_ | 67 | ACGTTCCGCTAAATACTTTC |
| | 194310 | | AGTCTGTTCAACAAGTTCGA |
| | | | TATCAACGAGAATTTCTCCG |
| | | | TTGCTCTGGGCTATTACTAT |
| | | | CAGGATGCCAGCTTTGCCTC |
| | | | CTCAGACAATGCAGTGCGTT |
| | | | TGCCAAGTTATTCGCGGTTC |
| | | | GATGGCGGGTTGTTCTATCG |
| | | | ATTCGACGAGTTGACACGCG |
| | | | TTCAGCTTAGCGTCGAGAAC |
| | | | ATTTTCGACAGGCGTTACAT |
| | | | CATCAACTCCAACAACAACA |
| | | | ACAACCTCACGCCTGGCGCG |
| | | | CCGAGAACAGTCCGCGTGCA |
| | | | ATTGATCGCTCGGTTCTAAA |
| LGP2003 | ref1_ | 68 | AGCCCACAAGCCTGATGCAC |
| | 86157 | | TTAACTACATCCTCTAATGT |
| | | | CGCGCCAATTTGCTTGGCGG |
| | | | CAGGGGATGTTGTATCGTCA |
| | | | TAGGCTTGTCTAACCGGAAC |
| | | | TTGTTTGCCAATCTCTTTGG |
| | | | CGATCGCAACCGCCATCTCG |
| | | | TGTTCGTCAACCATGTGCGC |
| | | | GTTCCTCTAATTGCACTCAT |
| | | | GGTGCCACGTGCACCTCCGA |
| | | | TCGTCTCGTGTCTAGAATGA |
| | | | AGGTGGGAACAACCTTACAC |
| | | | AGGCTTTCGCGACGCGCGAA |
| | | | TTTCTGGTTTCTCCGCCTCG |
| | | | GATGTGGGTTTGAGCGCTTC |
| LGP2003 | ref1_ | 69 | CTTTTCATTTGTCATGATCT |
| | 142469 | | CGACCAAGGTATTCACGGCA |
| | | | AGCTCGGTCTGTTGCTTAGC |
| | | | AAGTGCCTGAACTTCGCGAA |
| | | | CGATCGGCTCTCGACCCTTC |
| | | | GGGTTCGAGACCTGTCCCTT |
| | | | TTGAAAACCACGTGCCCTAC |
| | | | ACTTTTCGGGATCAAGGTGC |
| | | | GGGTTGGCTTTGGTCAAAAT |
| | | | TCTCTGGCGTCCCATTACAC |
| | | | GCCCTCCGCATCATCGTTCC |
| | | | CGCGAACGATCTGACCCCCG |
| | | | ACTTCCGCGAGGAAGCGTGT |
| | | | GGCGTGATCCTCGAAGCGGA |
| | | | ATGCCACCTCGAACTGTTCC |
| LGP2003 | ref1_ | 70 | CAGCAGCAAGCAGATCGTTG |
| | 142321 | | AAAACCGCTTGAACCGCATC |
| | | | TTGATCGGGACCGGAACCAA |
| | | | TCAGGTCATCTAGGTAAACC |
| | | | GAGACGTAAACTCGTTTGCG |
| | | | CTCGGCATCTTTCAGAACGT |
| | | | CCGTGATGCCAGACCGCATT |
| | | | AGTACCATCGTCGCCAAGGC |
| | | | GGGCGACTGAACGAAGCCGA |
| | | | TCGGCAGAGAGTAACGGGGA |
| | | | CCGCCCCTAATCGGGTTGCG |
| | | | AACGCAAGACCACTTAGCAA |
| | | | AGGTTCGAGCACGGCCGAAC |
| | | | TTCGCATGGTGGAGAGCCGC |
| | | | GGCAACACGGTTCCGTGATA |
| LGP2009 | ref1_ | 71 | TAGACATTCCAACAAACCGG |
| | 153668 | | CAAGAGGCTCGTCCTCACTC |
| | | | GAGGATTTGTTGGGACTTGC |
| | | | ATGATGTCGAAGCGGAGCCG |
| | | | TTATGACCTGGGTGCGATCA |
| | | | TGCGCCGAGCATGGGAGATG |
| | | | GCTCGGGAGGCGGCATTCGC |
| | | | GGTTGGCGAGCGGGCACGGA |
| | | | CTCACCTTGCTGCCGCGATG |
| | | | CGCAGCGCGTGGGCCGAAGC |
| | | | CAAGTTGGCACTCGCGCCCA |
| | | | CGAAGACGGAGCAGGATCGT |
| | | | CTCTCTCCGAGCGACATGAT |
| | | | CGGACATGAGGACGCCTACC |
| | | | AAGGCCGGGTTCTAAAATAT |
| LGP2009 | ref1_ | 72 | AAGATGGATACGACAAGCGC |
| | 3842117 | | GATTACATTATTTGCGAAAT |
| | | | AGATGGACAAATAAAAGACA |
| | | | AAGGACTGATGTATTTCCTT |
| | | | AAATCTGGACAAGTTGACCT |
| | | | CTTTCACATAGAAGTCACCA |
| | | | CTCCCTTTGGGACAATTTGG |
| | | | TGTCACGAAAACATAGAGGC |
| | | | CGAACTTCTTAGCTGAATTA |
| | | | TCGCGCTCCGGGTTCTTATG |
| | | | CGGCTGAGTGAAGCGCGGGA |
| | | | CAGCTTGCGAGCAGGGCCGC |
| | | | CAATGGCAGCCGGGATGACA |
| | | | CAATGCTCGGTCTCCCGACG |
| | | | CTTCTTCAATCGGGAGCGCT |
| LGP2009 | ref1_ | 73 | AGCTGAATTATCGCGCTCCG |
| | 3842278 | | GGTTCTTATGCGGCTGAGTG |
| | | | AAGCGCGGGACAGCTTGCGA |
| | | | GCAGGGCCGCCAATGGCAGC |
| | | | CGGGATGACACAATGCTCGG |
| | | | TCTCCCGACGCTTCTTCAAT |
| | | | CGGGAGCGCTTCGCAGCCCG |
| | | | GGGCGGCGCGCTCATGCGTC |
| | | | ACGACCTGGGCCCTGCGCAC |
| | | | CTTCGCGGCCCCGCCGTCCC |
| | | | GGCAGATCCCTGATGCCCCA |
| | | | AGTGGGCGGCCACTCCATCA |
| | | | AAGAACCCCGGCCTGTGGCA |
| | | | GATCTCGTAGGCATACCGAG |
| | | | GTTCCGCAGTGCCCCCACC |
| LGP2020 | ref1_ | 74 | ACCGAAGGCGTCCCCGGACA |
| | 2810264 | | CGAAGGCCTGAAACACCATA |
| | | | TCTGTGGCGATCAGGCCGAC |
| | | | GTGGTCGCGGACTTCAACTG |
| | | | GCAGAGAATGCCAGGCCGCT |
| | | | TCGATTTCAGATGATACTGG |
| | | | TACGGACATAGGAGCGGCTT |
| | | | AGCTTTCTCAGTGCAAATGT |
| | | | GATTGATTCCGGCTCAAAAA |
| | | | TGATCTTGATCGGACGAGAC |
| | | | GTTTTCAATCCATGTCGTGT |
| | | | TGCCATCGCCGATCGGTGCG |
| | | | TCAAGAGACAGATGGCGCCG |
| | | | ACCGTAGATACGCGTTCGGG |
| | | | TTGCCCGCACCGCTTCTCCA |
| LGP2020 | ref1_ | 75 | GGAGGTGTGATCTGATGATG |
| | 322980 | | TGCTGGATGAAATTGGCGGT |
| | | | CGAGCACTTGTTCAGCTTGG |
| | | | CCAGCTCGACGAGATCGGCG |
| | | | TGATGCTCGGCGTCGATCAG |
| | | | GATGTTCAGCGAGACCGGAC |
| | | | GTACGCAGGACTTGGTATTA |
| | | | GCGCCGTTGCGCATCAGCTT |
| | | | GCAGCCTTGCTCTGCTTCTC |
| | | | AGCGTGCCGCGTCAGGATGA |
| | | | CCCTGATGTAGCTGTTGAGG |
| | | | TTGATGCCGTAATAGCCTGC |
| | | | GGACTCTGTGAGATCCCGGC |
| | | | GAAGATCGTCGGCGAGGGTC |
| | | | AGGCGGATGGTGCTGGTCGG |
| LGP2020 | ref1_ | 76 | AAGTAACCGCTCAACATGAT |
| | 2785241 | | CTTCAGCATGTTGTCCAACA |
| | | | GCAGGAGAATACATGTAATT |
| | | | CACCATGACCGGCAAGCTGC |
| | | | GACTGGCCATTGCTTCCACC |
| | | | GCTTGAATGTAGCGATCGAA |
| | | | TTTCGCAAAATCAGGGTGGA |
| | | | ATGAAAATATCGAACCAAAC |
| | | | TGCGAGCCTTGAATCCGTTC |
| | | | TGCAAAATTATCGAAAAATT |
| | | | TTCTTGGCCGACTGCCGTTC |
| | | | GAAAACATTCTTACGTTTAC |
| | | | ATGCGGCCCGCCTGAAACAA |
| | | | GACAGTCTACCAGCTCTGGG |
| | | | AAATGGGGGTGAAGGGTCGG |

### Example 4. Analysis of Effects of Methylobacterium Strains on Nutrient Content of Plant Vegetative Tissues

Soybean seeds treated as described in Example 1 were grown in multiple field locations in the Midwestern United States in the summer of 2019 in parallel with untreated control soybean plants. Seeds from Canola and wheat were similarly treated and tested. For analysis of field grown corn plants, Methylobacterium strains were applied in-furrow at planting. Strains and strain combinations evaluated are shown in Table 19 below.

TABLE 19

| Crop | Methylobacterium strain(s) |
|---|---|
| Soybean (+Rhizobia treatment) | LGP2009 |
| Soybean (+Rhizobia treatment) | LGP2020 |
| Soybean (+Rhizobia treatment) | LGP2016 |
| Soybean (+Rhizobia treatment) | LGP2002 + LGP2015 |
| Soybean | LGP2002 |
| Soybean | LGP2009 |
| Soybean | LGP2004 |
| Soybean | LGP2015 |
| Soybean | LGP2001 |
| Soybean | LGP2017 |
| Soybean | LGP2002 + LGP2015 |
| Soybean | LGP2019 |

Preliminary analysis of soybean vegetative tissue indicated increased micronutrients were obtained by treatment with Methylobacterium strains, including increased boron in R1 stage vegetative tissue in soybean plants grown from LGP2002 and LGP2017-treated seeds, and increased iron in V6 stage vegetative tissue in soybean plants grown from LGP2001-treated seeds.

LGP2002, LGP2017, LGP2001, LGP2016, LGP2019, and LGP2020 are tested to evaluate effects on micronutrient levels and growth enhancement of leafy green plants as described in Example 2.

### Example 5. Methylobacterium Growth Stimulation of Cannabis Plants

The ability of Methylobacterium isolates LGP2002, LGP2009, and LGP2019 to enhance rooting and growth of cannabis plants (Cannabis sativa L.) was evaluated as follows. Cuttings were taken from a mature plant and immersed for 2 hours in a suspension of Methylobacterium in water at a concentration of approximately 1×10⁶ CFU per ml. A control solution (water only) contained no Methylobacterium. The wounded stem portion of cuttings in both the control and Methylobacteirum treatments were then dipped in synthetic rooting hormone 0.3% indole-3-butyric acid (IBA) and inserted, stem down, into a potting media plug in a molt-plug tray. Fifty plants total, 10 of each of 5 different CBD oil cannabis varieties, were treated with each Methylobacterium isolate. After 2 weeks in the potting medium, plugs were non-destructively harvested and roots were scored using a visual rating scale of 1-5:1=between 0 and 20% visible roots; 2=between 21 and 40% visible roots; 3=between 41 and 60% visible roots; 4=between 61 and 80% visible roots; 5=between 81 and 100% visible roots.

Rooting scores for plants treated with the tested Methylobacterium isolates ranged from 3-3.4, compared to a score of 2.6 for the untreated control plants. Treatments with LGP2002 and LGP2019 resulted in increases that were significantly different from the control at p<0.05, and treatment with LGP2009 resulted in increases that were significantly different from the control at p<0.001.

The rooted plantlets were transplanted to the field. Aboveground biomass was harvested approximately thirteen weeks after transplanting and dried, and the aboveground dry biomass determined. Treatment with three Methylobacterium isolates, LGP2002, LGP2009, and LGP2019, resulted in increased aboveground dry biomass in comparison to the untreated control plants. Treatment with LGP2009 resulted in an 18% increase in aboveground dry biomass, treatment with LGP2002 resulted in a 27% increase in aboveground dry biomass, and treatment with LGP2019 resulted in a 38% increase in aboveground dry biomass, a difference that was significantly different from the control at p<0.05. Enhanced rooting as the result of treatment with Methylobacterium isolates can lead to earlier transplanting of plantlets to the field without negatively impacting yield, thus resulting in decreased cycling time.

### Example 6. Methylobacterium Growth Stimulation of Cannabis Plants

The ability of Methylobacterium isolates LGP2000 (NRRL B-50929), LGP2001 (NRRL B-50930), LGP2002 (NRRL B-50931), LGP2003 (NRRL B-50932), LGP2004 (NRRL B-50933), LGP2005 (NRRL B-50934), LGP2006 (NRRL B-50935), LGP2007 (NRRL B-50936), LGP2008 (NRRL B-50937), LGP2009 (NRRL B-50938), LGP2010 (NRRL B-50939), LGP2011 (NRRL B-50940), LGP2012 (NRRL B-50941), LGP2013 (NRRL B-50942), LGP2014 (NRRL B-67339), LGP2015 (NRRL B-67340), LGP2016 (NRRL B-67341), LGP2017 (NRRL B-67741), LGP2018 (NRRL B-67742), LGP2019 (NRRL B-67743), LGP2020 (NRRL B-67892), LGP2021 (NRRL B-68032), LGP2022 (NRRL B-68033), LGP2023 (NRRL B-68034), LGP2029 (NRRL B-68065), LGP2030 (NRRL B-68066), LGP2031 (NRRL B-68067), LGP2033 (NRRL B-68068), LGP2034 (NRRL B-68069), and LGP2167 (NRRL B-67927) to enhance rooting and growth of cannabis plants (Cannabis sativa L.) are evaluated as follows. Cuttings are taken from a mature plant and immersed for 2 hours in a suspension of Methylobacterium in water at a concentration of approximately 1×10⁶ CFU per ml. A control solution (water only) contains no Methylobacterium. The wounded stem portion of cuttings in both the control and Methylobacteirum treatments are then dipped in synthetic rooting hormone 0.3% indole-3-butyric acid (IBA) and are inserted, stem down, into a potting media plug in a mult-plug tray. Fifty plants total, 10 of each of 5 different CBD oil cannabis varieties, are treated with each Methylobacterium isolate. After 2 weeks in the potting medium, plugs are non-destructively harvested and roots were scored using a visual rating scale of 1-5:1=between 0 and 20% visible roots; 2=between 21 and 40% visible roots; 3=between 41 and 60% visible roots; 4=between 61 and 80% visible roots; 5=between 81 and 100% visible roots.

Rooting scores for plants treated with the tested Methylobacterium isolates are determined as compared to the untreated control plants. The rooted plantlets are transplanted to the field. Aboveground biomass is harvested approximately thirteen weeks after transplanting and dried, and the aboveground dry biomass is determined.

### Example 7. Methylobacterium Inoculation Effect on Promotion of Early Rice Growth

Methylobacterium isolates were tested for their ability to enhance early growth of rice seedlings. A randomized complete block design was used, with 12 treatments in each run; 10 unique Methylobacterium isolates, a Methylobacterium positive control, LGP2018, that demonstrated consistent root growth promotion of rice seedlings during assay development and increased yield levels in corn field trials (WO2020117690). The untreated control sample (UTC) was Methylobacterium growth medium applied in the same amount as used for the Methylobacterium isolates. Each treatment level had an n of 10. All 10 blocks were grown in the same growth chamber and on the same shelf.

### Procedure:

### Media:

0.5× Murashige and Skoog MS agar plates with 0.5% sucrose

### Pre-Planting:

Rice seeds were de-husked. Average 100 seed count is 2018 mg with approximately 21 g of husked rice per run.

### Planting:

Seeds were sterilized in ˜3% sodium hypochlorite+0.05% Tween 20.
Seeds were washed to remove bleach solution and placed on a sterile plate lid to begin drying.
Seeds were plated using a randomized complete block design with each complete block having similarly sized seeds.
Using sterile techniques 8 sterile seeds were evenly spaced in a horizontal line (˜ 40% above the bottom of the plate, using a pre-marked lid as a guide). Seeds were placed with the embryo toward the bottom of the plate and gently pushed into media.

### Inoculation:

Each Methylobacterium isolate or the culture medium control was applied as an 80 uL streak to the bottom portion of the plate (one isolate per plate) and spread by gently tilting the plate back and forth. A target concentration of 1×10⁶ CFU per seed was applied.
Plates were allowed to dry for at least on hour and placed in a randomized layout in a Percival growth chamber set to 25° C. and 16 hour days.
Seeds were allowed to grow undisturbed for 8 days.

### Harvest:

At 8 days after plating the plates were removed from the growth chambers, and the plants (approximately V2 stage) were measured as follows.
Plants that were not impeded from growing normally (by physical surroundings unrelated to presence of Methylobacterium) were removed from plates, and the number of seedlings for that plate was recorded.
Seedlings were scanned using WinRhizo and the images analyzed to determine root length for each plant.

The results of this experiment are shown below in Table 20.

TABLE 20

| | | | Absolute | Normalized |
|---|---|---|---|---|
| Experiment | | | Root | Root |
| Number | Treatment ID | Treatment | Length (cm) | Length |
| 264PB | 264PB LGP2018 | LGP2018 | 18.82978 | 100 |
| 264PB | 264PB Strain 1 | LGP2025 | 17.39133 | 73.325898 |
| 264PB | 264PB Strain 2 | LGP2073 | 17.19 | 69.59247 |
| 264PB | 264PB Strain 3 | LGP2047 | 16.37316 | 54.44538 |
| 264PB | 264PB Strain 4 | LGP2045 | 15.96066 | 46.796074 |
| 264PB | 264PB Strain 5 | LGP2151 | 15.39851 | 36.371618 |
| 264PB | 264PB Strain 6 | LGP2103 | 15.04489 | 29.814374 |
| 264PB | 264PB Strain 7 | LGP2125 | 14.84019 | 26.018352 |
| 264PB | 264PB Strain 8 | LGP2017 | 14.54892 | 20.61718 |
| 264PB | 264PB Strain 9 | LGP2120 | 13.84252 | 7.517937 |
| 264PB | 264PB Strain 10 | LGP2124 | 13.18279 | −4.715877 |
| 265PB | 265PB Strain 1 | LGP2071 | 14.117796 | 100.010863 |
| 265PB | 265PB LGP2018 | LGP2018 | 14.117132 | 100 |
| 265PB | 265PB Strain 2 | LGP2061 | 12.535499 | 74.124179 |
| 265PB | 265PB Strain 3 | LGP2107 | 11.83976 | 62.741755 |
| 265PB | 265PB Strain 4 | LGP2065 | 9.992807 | 32.52525 |
| 265PB | 265PB Strain 5 | LGP2051 | 9.743358 | 28.444232 |
| 265PB | 265PB Strain 6 | LGP2054 | 8.960485 | 15.636268 |
| 265PB | 265PB Strain 7 | LGP2092 | 8.856461 | 13.934427 |
| 265PB | 265PB Strain 8 | LGP2079 | 8.610079 | 9.903568 |
| 265PB | 265PB Strain 9 | LGP2052 | 7.916505 | −1.443435 |
| 266PB | 266PB Strain 1 | LGP2059 | 15.569966 | 123.451522 |
| 266PB | 266PB Strain 2 | LGP2016 | 14.587924 | 108.443799 |
| 266PB | 266PB LGP2018 | LGP2018 | 14.035398 | 100 |
| 266PB | 266PB Strain 3 | LGP2158 | 13.207394 | 87.346316 |
| 266PB | 266PB Strain 4 | LGP2066 | 12.900975 | 82.663567 |
| 266PB | 266PB Strain 5 | LGP2141 | 11.897894 | 67.334339 |
| 266PB | 266PB Strain 6 | LGP2078 | 10.298694 | 42.8951 |
| 266PB | 266PB Strain 7 | LGP2050 | 10.041706 | 38.967777 |
| 266PB | 266PB Strain 8 | LGP2080 | 9.462625 | 30.118161 |
| 266PB | 266PB Strain 9 | LGP2048 | 9.284123 | 27.390276 |
| 266PB | 266PB Strain 10 | LGP2053 | 7.207347 | −4.347354 |
| 267PB | 267PB Strain 1 | LGP2046 | 14.419073 | 137.78678 |
| 267PB | 267PB LGP2018 | LGP2018 | 12.303465 | 100 |
| 267PB | 267PB Strain 2 | LGP2024 | 11.846345 | 91.835407 |
| 267PB | 267PB Strain 3 | LGP2148 | 10.620679 | 69.94383 |
| 267PB | 267PB Strain 4 | LGP2144 | 9.415631 | 48.420528 |
| 267PB | 267PB Strain 5 | LGP2150 | 9.382432 | 47.827557 |
| 267PB | 267PB Strain 6 | LGP2110 | 9.298016 | 46.319801 |
| 267PB | 267PB Strain 7 | LGP2176 | 8.103827 | 24.990443 |
| 267PB | 267PB Strain 8 | LGP2153 | 7.128328 | 7.567103 |
| 267PB | 267PB Strain 9 | LGP2082 | 6.373293 | −5.91855 |
| 268PB | 268PB Strain 1 | LGP2021 | 15.569966 | 123.451522 |
| 268PB | 268PB Strain 2 | LGP2040 | 14.587924 | 108.443799 |
| 268PB | 268PB LGP2018 | LGP2018 | 14.035398 | 100 |
| 268PB | 268PB Strain 3 | LGP2138 | 13.207394 | 87.346316 |
| 268PB | 268PB Strain 4 | LGP2095 | 12.900975 | 82.663567 |
| 268PB | 268PB Strain 5 | LGP2130 | 11.897894 | 67.334339 |
| 268PB | 268PB Strain 6 | LGP2099 | 10.298694 | 42.8951 |
| 268PB | 268PB Strain 7 | LGP2077 | 10.041706 | 38.967777 |
| 268PB | 268PB Strain 8 | LGP2102 | 9.462625 | 30.118161 |
| 268PB | 268PB Strain 9 | LGP2072 | 9.284123 | 27.390276 |
| 268PB | 268PB Strain 10 | LGP2081 | 7.207347 | −4.347354 |
| 269PB | 269PB LGP2018 | LGP2018 | 16.079324 | 100 |
| 269PB | 269PB Strain 1 | LGP2094 | 15.70514 | 95.501874 |
| 269PB | 269PB Strain 2 | LGP2101 | 15.386634 | 91.673054 |
| 269PB | 269PB Strain 3 | LGP2090 | 14.624067 | 82.506105 |
| 269PB | 269PB Strain 4 | LGP2093 | 12.998755 | 62.967937 |
| 269PB | 269PB Strain 5 | LGP2084 | 12.830224 | 60.942001 |
| 269PB | 269PB Strain 6 | LGP2114 | 12.516872 | 57.175138 |
| 269PB | 269PB Strain 7 | LGP2100 | 11.343389 | 43.068489 |
| 269PB | 269PB Strain 8 | LGP2085 | 9.828333 | 24.855728 |
| 269PB | 269PB Strain 9 | LGP2075 | 7.587342 | −2.08362 |
| 269PB | 269PB Strain 10 | LGP2083 | 7.50976 | −3.016248 |
| 270PB | 270PB Strain 1 | LGP2029 | 14.570904 | 104.017951 |
| 270PB | 270PB LGP2018 | LGP2018 | 14.31934 | 100 |
| 270PB | 270PB Strain 2 | LGP2135 | 13.363759 | 84.737607 |
| 270PB | 270PB Strain 3 | LGP2129 | 12.594344 | 72.448632 |
| 270PB | 270PB Strain 4 | LGP2143 | 10.608781 | 40.735534 |
| 270PB | 270PB Strain 5 | LGP2137 | 10.04973 | 31.806444 |
| 270PB | 270PB Strain 6 | LGP2128 | 9.970479 | 30.540667 |
| 270PB | 270PB Strain 7 | LGP2123 | 9.933589 | 29.951459 |
| 270PB | 270PB Strain 8 | LGP2126 | 9.635704 | 25.193695 |
| 270PB | 270PB Strain 9 | LGP2136 | 9.506136 | 23.124249 |
| 270PB | 270PB Strain 10 | LGP2121 | 7.872883 | −2.961817 |
| 271PB | 271PB LGP2018 | LGP2018 | 18.545695 | 100 |
| 271PB | 271PB Strain 1 | LGP2069 | 16.856945 | 83.10707 |
| 271PB | 271PB Strain 2 | LGP2027 | 15.948911 | 74.02381 |
| 271PB | 271PB Strain 3 | LGP2056 | 14.750148 | 62.03233 |
| 271PB | 271PB Strain 4 | LGP2096 | 14.330543 | 57.83493 |
| 271PB | 271PB Strain 5 | LGP2060 | 13.874818 | 53.27622 |
| 271PB | 271PB Strain 6 | LGP2097 | 13.443795 | 48.9646 |
| 271PB | 271PB Strain 7 | LGP2067 | 13.24211 | 46.9471 |
| 271PB | 271PB Strain 8 | LGP2055 | 12.770669 | 42.23118 |
| 271PB | 271PB Strain 9 | LGP2086 | 12.549608 | 40.01986 |
| 271PB | 271PB Strain 10 | LGP2057 | 11.572393 | 30.24456 |
| 273PB | 273PB LGP2018 | LGP2018 | 13.216513 | 100 |
| 273PB | 273PB Strain 1 | LGP2028 | 11.289892 | 71.38989 |
| 273PB | 273PB Strain 2 | LGP2098 | 10.957287 | 66.45074 |
| 273PB | 273PB Strain 3 | LGP2116 | 10.552009 | 60.43241 |
| 273PB | 273PB Strain 4 | LGP2131 | 10.492209 | 59.54438 |
| 273PB | 273PB Strain 5 | LGP2117 | 9.92343 | 51.09808 |
| 273PB | 273PB Strain 6 | LGP2133 | 9.207299 | 40.46361 |
| 273PB | 273PB Strain 7 | LGP2140 | 9.188468 | 40.18397 |
| 273PB | 273PB Strain 8 | LGP2134 | 8.651127 | 32.20451 |
| 273PB | 273PB Strain 9 | LGP2109 | 7.244746 | 11.31992 |
| 273PB | 273PB Strain 10 | LGP2111 | 5.404409 | −16.0089 |
| 274PB | 274PB Strain 1 | LGP2033 | 17.459903 | 136.108331 |
| 274PB | 274PB Strain 2 | LGP2118 | 15.623786 | 106.167536 |
| 274PB | 274PB LGP2018 | LGP2018 | 15.245562 | 100 |
| 274PB | 274PB Strain 3 | LGP2145 | 14.631981 | 89.994584 |
| 274PB | 274PB Strain 4 | LGP2032 | 14.299443 | 84.572029 |
| 274PB | 274PB Strain 5 | LGP2152 | 13.881329 | 77.754029 |
| 274PB | 274PB Strain 6 | LGP2147 | 13.409769 | 70.064484 |
| 274PB | 274PB Strain 7 | LGP2157 | 11.306689 | 35.770445 |
| 274PB | 274PB Strain 8 | LGP2142 | 10.1196 | 16.413079 |
| 274PB | 274PB Strain 9 | LGP2159 | 9.361136 | 4.045128 |
| 274PB | 274PB Strain 10 | LGP2154 | 8.943802 | −2.760155 |
| 275PB | 275PB LGP2018 | LGP2018 | 18.826053 | 100 |
| 275PB | 275PB Strain 1 | LGP 2022 | 17.00802 | 80.576456 |
| 275PB | 275PB Strain 2 | LGP2023 | 16.310993 | 73.129541 |
| 275PB | 275PB Strain 3 | LGP2160 | 15.87016 | 68.41976 |
| 275PB | 275PB Strain 4 | LGP2163 | 15.337422 | 62.728087 |
| 275PB | 275PB Strain 5 | LGP2167 | 15.162438 | 60.858589 |
| 275PB | 275PB Strain 6 | LGP2166 | 14.298438 | 51.627764 |
| 275PB | 275PB Strain 7 | LGP2161 | 13.02194 | 37.989883 |
| 275PB | 275PB Strain 8 | LGP2162 | 11.85523 | 25.52496 |
| 275PB | 275PB Strain 9 | LGP2168 | 10.190812 | 7.742619 |
| 277PB | 277PB LGP2018 | LGP2018 | 15.854562 | 100 |
| 277PB | 277PB Strain 1 | LGP2062 | 14.420103 | 81.45296 |
| 277PB | 277PB Strain 2 | LGP2185 | 14.124727 | 77.63385 |
| 277PB | 277PB Strain 3 | LGP2063 | 13.598758 | 70.83327 |
| 277PB | 277PB Strain 4 | LGP2074 | 12.56993 | 57.53088 |
| 277PB | 277PB Strain 5 | LGP2058 | 12.237293 | 53.23002 |
| 277PB | 277PB Strain 6 | LGP2064 | 11.790611 | 47.45458 |
| 277PB | 277PB Strain 7 | LGP2091 | 11.598483 | 44.97043 |
| 277PB | 277PB Strain 8 | LGP2186 | 10.193847 | 26.809 |
| 277PB | 277PB Strain 9 | LGP2105 | 10.166668 | 26.45758 |
| 277PB | 277PB Strain 10 | LGP2187 | 10.018778 | 24.54541 |
| 282PB | 282PB LGP2018 | LGP2018 | 17.115992 | 100 |
| 282PB | 282PB Strain 1 | LGP2087 | 15.150588 | 77.27183 |
| 282PB | 282PB Strain 2 | LGP2108 | 14.929319 | 74.71305 |
| 282PB | 282PB Strain 3 | LGP2076 | 14.913514 | 74.53028 |
| 282PB | 282PB Strain 4 | LGP2106 | 13.131888 | 53.92734 |
| 282PB | 282PB Strain 5 | LGP2113 | 12.547632 | 47.17093 |
| 282PB | 282PB Strain 6 | LGP2049 | 12.529399 | 46.96009 |
| 282PB | 282PB Strain 7 | LGP2068 | 12.507406 | 46.70576 |
| 282PB | 282PB Strain 8 | LGP2149 | 12.28271 | 44.10735 |
| 282PB | 282PB Strain 9 | LGP2005 | 11.888991 | 39.55433 |
| 282PB | 282PB Strain 10 | LGP2006 | 10.285192 | 21.00781 |
| 283PB | 283PB Strain 1 | LGP2182 | 14.59702 | 103.904114 |
| 283PB | 283PB LGP2018 | LGP2018 | 14.364828 | 100 |
| 283PB | 283PB Strain 2 | LGP2034 | 13.842152 | 91.211673 |
| 283PB | 283PB Strain 3 | LGP2146 | 12.351052 | 66.14017 |
| 283PB | 283PB Strain 4 | LGP2181 | 12.117376 | 62.211111 |
| 283PB | 283PB Strain 5 | LGP2089 | 11.13865 | 45.754717 |
| 283PB | 283PB Strain 6 | LGP2156 | 10.858914 | 41.051207 |
| 283PB | 283PB Strain 7 | LGP2170 | 10.110786 | 28.472101 |
| 283PB | 283PB Strain 8 | LGP2155 | 9.582397 | 19.587708 |
| 283PB | 283PB Strain 9 | LGP2127 | 8.857205 | 7.394253 |
| 283PB | 283PB Strain 10 | LGP2139 | 8.755959 | 5.691884 |
| 285PB | 285PB LGP2018 | LGP2018 | 12.031742 | 100 |
| 285PB | 285PB Strain 1 | LGP2173 | 11.21333 | 84.0138457 |
| 285PB | 285PB Strain 2 | LGP2172 | 10.228408 | 64.7752232 |
| 285PB | 285PB Strain 3 | LGP2164 | 9.964949 | 59.6290516 |
| 285PB | 285PB Strain 4 | LGP2165 | 9.033842 | 41.4416163 |
| 285PB | 285PB Strain 5 | LGP2008 | 7.982016 | 20.8961413 |
| 285PB | 285PB Strain 6 | LGP2112 | 7.609441 | 13.6186008 |
| 285PB | 285PB Strain 7 | LGP2169 | 7.485808 | 11.2036581 |
| 285PB | 285PB Strain 8 | LGP2044 | 7.402148 | 9.5695127 |
| 285PB | 285PB Strain 9 | LGP 2011 | 6.922695 | 0.2042973 |
| 285PB | 285PB Strain 10 | LGP2171 | 5.864521 | −20.4651746 |
| 286PB | 286PB Strain 1 | LGP2001 | 18.47052 | 102.4019 |
| 286PB | 286PB LGP2018 | LGP2018 | 18.29094 | 100 |
| 286PB | 286PB Strain 2 | LGP2012 | 17.23022 | 85.81258 |
| 286PB | 286PB Strain 3 | LGP2000 | 17.06282 | 83.57344 |
| 286PB | 286PB Strain 4 | LGP2015 | 16.97065 | 82.34073 |
| 286PB | 286PB Strain 5 | LGP2007 | 15.82329 | 66.99432 |
| 286PB | 286PB Strain 6 | LGP2003 | 14.07074 | 43.5534 |
| 286PB | 286PB Strain 7 | LGP2010 | 14.04739 | 43.24119 |
| 286PB | 286PB Strain 8 | LGP2013 | 13.72635 | 38.9471 |
| 286PB | 286PB Strain 9 | LGP2004 | 12.51197 | 22.7044 |
| 288PB | 288PB Strain 1 | LGP2031 | 11.73032 | 115.04974 |
| 288PB | 288PB LGP2018 | LGP2018 | 10.961572 | 100 |
| 288PB | 288PB Strain 2 | LGP2030 | 10.823393 | 97.29486 |
| 288PB | 288PB Strain 3 | LGP2184 | 10.428576 | 89.56555 |
| 288PB | 288PB Strain 4 | LGP2188 | 10.060309 | 82.35601 |
| 288PB | 288PB Strain 5 | LGP2132 | 10.004185 | 81.25727 |
| 288PB | 288PB Strain 6 | LGP2179 | 9.603427 | 73.41165 |
| 288PB | 288PB Strain 7 | LGP2183 | 9.371095 | 68.86329 |
| 288PB | 288PB Strain 8 | LGP2122 | 8.820766 | 58.08953 |
| 288PB | 288PB Strain 9 | LGP2009 | 7.664263 | 35.44871 |
| 288PB | 288PB Strain 10 | LGP2088 | 6.600541 | 14.62428 |
| 289PB | 289PB Strain 1 | LGP2002 | 16.64733 | 117.25169 |
| 289PB | 289PB LGP2018 | LGP2018 | 15.73919 | 100 |
| 289PB | 289PB Strain 2 | LGP2174 | 14.52193 | 76.87615 |
| 289PB | 289PB Strain 3 | LGP2178 | 14.47025 | 75.89433 |
| 289PB | 289PB Strain 4 | LGP2119 | 14.41787 | 74.89923 |
| 289PB | 289PB Strain 5 | LGP2070 | 14.39551 | 74.47451 |
| 289PB | 289PB Strain 6 | LGP2104 | 14.2175 | 71.09291 |
| 289PB | 289PB Strain 7 | LGP2175 | 13.17078 | 51.20856 |
| 289PB | 289PB Strain 8 | LGP2115 | 13.15135 | 50.83953 |
| 289PB | 289PB Strain 9 | LGP2177 | 13.0369 | 48.66526 |
| 289PB | 289PB Strain 10 | LGP2180 | 13.00762 | 48.10911 |

Forty-eight Methylobacterium strains were selected for gene correlation analysis from the 176 strains tested, including 15 non-hits and 33 hits. The strains were selected from those having the highest and lowest normalized root scores, excluding any isolates that had any signs of any type of microbial contamination. The normalized score standardized each isolate's mean root length value to the UTC (a value of 0) and the positive control LGP2018 (a value of 100).

Genomes of the selected isolates were assembled and putative genes identified. The genes were assigned a putative function by sequence analysis to databases of known genes and gene signatures. A pan-genome for Methylobacterium was constructed as described by Page et al. (Roary: rapid large-scale prokaryote pan genome analysis, Bioinformatics. (2015) 31:3691-3693) except that genome sequences from greater than 1000 different species of Methylobacterium were assembled and used to construct the pan-genome as opposed to the single Salmonella species described by Page et al.

The genomes of strains identified as enhancing rice seedling growth, “hits”, and strains identified as “non-hits” were compared to determine the presence or absence in each strain of each genetic element in the pan-genome. For this analysis, translated genes were clustered across strains using BLASTP with a sequence identity of at least 50% to identify homologous genetic elements across genomes. These results were used to determine which genetic elements are the same or different across strains, leading to a score for each genetic element as present or absent in a given strain. The presence/absence scores were used in a correlation analysis to identify genetic elements that correlate positively with enhancing rice seedling growth as described by Brynildsrud et al. (Rapid scoring of genes in microbial pan-genome-wide association studies with Scoary, Genome Biology (2016) 17:238).

The steps in the process were as follows. Correlated genetic elements were collapsed so that genes that are typically inherited together, for example genes on the same plasmid, were combined into a single unit. Each genetic element in the pan-genome received a null hypothesis of no association to the trait. A Fisher's exact test was performed on each genetic element with the assumption that all strains had a random and independently distributed probability for exhibiting each state, i.e. presence or absence of the genetic element. To control spurious associations due to population structure, the pairwise comparisons algorithm was applied using a phylogenetic tree of the Methylobacterium genus, constructed using the same genome sequences described above. Empirical p-value was computed using label-switching permutations, i.e. the test statistic was generated over random permutations of the phenotype data. The genetic elements that were significantly positively correlated with enhancing rice seedling root growth were identified based on p value using a threshold for statistical significance of p less than or equal to 0.05. Sensitivity and specificity cutoffs were also employed based on the number of hits and non-hits a gene was present in.

Gene elements that were positively correlated with Methylobacterium enhancement of growth in rice seedlings are shown in Table 21 below.

TABLE 21

| | Consensus | | | | | |
|---|---|---|---|---|---|---|
| | Protein | Representative | | | | |
| Gene | SEQ ID | protein | | | | p- |
| name | NO: | sequences | Annotation | Sensitivity | Specificity | value |
| group_{—} | 77 | SEQ 84 | hypothetical | 60.61 | 80.00 | 0.003 |
| 4403 | | | protein | | | |
| group_{—} | 78 | SEQ 85 | hypothetical | 57.58 | 86.67 | 0.025 |
| 9931 | | | protein | | | |
| group_{—} | 79 | SEQ 86 | hypothetical | 66.67 | 86.67 | 0.030 |
| 7199 | | | protein | | | |
| recD2_2 | 80 | SEQ 87 | ATP-dependent | 45.45 | 93.33 | 0.035 |
| | | | RecD-like DNA | | | |
| | | | helicase | | | |
| pinR | 81 | SEQ 88 | Putative DNA- | 69.70 | 80.00 | 0.039 |
| | | | invertase from | | | |
| | | | lambdoid prophage | | | |
| | | | Rac | | | |
| group_{—} | 82 | SEQ 89 | hypothetical | 33.33 | 100.00 | 0.055 |
| 2780 | | | protein | | | |
| group_{—} | 83 | SEQ 90 | hypothetical | 60.61 | 80.00 | 0.057 |
| 5546 | | | protein | | | |

Methylobacterium consensus protein sequences for the above identified genes that positively correlate with enhanced growth or rice seedlings are provided as SEQ ID NO: 77 through SEQ TD NO: 83 below. Consensus sequences are generated by aligning the encoded protein sequences from all isolates from a comprehensive database of Methylobacterium genome sequences from public and internal databases. EMBOSS cons was used to generate consensus sequences from the multiple sequence alignment. Where no consensus was found at a position an ‘x’ character is used. An upper case letter for an amino acid residue indicates that most of the sequences have that amino acid at that position. In the consensus sequences, X can be any amino acid residue or can be absent.

SEQ ID NO. 77
xxxxxxxxxxxxxxxxxxxxxxxxxxxxxxxxxxxxxxxxxxxxx
xxxxxxxMPTxLPxxxxxxxxRxxPVRRLSWPDTARFLILVARVR
LLDxxxxxxxxxxxxxxxxxxxxxxxxxxxxxxxLRLHAxxxxxx
xxxxxVxRxGSxxAGDxLLxLMRRWLAxHEAIxALLPGVPEPxHV
AQVxxxxxxxxxxxxxxxxxxxxxxRAILQxxxxxxxxxVPxSRx
xxxxPxxxxxxxxxxxxxxxxxxxxxxxxxxxxxxxxxxxx
SEQ ID NO. 78
xxxxxxxMxxPLRRTVQVxEDGRMNLPADMRRVLGLTGAGRVILT
QDEDGIxITTaEQALKRVRSLAAPFxRGxGSVVDEFIAERRADAA
REDxExxxxxxxxxxxxxxxxxxxxxxxxxxxxxxxxxxxxxxxx
xxxxxxxxxxxxxxxxxxx
SEQ ID NO. 79
MxxxxxxxxxxxxxxxxxxxxxxxxxxxxxPQSYALQILAIAxAM
SVLGLGGVWIASRIYDRNTRRLEAxxxxRRGDxxxxxxxxxxxxx
xxxxxxxxxxxxxxx
SEQ ID NO. 80
xxxxxxxDTLExxxxxxxxxxxxxxxxRxxxxxLA
CTVxDHxSIAxxQNxVPIIRDIxLxNxxDxDLADVxLxIxAxPxL
xRPLTLxIxRIxAGxxxxIDxPDLRIDxAILxxxxxAGxxESxxx
xVTLxLxxSxxxxxxxxExARExxDLRLLPPSHWGGxxAAPELLA
AFVRPNDPAVDxILRxAAxILxRAxRxTAxxDGYxSGRKARAWEM
AEAIxAxxxxxAMAxxxxxxxxxxxxRIxxxxxxYVLPPASFERS
GQKVRxPxxIVERRLxTCLDLTLLWAACxEQAGLNPLLVLTxxHA
xLGLWLxDExxxxxxxDDxQxLRKRRDLQExxxxxxxxxxxxLIL
IETTILTxxxxxxxxxDPPxxFxxAxxxGAxxIDxDAxAxLEMxL
DLRRxRxxGIxPLDxGExxxxxxxxAPxxxxxxxxLxxxQxLxxx
xxxxxxAPPSFxEDxxxxxIDxxxxxxxxRLExWKxRLLDLTLRN
KLLNFKPGKGSLTLDCxEPGAxEDxLxAGxxFRLxxRPxxxxxDx
xxxxxxxxxxKxxxxxxxxxxAxxxRxEIxxxxxxxxxxxxxxxx
xxELExRLxDLFRLARxxFEEGGANVLFLAxGFLTWTRxxGxxxx
xRAPLLLVPxALxRASVRAGFRLxxHDEExRLNPTLLEMLRQDFx
LxMPDxxxxLPxDxSGIDVExIWRIVRTHIRDLKGWEVxxEVVLS
AFSFTKFLMWKDLxERxDLLKRSPVVRHLLDTPKAYGDGxxxTxF
PxPxRLDxEHPPxxIFxxxxxPLADSSQLSAILAAASGKDFVLFG
PPGTGKSxxxxxxxxxxQTIxNMIAQCLAxxGRTVLFVSQKSAAL
EVVxxRRRLxxVGLGxxCLEVHAxKAQKTxVIxQLREAWxxRxxx
xxxxWDxAxxDLxxxRExLNGVVxSLHxxRxNGLSAHxAxGRVIA
xxxxGxxxxLxLxWPxxxxxxxxxxxxSLxxxxxRAxCxELxxxx
xLxxxVGxIxDHPLRGIxAxxWSPLWRxEMxxAlxxLxRTLxxxx
xSGQxxAEAMGLxxLxxTYxGxxRxLxxLxxxLxRxEARxGLxFL
xxGxxxLRQAVxARxxxQxxxARLxxRLxxxYxxPxVxxxDLxxL
LAEWxxAKxSNFxLRGxRLxRVxxxLxPFAQGxxPxDIGPDLxxL
xEIxxxxxxxxxxxVxExxxAxLGxxxPxxxxWSDPxxPAxxFxA
xMAWAxRLxxVIxxMxPLxxxGxDxVRxxLxxxxxxLDxExxxLx
xxxxxxxxxxGGxLAxAxxxFxxxRxxAVKAIExLGRxxxxxxxx
xLAGRAxPDxxxxPVxxExxxxxxxxxxxxxxxxxxxxxxxxxxx
xxxxxxxxxxxxxxxxxxxxxxxxxxxxxxxxxxxxxxxxxxxxx
xxxxxxxxxxxxxxxxxxxxxxxxxxxxxxxxDxWVxxTLAVAxR
WxxxLxxKAQxWxAWQxAAxxAxKAGLxPLVxAIExGxIxxDxxx
xAFExAYARWWIDxxxTDDxxLRxxxxxFMxQRHEEAIRxFxxAD
SRLSxLAxxxVRARxxxxxxxIGGGVPxxxxxxxAxAFGxDPEWG
TLAxElxxxxxTKRxRHMPLRQLFxRMPNALTRLxxxTPCLMMSP
LSIAQYxPxExKPFDIVIFDEASQIAPWDAIGAIARGROVVIVGD
PEQLPPTNVGDRGVDEIxxxxDGxDVADQESILDECLAANLPQRx
LxxxxxWHYRSRHESLIAFSNxHYYxGxLVTFPSPVTDDxRAVRL
xxVxDGLYERGxxRVNRPEARALVAEVVxRLxDPxxxxxxxxxAF
AxExRSLGIVTENGEQQRLIENLLDxERRxxxxPELExFFDxxxW
xEPVFVKNLExVQGDERDAILFSVAxGPxxDxTGRxxxxISSLNR
EGGHxxxRRLNVAITRARRELVVFASMRxDQVDLGRxxARGVRDF
KHFLxFAExxGAxALxxAxAPTGGDIESPFExAVMAxxxxxxxxA
LxARGWxIxxQVGVSxFRIDLGIVHPDAPGRYLAGVECDGATYxx
xHxAATARDRDRLRExVLTDLGWRIxRVWSTDWWxDxQGALxRLD
xxLRxDLDADRAKxxxxxxxxxxxxxxxxxxxxxxxxxxxxxxxx
xxxxxxxxxxxxxxxxxxxxxxxxxxPxxxxxxxxxxxxPxxxxx
xxQxxxxPxxxxxxxxxxxxxxxxxYxxADLSxxGxxxDxxRFHD
xxYxxxLAAMxAxVVxxEGPVFxDILxxRLxRAHGxxRITxxLRQ
xxLxxVDPxxxxTxExxRIVLWPxGxxPxxxxxxFRPAxxxxxxx
xxxRAxxxDxPLxELxGLARxLxxxxxxxxxxxxMAxRLxxxxxx
xxxGLxRMxxAxxxxxxxxxxxxxxxxxxxxxxxxxxxxxxxRAR
FAEAxAxLxARESxxxxxxxxxxxxxxxxxxxxxxxxxxxxxxxx
xxxxxxxxxxxxxxxxxxxxxxxxxxxxxxxxxxxxxxxxxxxxx
xxxx
SEQ ID NO. 81
xxxxxxxxxxxxxxxxxxxxxxxxxxxxxxxxxxxxxxxxxxxxx
xxxxxxxxxxxxxxxxxxxxxxxxxxxxxxxxxxxxxxxMQTILY
ARVSTADQTIAHQRxQAEAAGFKIxDxVVADEGVSGVSTxLxDRP
QGRRLFDxxMLRRGDVLxxxxxxxVVRWVDRLGRNYAxxxxxxxx
xxxxxxxxxxxxxxxxxxxxxxxxxxxxDVTETIREFMRxxxxxx
xRGVIVRTVINNxxxxxxxxxxMTFDGATTDPMQxAVRDALxxxI
GFMAATAQAQAEATxKEAQKAGIEHAKxRxxExDxxAYRGRKPSY
TREQxxxDxVRxxLxQGxxxVSAIAKATGLSRQxTVYRIRDNPAE
AEAALARxxxxxxxxxxxxxxxWAAxxxxxxxxxxxxxxxxxxxx
xxxx
SEQ ID NO. 82
MxxxxxxxxxxxxxxxxxxYDDxxxADAAAGEERDAIMRALAEDM
xEASxxxxxxxxxxGxFVRAERPADLAxxxxxxxxxxxxxxxxxx
xxxxxxxxxxxxxxxxxxxxxxxxxxxxxxxxxxxxxxxRALGRx
xxxxDRRxxQxxxxxxxxxxxxxxxRxASxxxxxxxxxxxxxxxx
xxxxxxxxxxxxxxxxxxxxxxxxxxxxxxxxxxxxxx
SEQ ID NO. 83
xxxxxxxxxxxxxxxxxMPVxxGIGIGRGDPLRPAVTRTxRFSGP
EGFHxxPGALWLAAAAPLLATxLLLLxxRLAA
Representative amino acid
SEQuences for proteins
correlated with enhancing growth of rice
seedlings from specific Methylobacterium strains
are provided below as
SEQ ID NO: 84 through SEQ ID NO: 90.
The strain from which a representative
Sequence was obtained is referenced below.
LGP2022
SEQ ID NO: 84
MPTAIPIRPAPERCLSWPDTARLLILVARVRILDLEMHTVVRHGS
GFADDRLLHLMRRWLAQHEAISALLPGVAEPRHVAEVRAILQVPN
SRPEPEDRRAL*
LGP2021
SEQ ID NO: 85
MPQRRTIQVTEDGRMNLPADIRRVLGLNGAGRIVLMQDEDGIHLT
TAEDPLRRVRELAAPFRRGSGSVVDEFIAERRADSGED*
LGP2021
SEQ ID NO: 86
MPLDYALQITAIAFGLSVLGLGGAFIASRVYDRNTRRYDEAAQLH
KAD*
LGP2021
SEQ ID NO: 87
VQDGIQITCSVTEHVSLAYHENAVPVIREVVVENTSEQELSDVRV
RIESRPAVVQPLTLRIDRIPAGSNHHIELPDVRLDAALLAGFTEA
SRLELTVIVEDAAGERARHLEELRVLPPSHWGGGRSAPELLAAFV
RPNDPAVDVVLRDAATKLGEAGRETGLNGYTTAKKSRAWELABAI
WAAIADRRIAYVLPPASFERAGQKVRGPSDVLERKVGTCLDLSLL
YAACLEQAGLNPVLVLTVGHAFVGVWLQDDDFASATVDDMQLLRK
RRDLQDLVFVETTLLTPEPPATFKVATTOGGVQVEDEAPAALEIA
IDVRRCRRRGIRPMDLGDGKPTGIAPAPTIPLNQTLSAPPSFEEE
ARAPVDEAPETPVGRVERWKRKLLDLTLRNKLLNFKPGKGSVSLE
CASPGALEDGLAAGTEYRLKPLSDVLTGSDERSADLYARRHHDDG
RRSYLEAALARKEIYTTSTEADLDRRLLDLYRLARNGFEEGGANI
LFLAVGFLSWTKKEGEAAYRAPLLLVPVTLKRSSVRAGFKLALHD
DEVRINPTLLEMLREDFKLRMPELEGDLPRDGSGYDVDGIFRIVR
QHVKELRGWEVVPDVVLSAFSFTKYLMWKDLVDRAEVLKRNPVVR
HLIDTPKHSYGDGTPFPEPTRLDREHPPETVFAPLSADSSQLSAV
LAAAGGKDFVLFGPPGTGKSQTIGNMIAQCLAQGRTVLFVSQKTA
ALEVVQRRLQEIGLGDYCLEVHSTKAQKSAVLGQLRRAWHERSTP
SQGTWDAATSELASLREELNGLVNALHRRRENGLSAYEAFGRVIA
SGGEAPLVLTWPDHLAHNETTLANLRAACRELRPVLASVGSLVDH
PLQGVEATQWSPVWRDDMGAAIRAVEQTLGALRVSGQAFAEAIGL
PSLLATYAGTRGLVVLGNYLVRSEARCGAAFLADGAGDLRRAVAA
RERFQTTKVQLLGRLTGRYRPGILDQNLGALLAEWVAAQGANFLV
KGGKLKKVSAQVQPYAEGPLPPDLGPDLTGLIEVARHVKAGCLEE
LILARLGLPWSNPDCPASEFASAITWAEKVEQLLDILGPLSLGID
GLRDHLVHLVERQGRALADGGRIAQTYAAFAQDRARANEAMKALG
VLAGRPDPEEPLAAEADWIERSCTIARRLSSGLSRAQGWCAWQAA
AQSALKTGLAPLIDALEDGRIAPDRAEIAFEIAYARWWIDRVVSD
DPVLRRFLPARHEDAIQRFRAADARVTELSKQVVRSRLGGGIPGA
TAFGADPEWGTLSHELTKKTAHMPLRKLFGKMPTALTKLTPCVMM
SPLSIAQYLPPDKEPFDVVIFDEASQISPWDAIGALARAKQVVIV
GDPEQLPPTNVGDRGVDDIEDGSDVTDQESILDECLAANIPRRNL
DWHYRSRHESLIAFSNSRYYGGRLVTFPSPVTDDRAVRLTLVPDG
VYKRGSGRVNRPEARAVVADIVRRLRDPSFSEERRSLGVVTFNGE
QQRLIENLLDEQRRSYPELEPFFDRDRWHEPVFVKNLENVQGDER
DAIIFSVAVGPDQTGRPVSTVSSLNKDGGHRRLNVAITRARRELV
VFASMRPEQIDLGRTRARGVRDFKHFLEFAERGARALAEAFAPTG
GDVESPFEAAVMAGLEARGWTVHTQIGVSGFRIDLGIVHPDAPGR
YLAGVECDGATYHSSATARDRDRLREHVLTDLGWRIRRVWSTEWW
MDAEGALTKLDQRLIEDLEADRAKAAAAAAEAPRDVAVEPEAVEQ
EHDEPTGEPEVTPPVDTGPSEPANDLEPVTDLIPQRLYADQALPV
TPPAPKPEVYDDVRAYRIVDLNDLGRSVEPGRFYDASYQQALSAM
VDHVLAVEGPIYEELLIKRIARAHDIQRVGPLVREAIADRIDASV
ARTEDDGRPVLWPRGEEPRASYPHRPASAAIRSHTDTPMPELVGI
AMTLPSNASEAERARMIGQRLGLSRIEASARARFERASELARQAA
VA*
LGP2022
SEQ ID NO: 88
MSVVLYARVSTAEQTLEHQQTQAEAAGFVFDAVVADHGESGRKPL
RDRPEGRRLYDMLRTGDVLVVRWINRLGRSYEDVTGVMRELMORG
VIVRTIISNMTEDGATKDPMQRAIRDALIAFMAAAGEAELEATRE
AQKAGIEHARKQADQTAYRGRKPSYTRDQLTVISGMLGRGAGVSA
IAAETGLSRQTIYRVQADPVEAEAALARWA*
LGP2016
SEQ ID NO: 89
MLSLDDIAAAAAGEERDALWRSLVEDMEEAAGRRRGGRGLVQADR
PADLARALGRDRRVQPSRLARSAS*
LGP2022
SEQ ID NO: 90
MPVGIGIGRGDPLRPAVTRTARFSGPEGFHPGALWLAAASPLLAT
LLLLVRLAA*

### Example 8. Methylobacterium Inoculation Effect on Nitrogen Utilization in Rice

Methylobacterium isolates were tested for their ability to enhance shoot nitrogen content and/or concentration in rice. A randomized complete block design was used, with 12 treatments in each run; five Methylobacterium isolates and a control at two nitrogen levels. The untreated control sample (UTC) was Methylobacterium growth medium applied in the same amount as used for the Methylobacterium isolates. Each treatment level had an n of 10. All 10 blocks were grown in the same growth chamber and on the same shelf.

### Procedure:

### Media:

0.5× Murashige and Skoog MS medium with high or low nitrogen
   High nitrogen media—10400 uM
   Low nitrogen media—250 uM

### Pre-Planting:

Rice seeds were de-husked. Average 100 seed count is 2018 mg with approximately 21 g of husked rice per run.
Agar plates containing high or low nitrogen media were prepared.

### Planting:

Seeds were sterilized in ˜3% sodium hypochlorite+0.05% Tween 20.
Seeds were washed to remove bleach solution and placed on a sterile plate lid to begin drying.
Seeds were plated using a randomized complete block design with each complete block having similarly sized seeds.
Using sterile techniques 8 sterile seeds were evenly spaced in a horizontal line (˜ 40% above the bottom of the plate, using a pre-marked lid as a guide). Seeds were placed with the embryo toward the bottom of the plate and gently pushed into media.

### Inoculation:

Each Methylobacterium isolate or the culture medium control was applied as an 80 uL streak to the bottom portion of the plate (one isolate per plate) and spread by gently tilting the plate back and forth. A target concentration of 1×10⁶ CFU per seed was applied.
Plates were allowed to dry for at least one hour and placed in a randomized layout in a Percival growth chamber set to 25° C. and 16 hour days.
Seeds were allowed to grow undisturbed for 8 days.

### Harvest:

At 8 days after plating the plates were removed from the growth chambers, and the plants were measured as follows.
Plants that were not impeded from growing normally (by physical surroundings unrelated to presence of Methylobacterium) were removed from plates, and the number of seedlings for that plate was recorded.
Seedlings were scanned using WinRhizo and the images analyzed to determine root and shoot area for each plant.
Seedlings were rinsed to remove any remaining plate media and the shoots separated from the seedlings and dried in a drying oven for at least 3 days.
Dried shoots were combined for each treatment and the mass measured. The plant material was then ground to a powder to be used for nitrogen testing.
Nitrogen analysis was conducted on the powdered samples by Atlantic Microlab (Norcross, GA).

Results of the analyses are shown below. In all tables, pairwise results are presented separately for the High N and Low N treatments. Data was analyzed using Student's t-test and different letters indicate a significant difference between treatments at p<0.05.

TABLE 22
Exp 1
Shoot Area Measurements

| | |
|---|---|
| | Mean Shoot Area |
| Treatment | per Plant (cm²) |

22A Low Nitrogen Treatment

| | | |
|---|---|---|
| LGP2033 | A | 0.30 |
| UTC | A | 0.30 |
| LGP2009 | A | 0.29 |
| LGP2020 | A | 0.29 |
| LGP2022 | A | 0.28 |
| LGP2003 | A | 0.28 |

22B High Nitrogen Treatment

| | | |
|---|---|---|
| LGP2020 | A | 0.51 |
| LGP2033 | B | 0.42 |
| LGP2022 | BC | 0.40 |
| LGP2003 | BC | 0.40 |
| UTC | BC | 0.36 |
| LGP2009 | C | 0.34 |

TABLE 23
Exp 1
Root Area Measurements

| | |
|---|---|
| | Mean Root Area |
| Treatment | per Plant (cm²) |

23A Low Nitrogen Treatment

| | | |
|---|---|---|
| LGP2020 | A | 0.93 |
| LGP2022 | A | 0.88 |
| LGP2033 | AB | 0.85 |
| LGP2009 | B | 0.79 |
| LGP2003 | B | 0.77 |
| UTC | C | 0.64 |

23B High Nitrogen Treatment

| | | |
|---|---|---|
| LGP2020 | A | 0.99 |
| LGP2022 | B | 0.85 |
| LGP2033 | B | 0.83 |
| LGP2003 | C | 0.67 |
| LGP2009 | C | 0.62 |
| UTC | C | 0.59 |

TABLE 24
Exp 1
Shoot Nitrogen Concentration

| | |
|---|---|
| | Mean % Dry Wt |
| Treatment | Nitrogen |

24A Low Nitrogen Treatment

| | | |
|---|---|---|
| UTC | A | 2.73 |
| LGP2020 | B | 2.59 |
| LGP2022 | C | 2.48 |
| LGP2033 | C | 2.49 |
| LGP2009 | D | 2.35 |
| LGP2003 | D | 2.30 |

24B High Nitrogen Treatment

| | | |
|---|---|---|
| LGP2020 | A | 4.92 |
| LGP2022 | B | 4.38 |
| LGP2033 | C | 4.02 |
| UTC | D | 3.23 |
| LGP2009 | D | 3.27 |
| LGP2003 | D | 3.26 |

Significant and substantial shoot growth promotion was observed for some isolates at high nitrogen. Shoot growth promotion was not observed for the Methylobacterium treatments at low nitrogen, consistent with some literature reports which indicate that growth promotion effects from plant-beneficial microbes may not be observed when nutrient availability is too low. Root growth promotion was evident at both nitrogen levels, and Root/Shoot ratios are higher under low N than under high N. As expected, plants grown on high N media showed substantially greater shoot N concentration than those grown on low N media. Several Methylobacterium isolates demonstrated significantly enhanced shoot nitrogen concentration under high nitrogen growth conditions. Three isolates, LGP2020, LGP2022, and LGP2033, demonstrated the greatest enhancements of shoot growth, root growth, and shoot nitrogen concentration.

The above experiment was repeated using four of the same Methylobacterium isolates and one additional isolate. Results were similar to those observed in the first assay and are shown in the tables below. LGP2020 (NRRL B-67892), LGP2022 (NRRL B-68033), and LGP2033 (NRRL B-68068) again demonstrated enhancements of shoot growth, root growth, and shoot nitrogen concentration.

TABLE 25
Exp 2
Shoot Area Measurements

| | |
|---|---|
| | Mean Shoot Area |
| Treatment | per Plant (cm²) |

25A Low Nitrogen Treatment

| | | |
|---|---|---|
| LGP2022 | A | 0.18 |
| LGP2033 | A | 0.19 |
| LGP2020 | A | 0.17 |
| UTC | A | 0.19 |
| LGP2003 | A | 0.18 |
| LGP2019 | A | 0.18 |

25B High Nitrogen Treatment

| | | |
|---|---|---|
| LGP2022 | A | 0.30 |
| LGP2033 | AB | 0.30 |
| LGP2020 | AB | 0.29 |
| UTC | AB | 0.26 |
| LGP2003 | AB | 0.25 |
| LGP2019 | B | 0.25 |

TABLE 26
Exp 2
Root Area Measurements

| | |
|---|---|
| | Mean Root Area |
| Treatment | per Plant (cm²) |

26A Low Nitrogen Treatment

| | | |
|---|---|---|
| LGP2033 | AB | 0.57 |
| LGP2022 | AB | 0.53 |
| LGP2020 | A | 0.59 |
| LGP2019 | AB | 0.56 |
| LGP2003 | AB | 0.52 |
| UTC | B | 0.50 |

26B High Nitrogen Treatment

| | | |
|---|---|---|
| LGP2033 | A | 0.67 |
| LGP2022 | A | 0.66 |
| LGP2020 | A | 0.64 |
| LGP2019 | B | 0.54 |
| LGP2003 | B | 0.49 |
| UTC | B | 0.47 |

TABLE 27
Exp 2
Shoot Nitrogen Concentration

| | |
|---|---|
| | Mean % Dry Wt |
| Treatment | Nitrogen |

27A Low Nitrogen Treatment

| | | |
|---|---|---|
| LGP2020 | AB | 2.36 |
| LGP2022 | AB | 2.30 |
| LGP2033 | AB | 2.38 |
| UTC | A | 2.51 |
| LGP2003 | B | 2.25 |
| LGP2019 | B | 2.21 |

27B High Nitrogen Treatment

| | | |
|---|---|---|
| LGP2020 | A | 4.28 |
| LGP2022 | A | 4.06 |
| LGP2033 | B | 3.68 |
| UTC | BC | 3.45 |
| LGP2003 | C | 3.37 |
| LGP2019 | C | 3.23 |

Percent difference between Methylobacterium treatments and UTC at high and low N for 3 different variables: projected root area, projected shoot area, and foliar nitrogen concentration are shown for each experiment. Bold italics are used to denote a statistically significant difference from UTC at p<0.05 using Student's t-test.

TABLE 28
Percent Differences

| | | | | | | % N | % N |
|---|---|---|---|---|---|---|---|
| N | | % Root | % Root | % Shoot | % Shoot | Enhancement | Enhancement |
| Level | Treatment | GP Exp 1 | GP Exp 2 | GP Exp 1 | GP Exp 2 | Exp 1 | Exp 2 |
| High | LGP2003 | +15.1% | +2.8% | +10.6% | −1.7% | −0.8% | −2.2% |
| N | LGP2020 | +68.5% | +35.0% | +42.0% | +14.0% | +49.7% | +23.9% |
| | LGP2033 | +41.6% | +42.2% | +16.2% | +15.5% | +22.4% | +6.8% |
| | LGP2022 | +45.4% | +40.1% | +10.8% | +15.8% | +33.3% | +17.7% |
| Low | LGP2003 | +19.4% | +4.5% | −8.9% | −8.6% | −15.8% | −10.2% |
| N | LGP2020 | +43.5% | +18.3% | −3.2% | −11.5% | −5.3% | −6.1% |
| | LGP2033 | +31.8% | +13.8% | +0.7% | −2.5% | −9.1% | −5.0% |
| | LGP2022 | +37.0% | +6.1% | −8.6% | −8.5% | −9.0% | −8.3% |

### Example 9. Evaluation of Optimal Nitrogen Dose for Testing Methylobacterium Effect

The high nitrogen dose in the experiments described above is the amount in 0.5× MS media, a general plant growth medium, and provides a luxury amount of nitrogen for plant growth. To evaluate plant response to Methylobacterium treatment under various reduced nitrogen levels, including a nitrogen level that approximates the amount of nitrogen in a field treated with a 25-30% reduction of optimal nitrogen level, two low nitrogen dose experiments were conducted.

Experiment 3 was conducted as described in Example 8, except that the nitrogen doses used for evaluation of effect of Methylobacterium treatment on plant growth were: 5200 uM nitrogen (70% of rice optimal nitrogen level), 7280 uM nitrogen (rice optimal nitrogen level), and 10400 uM nitrogen (rice luxury nitrogen level). Results are shown in Tables 29-31 below. Data was analyzed using Student's t-test, and different letters indicate a significant difference between treatments at p<0.05.

TABLE 29
Exp 3
Shoot Area Measurements

| | 5200 μM N | | 7280 μM N | | 10400 μM N | |
|---|---|---|---|---|---|---|
| | Treatment | | Treatment | | Treatment | |
| | Mean Shoot | | Mean Shoot | | Mean Shoot | |
| | Area per Plant | | Area per Plant | | Area per Plant | |
| Treatment | (cm²) | | (cm²) | | (cm²) | |
| LGP2020 | A | 0.41 | A | 0.36 | A | 0.41 |
| LGP2033 | B | 0.33 | A | 0.34 | B | 0.34 |
| Control | C | 0.28 | B | 0.25 | BC | 0.30 |
| LGP2019 | C | 0.27 | B | 0.28 | C | 0.28 |

TABLE 30
Exp 3
Root Area Measurements

| | 5200 μM N | | 7280 μM N | | 10400 μM N | |
|---|---|---|---|---|---|---|
| | Treatment | | Treatment | | Treatment | |
| | Mean Root | | Mean Root | | Mean Root | |
| | Area per Plant | | Area per Plant | | Area per Plant | |
| Treatment | (cm²) | | (cm²) | | (cm²) | |
| LGP2020 | A | 0.82 | A | 0.78 | A | 0.79 |
| LGP2033 | B | 0.70 | A | 0.77 | B | 0.71 |
| LGP2019 | B | 0.62 | B | 0.64 | C | 0.57 |
| Control | C | 0.47 | C | 0.45 | D | 0.49 |

TABLE 31
Exp 3
Shoot Nitrogen Concentration

| | 5200 μM N | | 7280 μM N | | 10400 μM N | |
|---|---|---|---|---|---|---|
| | Treatment Mean | | Treatment Mean | | Treatment Mean | |
| | % Dry | | % Dry | | % Dry | |
| Treatment | Wt Nitrogen | | Wt Nitrogen | | Wt Nitrogen | |
| LGP2020 | A | 4.70 | A | 4.40 | A | 4.61 |
| LGP2033 | B | 3.77 | B | 4.02 | B | 3.96 |
| LGP2019 | C | 3.14 | C | 3.42 | C | 3.41 |
| Control | C | 3.13 | C | 3.22 | C | 3.34 |

Experiment 3 was conducted as described in Example 8, except that the nitrogen doses used for evaluation of effect of Methylobacterium treatment on plant growth were: 1560 uM nitrogen (20% of rice optimal nitrogen level), 2600 uM nitrogen (35% of rice optimal nitrogen level), and 5200 uM nitrogen. (70%0 of rice optimal nitrogen level). Results are shown in Tables 32-34 below.

TABLE 32
Exp 4
Shoot Area Measurements

| | 1560 μM N | | 2600 μM N | | 5200 μM N | |
|---|---|---|---|---|---|---|
| | Treatment | | Treatment | | Treatment | |
| | Mean Shoot | | Mean Shoot | | Mean Shoot | |
| | Area per Plant | | Area per Plant | | Area per Plant | |
| Treatment | (cm²) | | (cm²) | | (cm²) | |
| LGP2020 | A | 0.28 | A | 0.32 | A | 0.38 |
| LGP2017 | A | 0.27 | AB | 0.28 | AB | 0.31 |
| LGP2019 | AB | 0.26 | B | 0.26 | B | 0.26 |
| Control | B | 0.23 | C | 0.22 | B | 0.25 |

TABLE 33
Exp 4
Root Area Measurements

| | 1560 μM N | | 2600 μM N | | 5200 μM N | |
|---|---|---|---|---|---|---|
| | Treatment | | Treatment | | Treatment | |
| | Mean Root | | Mean Root | | Mean Root | |
| | Area per Plant | | Area per Plant | | Area per Plant | |
| Treatment | (cm²) | | (cm²) | | (cm²) | |
| LGP2020 | A | 0.75 | A | 0.73 | A | 0.71 |
| LGP2017 | AB | 0.72 | B | 0.65 | AB | 0.66 |
| LGP2019 | B | 0.65 | B | 0.63 | B | 0.61 |
| Control | C | 0.45 | C | 0.44 | C | 0.45 |

TABLE 34
Exp 4
Shoot Nitrogen Concentration

| | 1560 μM N | | 2600 μM N | | 5200 μM N | |
|---|---|---|---|---|---|---|
| | Treatment Mean | | Treatment Mean | | Treatment Mean | |
| | % Dry | | % Dry | | % Dry | |
| Treatment | Wt Nitrogen | | Wt Nitrogen | | Wt Nitrogen | |
| LGP2020 | A | 3.03 | A | 3.65 | A | 4.67 |
| LGP2017 | A | 3.00 | B | 3.51 | B | 4.22 |
| LGP2019 | AB | 2.86 | C | 3.30 | C | 3.25 |
| Control | B | 2.73 | D | 2.90 | C | 3.15 |

Results of Experiments 3 and 4 again demonstrate significant and substantial shoot and root growth promotion and increased levels of shoot nitrogen levels resulting from treatment with Methylobacterium isolates. Shoot area correlated closely to nitrogen levels measured in shoots. Although root area measurements were not observed to be in proportion to increased nitrogen uptake as measured in shoots, additional observations noted that numbers of root tips were increased in line with enhanced nitrogen uptake as measured in shoot nitrogen concentration.

Experiments to identify additional Methylobacterium strains that can enhance plant growth and development under reduced nitrogen levels will be conducted using a 5200 μM nitrogen treatment, representing 70% of the optimal N level for rice, or a 30% reduction in nitrogen fertilizer application for rice cultivation.

### Example 10. Methylobacterium Treated Corn Plants Grown Under Reduced Nitrogen

Corn seeds treated Methylobacterium were grown in a large-scale field trial under reduced nitrogen conditions to determine effects on foliar nitrogen levels and corn yield. The trial was conducted at nine locations using a randomized complete block design at each location with 3 reps per location. Methylobacterium LGP2019 (NRRL B-67743) was applied in-furrow at planting with starter fertilizer applied at 150 lbs N per acre, a 25% reduction of the standard nitrogen fertilizer rates at the midwestern US locations. The Methylobacterium was applied at a rate of approximately 1×10⁶ CFU per seed to corn hybrid Croplan CP4488SS/RIB, a 104-day hybrid with a high response to nitrogen. Some data points were culled from the final dataset due to environmental stress or as statistical outliers, including removal of all data from one high stress location.

Foliar tissue from the ear leaf at the R2-R4 developmental stage was sampled for nitrogen, phosphorus, and potassium nutrient concentrations. Corn seed was harvested at maturity and seed yield determined. Results are presented in the Tables below.

TABLE 35
Tissue nutrient concentrations

| | Tissue N | Tissue P | Tissue K |
|---|---|---|---|
| | concentration | concentration | concentration |
| Treatment | (% by mass) | (% by mass) | (% by mass) |
| LGP2019 | 2.76 | 0.35 | 1.77 |
| UTC | 2.81 | 0.36 | 1.83 |

TABLE 36
Yield

| | UTC | LGP2019 |
|---|---|---|
| Location | Yield (Bu/A) | Yield (Bu/A) |
| Steuben, WI (1) | 176.2 | 193.7 |
| Steuben, WI (2) | 174.0 | 184.1 |
| Lime Springs, IA | 174.5 | 180.3 |
| Fairbank, IA | 171.5 | 175.1 |
| Waverly, IL (1) | 207.9 | 209.8 |
| Waverly, IL (2) | 207.9 | 206.6 |
| New Hampton, IA | 180.6 | 179.6 |
| South Park, NE | 164.3 | 157.8 |
| Total | 179.9 | 184.6* |

*indicates significant yield difference between UTC and LG2019 at p < 0.1.

Nutrient content of foliar tissue collected at the R2-R4 developmental stage was not significantly different in the treated plants in comparison to an untreated control. Harvested seed yield was significantly increased over the untreated control plant yields when analyzed over all 8 locations, demonstrating that Methylobacterium LGP2019 enhances nitrogen uptake under reduced nitrogen growth conditions and provides for increased seed yield.

To further analyze the effect of treatment of corn seeds with Methylobacteirum LGP2019, a second field trial was conducted using standard nitrogen application rates and foliar nutrient contents analyzed at two timepoints. LGP2019 was applied in furrow at planting at a rate of approximately 1×10⁶ CFU per seed to 12 corn hybrids in a non-replicated strip trial. Each strip contained a biostimulant and hybrid combination and was 4 rows wide and ⅛ to ¼ of a mile long in a commercial field in Pittsfield, IL. Aboveground tissue samples were taken to assess foliar nutrient concentrations at V2-V3 (May 27) and at tasseling (July 8). Two of the 12 hybrids planted were selected for tissue sampling and were aggregated for analysis: Lewis 15 DP 899 VT2PRIB and AgriGold A6659 VT2. One data point was generated per sampling period.

Results are presented in Tables 36 and 37 below. Seed yield was not significantly different from the untreated control in this trial that used standard nitrogen fertilizer rates.

TABLE 37
Seed Yield

| Treatment | Yield (Bu/A) |
|---|---|
| UTC | 243.7 |
| LGP2019 | 242.6 |

TABLE 38
Tissue nutrient concentrations

| | |
|---|---|
| V2-V3 Stage | VT-R1 Stage |

| Nutrient | UTC | LGP2019 | UTC | LGP2019 |
|---|---|---|---|---|
| N_pct | 3.34 | 4.37 | 3.83 | 4.23 |
| P_pct | 0.24 | 0.227 | 0.367 | 0.393 |
| K_pct | 3.89 | 4.05 | 2.09 | 2.31 |
| Ca_pct | 1.19 | 1.07 | 0.55 | 0.63 |
| Mg_pct | 0.233 | 0.207 | 0.243 | 0.203 |
| S_pct | 0.278 | 0.309 | 0.253 | 0.3 |
| B_ppm | 7.6 | 7.5 | 6.5 | 8.3 |
| Fe_ppm | 520 | 514 | 113 | 127 |
| Mn_ppm | 113 | 112 | 61.4 | 73.6 |
| Cu_ppm | 7.3 | 8.2 | 13.6 | 14.6 |
| Zn_ppm | 22.4 | 25.8 | 26.9 | 31.2 |

Increased levels of nitrogen, potassium, sulfur, copper, and zinc were detected in V2-V3 and VT-R1 stage tissue samples. In addition, increased levels of phosphorus, boron, iron, and manganese were detected in stage VT-R1 stage corn tissue.

### Example 11. Increases in Rice Yield by Application of Methylobacterium

Rice field trials were conducted at three locations, all near Humphrey, AR, for the purpose of evaluating the effects of three Methylobacterium isolates applied as a seed treatment. Treatments included each Methylobacterium isolate and an untreated control applied to rice seeds with and without a base treatment of insecticide only (active ingredient Clothiandin). The trial was conducted using a Randomized Complete Block Design (RCBD) with 4 reps per location. LGP2016 (NRRL B-67341), LGP2019 (NRRL B-67743), and LGP2017 (NRRL B-67741) were applied to rice seeds at a target concentration of 10⁶ CFU/seed.

The Methylobacterium isolates increased yield in rice field trials as compared to the untreated control both with and without insecticide treatment as shown in the Table below.

TABLE 39
Mean yield (Bu/A) Increase over control
and percent increase shown
(Bold italics indicates a significant difference
at p < 0.05 using Fisher's LSD test.)

| Treatment | UTC | LGP2016 | | LGP2019 | | LGP2017 | |
|---|---|---|---|---|---|---|---|
| Without | 143.8 | 150.1 | +6.3 | 156.2 | +12.4 | 152.4 | +8.6 |
| insecticide | | | (4.3%) | | (8.6%) | | (6.0%) |
| treatment | | | | | | | |
| With | 151.8 | 164.3 | +12.5 | 155.4 | +3.6 | 158.2 | +6.4 |
| insecticide | | | (8.2%) | | (2.4%) | | (4.2%) |
| treatment | | | | | | | |

Also provided herein are methods of improving growth and yield of rice plants by treating rice plants, plant parts, or seeds with one or more Methylobacterium isolates. In some embodiments, harvested seed yield and/or nutrient content of rice plants is improved. In some embodiments, rice seeds are treated and such treatment provides for increased rice seed yield. In some embodiments, the Methylobacterium isolate is selected from the group consisting of LGP2016 (NRRL B-67341), LGP2017 (NRRL B-67741), LGP2019 (NRRL B-67743), and variants of these isolates. Rice plants, plant parts, or seeds coated with Methylobacterium isolates and/or compositions are also provided herein. In certain embodiments, the Methylobacterium has chromosomal genomic DNA having at least 99%, 99.9, 99.8, 99.7, 99.6%, or 99.5% sequence identity to chromosomal genomic DNA of LGP2016, LGP2017, or LGP2019. In certain embodiments, the Methylobacterium has genomic DNA comprising one or more polynucleotide marker fragments of at least 50, 60, 100, 120, 180, 200, 240, or 300 nucleotides of SEQ ID NOS: 37-39 or SEQ ID NOS: 25-27.

### Example 12. Procedure to Test Hits Identified from Methylobacterium Inoculation Effect on Promotion of Early Rice Growth for Methylobacterium Inoculation Effect on Nitrogen Utilization in Rice

Additional Methylobacterium strains, including Methylobacterium strains that caused increased root length during early rice growth from Example 7, are tested for Methylobacterium inoculation effect on nitrogen utilization in rice.

The experiment is conducted using the method as described in Example 8, except replacing the high and low nitrogen conditions with using 5200 uM nitrogen (70% of rice optimal nitrogen level) as described in Example 9. Data can be analyzed using Student's t-test to determine significant differences between strains at p<0.05 to determine strains that have increased nitrogen uptake compared to untreated control samples.

Results shown in Table 40 below provide percent differences in foliar N concentration in treated rice plants compared to N levels in untreated seedlings. Foliar tissue was harvested, dried, and assayed for nitrogen concentration via elemental combustion analysis.

TABLE 40

| | Percent difference from | |
|---|---|---|
| | Untreated in Foliar | Number |
| Methylobacterium | N concentration | of times |
| Strain | (% by mass) | tested |
| LGP2020 | +45.2% | 9 |
| LGP2023 | +47.6% | 1 |
| LGP2031 | +38.2% | 3 |
| LGP2034 | +43.9% | 1 |
| LGP2029 | +35.7% | 3 |
| LGP2021 | +41.0% | 1 |
| LGP2167 | +40.5% | 1 |
| LGP2030 | +32.0% | 3 |
| LGP2002 | +42.8% | 1 |
| LGP2018 | +37.5% | 1 |
| LGP2001 | +29.2% | 1 |
| LGP2015 | +27.9% | 1 |
| LGP2188 | +3.0% | 1 |
| LGP2189 | −4.8% | 1 |
| LGP2005 | −4.9% | 1 |
| LGP2004 | −4.7% | 1 |

### Example 13. Analysis of Yield and Nitrogen Use Efficiency of Methylobacterium Treated Corn and Wheat Plants

Wheat field trials were conducted using a Randomized Complete Block Design (RCBD) with 5 treatments replicated 5 times. Treatments include 0% N, 100% N only (100%=180 lbs/A), 85% N+Methylobacterium NRRL B-67743 (LGP2019), 70% N+Methylobacterium NRRL B-67743 (LGP2019), and 70% N only. Methylobacterium treatments are applied to corn or wheat seeds at a target concentration of 10⁶ CFU/seed. Corn seeds were treated by in furrow application. Wheat seedlings were treated at transplant to simulate in furrow application. Data were collected and statistically analyzed to evaluate effects of the Methylobacterium isolates on yield and nitrogen use efficiency including soil N, P, and K levels prior to planting, plant tissue N, P, and K concentration and content (uptake), calculated NUE, root architecture, total plant biomass (shoots and fruits), and grain yield. The results of these trials revealed that application of 85% N+Methylobacterium NRRL B-67743 (LGP2019) or 70% N+Methylobacterium NRRL B-67743 (LGP2019) provided for a dry biomass and N content that was statistically the same as the 100% N treatment.

Additional wheat and corn field trials are conducted using a Randomized Complete Block Design (RCBD) with 5 treatments replicated 5 times. Treatments include 0% N, 100% N only (100%=180 lbs/A), 85% N+Methylobacterium NRRL B-67743 (LGP2019) or Methylobacterium NRRL B-67892 (LGP2020), 70% N+Methylobacterium NRRL B-67743 (LGP2019) or Methylobacterium NRRL B-67892 (LGP2020), and 70% N only. The two Methylobacteirum isolates are tested in separate, adjacent trials. Methylobacterium treatments are applied to corn or wheat seeds at a target concentration of 10⁶ CFU/seed. Corn seeds are treated by in furrow application. Wheat seedlings are treated at transplant to simulate in furrow application. Data are collected and statistically analyzed to evaluate effects of the Methylobacterium isolates on yield and nitrogen use efficiency including soil N, P, and K levels prior to planting, plant tissue N, P, and K concentration and content (uptake), calculated NUE, root architecture, total plant biomass (shoots and fruits), and grain yield.

### Example 14. Methylobacterium Treatment of Herbs

Effects of Methylobacterium treatment of Pennisetum, basil, French tarragon, rosemary, and oregano were evaluated. Direct seeded plants, transplants, or plants produced by vegetative propagation were treated by applying Methylobacterium as a drench at seedling, transplanting, or at sticking (for plants produced by vegetative propagation). Improvements in flowering, bushiness, leaf area, rooting, root length, and biomass were observed as shown in the table below.

TABLE 41

| Herb | Methylobacterium treatment | Observations |
|---|---|---|
| PENNISETUM | i) LGP2009 (NRRL B-50938) | 2X increase in flowering compared |
| | ii) LGP2015 (NRRL B-67340) | to controls at 12 weeks after |
| | Treatments applied at transplant. | transplanting; visible increase |
| | | in plant bushiness |
| BASIL | i) LGP2009 (NRRL B-50938) | 30% increase in leaf area at 28 |
| | ii) Combination of LGP2002 | days after planting vs. control |
| | (NRRL B-50931) and LGP2015 | |
| | (NRRL B-67340) | |
| | Treatments applied at seeding. | |
| FRENCH | LGP2001 (NRRL B-50930) | Enhanced rooting vs. control |
| TARRAGON | Treatment applied at vegetative | |
| | propagation. | |
| ROSEMARY | LGP2002 (NRRL B-50931) | 30% increase in dry biomass, 2X |
| | Treatment applied at vegetative | increase in fine root length at 28 |
| | propagation. | days after planting vs. control |
| OREGANO | Combination of LGP2009 | 2X increase in total root length at |
| | (NRRL B-50938) with | 14 days after planting vs. control |
| | LGP2001 (NRRL B-50930) | |
| | Treatment applied at vegetative | |
| | propagation. | |

### Example 15. Additional Methylobacterium Strains Tested for Enhanced Nitrogen Utilization

Additional Methylobacterium strains are tested for Methylobacterium inoculation effect on nitrogen utilization in rice. The experiment is conducted using the method as described in Example 12. Data is analyzed using Student's t-test to determine significant differences between strains at p<0.05 to determine strains that have increased nitrogen uptake compared to untreated control samples. Results shown in Table 43 below provide percent differences in foliar N concentration in treated plants compared to N levels in untreated seedlings. Foliar tissue was harvested, dried, and assayed for nitrogen concentration via elemental combustion analysis.

TABLE 42

| Methylobacterium | Percent difference from Untreated in |
|---|---|
| Strain | Foliar N concentration (% by mass) |
| LGP2032 | +30.0% |
| LGP2024 | +31.3% |
| NLS0681 | +27.2% |
| NLS0594 | +24.2% |
| NLS0479 | +43.2% |
| NLS1310 | +44.2% |
| NLS0612 | +38.1% |
| NLS1312 | +36.5% |
| NLS0473 | +32.6% |
| NLS0706 | +34.5% |
| NLS0725 | +34.9% |
| NLS0159 | +5.1% |
| NLS0229 | −1.5% |

Additional Methylobacterium strains that provide for enhanced nitrogen utilization of treated plants are identified using rice plate assays as described above and greenhouse pot assays. A nitrogen content of 7280 uM (7000 of rice luxury N content) is used in all treatments. Treatments are compared to untreated control plates or untreated greenhouse pots that also received 7280 uM nitrogen. Results are shown in Table 43 below. Results demonstrate varying levels of improvement in shoot N concentration and biomass, and root length in plate assays for some tested strains. Greenhouse pot assays demonstrate increases in shoot and reproductive tissue biomass resulting from treatment several Methylobacterium strains, including NLS0665 (NRRL B-68194), NLS0754, NLS0693, NLS0591 (NRRL 1B-68215), LGP2020 and LGP219. Increased reproductive tissue biomass is an indication of a strain's ability to improve rice yield, and substantial increases were observed from treatment with LGP2020, LGP2019, NLS0754 and NLS0665 (NRRL B-68194).

TABLE 43

| | Plate assay | | | | |
|---|---|---|---|---|---|
| | Average | | | | |
| | Shoot N | | | GH assay | |
| | concentration | | | Average Shoot | Average |
| | (% by mass) | Average Shoot | Average Root | biomass | Reproductive |
| | percent | Projected Area | Length per | percent | tissue biomass |
| | difference | per Plant percent | Plant percent | difference | percent |
| | from 70% N | difference from | difference from | from 70% | difference from |
| Strain | control | 70% N control | 70% N control | control | 70% control |
| NLS0665 | 43.1 | 37.3 | 58.1 | 6.0 | 9.8 |
| LGP2018 | 37.5 | 12.9 | 71.7 | | |
| NLS0754 | 40.8 | 40.8 | 58.9 | 11.0 | 10.8 |
| NLS0049 | 40.6 | 42.8 | 66.5 | | |
| NLS0693 | 32.9 | 27.0 | 46.0 | 2.9 | 7.3 |
| NLS0591 | 34.3 | 30.9 | 52.3 | 6.6 | 6.5 |
| NLS0672 | 31.4 | 26.6 | 46.6 | | |
| NLS0729 | 34.1 | 33.0 | 39.2 | −1.9 | 4.2 |
| NLS0439 | 37.3 | 40.7 | 54.0 | | |
| LGP2017 | 28.4 | 28.3 | 42.5 | | |
| NLS1310 | 44.2 | 24.9 | 92.3 | | |
| NLS1312 | 36.5 | 21.9 | 96.7 | | |
| LGP2020 | 41.8 | 34.4 | 81.8 | 10.8 | 15.3 |
| LGP2019 | −2.1 | 7.0 | 19.5 | 1.1 | 12.9 |
| NLS0612 | 38.1 | 40.9 | 112.5 | | |
| NLS0706 | 34.5 | 37.4 | 80.4 | | |
| NLS0725 | 34.9 | 25.4 | 91.7 | | |

### Example 16. Genetic Sequences

16S RNA sequences are disclosed as SEQ ID NOS:91-120 for Methylobacterium strains for enhanced nitrogen utilization: LGP2002 (NRRL B-50931), LGP2001 (NRRL B-50930), LGP2015 (NRRL B-67340), LGP2021 (NRRL B-68032), LGP2020 (NRRL B-67892), LGP2017 (NRRL B-67741), LGP2018 (NRRL B-67742), LGP2029 (NRRL B-68065), LGP2030 (NRRL B-68066), LGP2019 (NRRL B-67743), LGP2031 (NRRL B-68067), LGP2016 (NRRL B-67341), LGP2033 (NRRL B-68068), LGP2034 (NRRL B-68069), LGP2022 (NRRL B-68033), LGP2023 (NRRL B-68034), LGP2167 (NRRL B-67927), NLS1310, NLS0612 (NRRL B-68237), NLS1312NLS0706 (NRRL B-68238), NLS0725 (NRRL B-68239), NLS0665 (NRRL B-68194), NLS0729 (NRRL B-68195), NLS0672 (NRRL B-68196), NLS0754 (NRRL B-68197), NLS0591 (NRRL B-68215), NLS0439 (NRRL B-68216), NLS0049 (NRRL B-68236), and NLS0693 (NRRL B-67926.

| SEQ | |
|---|---|
| ID NO | Isolate NO |
| 91 | LGP2002 |
| 92 | LGP2001 |
| 93 | LGP2015 |
| 94 | LGP2021 |
| 95 | LGP2020 |
| 96 | LGP2017 |
| 97 | LGP2018 |
| 98 | LGP2029 |
| 99 | LGP2030 |
| 100 | LGP2019 |
| 101 | LGP2031 |
| 102 | LGP2016 |
| 103 | LGP2033 |
| 104 | LGP2034 |
| 105 | LGP2022 |
| 106 | LGP2023 |
| 107 | LGP2167 |
| 108 | NLS1310 |
| 109 | NLS0612 |
| 110 | NLS1312 |
| 111 | NLS0706 |
| 112 | NLS0725 |
| 113 | NLS0665 |
| 114 | NLS0729 |
| 115 | NLS0672 |
| 116 | NLS0754 |
| 117 | NLS0591 |
| 118 | NLS0439 |
| 119 | NLS0049 |
| 120 | NLS0693 |

Genomic sequences that can be used to identity and distinguish Methylobacterium strains identified herein or variants and derivatives thereof are identified by an exact k-mer analysis of whole genome sequences of over 5000 public and proprietary Methylobacterium isolates. Sequences are provided below as SEQ ID NOS:121-131.

| SEQ | |
|---|---|
| ID NO | Isolate NO |
| 121 | NLS0049 |
| 122 | NLS0439 |
| 123 | NLS0591 |
| 124 | NLS0612 |
| 125 | NLS0665 |
| 126 | NLS0672 |
| 127 | NLS0706 |
| 128 | NLS0725 |
| 129 | NLS0754 |
| 130 | NLS1310 |
| 131 | NLS1312 |

### REFERENCES

Green, P. N. 2005. Methylobacterium. In Brenner, D. J., N. R. Krieg, and J. T. Staley (eds.). “Bergey's Manual of Systematic Bacteriology. Volume two, The Proteobacteria. Part C, The alpha-, beta-, delta-, and epsilonproteobacteria.” Second edition. Springer, New York. Pages 567-571.
Green, P. N. and Ardley, J. K. 2018. Review of the genus Methylobacterium and closely related organisms: a proposal that some Methylobacterium species be reclassified into a new genus, Methylorubrum gen. nov. Int J Syst Evol Microbiol. 2018 September; 68(9):2727-2748. doi: 10.1099/ijsem.0.002856.
Konstantinidis K. T., Ramette A., Tiedje J. M. (2006;). The bacterial species definition in the genomic era. Philos Trans R Soc Lond B Biol Sci 361: 1929-1940.
Lidstrom, M. E. 2006. Aerobic methylotrophic prokaryotes. In Dworkin, M., S. Falkow, E. Rosenberg, K.-H. Schleifer, and E. Stackebrandt (eds.). “The Prokaryotes. A Handbook on the Biology of Bacteria. Volume 2. Ecophysiology and biochemistry.” Third edition. Springer, New York. Pages 618-634.
Sy, A., Giraud, E., Jourand, P., Garcia, N., Willems, A., De Lajudie, P., Prin, Y., Neyra, M., Gillis, M., Boivin-Masson, C., and Dreyfus, B. 2001. Methylotrophic Methylobacterium Bacteria Nodulate and Fix Nitrogen in Symbiosis with Legumes. Jour. Bacteriol. 183(1):214-220.

The breadth and scope of the present disclosure should not be limited by any of the above-described embodiments, but should be defined only in accordance with the following claims and their equivalents.

What is claimed is:

1. A method for enhancing plant production that comprises:
(a) applying a composition to a plant, plant part, or seed, wherein the composition comprises at least one Methylobacterium selected from the group consisting of NLS0665 (NRRL B-68194), NLS0754 (NRRL B-68197), NLS0672 (NRRL B-68196), NLS0693 (NRRL B-67926), NLS0729 (NRRL B-68195), NLS0049 (NRRL B-68236), NLS0591 (NRRL B-68215), NLS0439 (NRRL B-68216), NLS1310, NLS1312, NLS0612 (NRRL B-68237), NLS0706 (NRRL B-68238), NLS0725 (NRRL B-68239), LGP2020 (NRRL B-67892), LGP2021 (NRRL B-68032), LGP2022 (NRRL B-68033), LGP2023 (NRRL B-68034), LGP2029 (NRRL B-68065), LGP2030 (NRRL B-68066), LGP2031 (NRRL B-68067), LGP2033 (NRRL B-68068), LGP2034 (NRRL B-68069), and LGP2167 (NRRL B-67927), and variants thereof, and,
(b) growing the plant to at least a two leaf stage, thereby enhancing at least one plant trait selected from the group consisting of early plant growth, propagation/transplant vigor, nutrient uptake, stand establishment, stress tolerance, and nutrient utilization efficiency;
wherein said trait is enhanced in comparison to an untreated control plant that had not received an application of the composition or in comparison to a control plant grown from an untreated seed that had not received an application of the composition.

2. The method of claim 1, wherein the Methylobacterium is selected from the group consisting of NLS0665 (NRRL B-68194), NLS0754 (NRRL B-68197), NLS0672 (NRRL B-68196), NLS0729 (NRRL B-68195), NLS0049 (NRRL B-68236), NLS0591 (NRRL B-68215), and NLS0439 (NRRL B-68216).

3. The method of claim 1 or 2, wherein the composition is applied to a seed.

4. The method of any one of claims 1 to 3, wherein said plant is a leafy green plant.

5. The method of claim 4, wherein said leafy green plant is selected from the group consisting of spinach, lettuce, beets, swiss chard, watercress, kale, collards, escarole, arugula, endive, bok choy, and turnips.

6. The method of claim 4 or 5, wherein said leafy green plant is cultivated for production and selected from a group consisting of microgreens, herbs, and combinations thereof.

7. The method of any one of claims 1 to 3, wherein said plant is an agricultural row plant.

8. The method of any one of claims 1 to 3, wherein said plant is rice.

9. The method of any one of claims 1 to 8, wherein said composition further comprises at least one additional component selected from the group consisting of an additional active ingredient, an agriculturally acceptable adjuvant, and an agriculturally acceptable excipient.

10. The method of claim 9 wherein said composition comprises an agriculturally acceptable adjuvant selected from the group consisting of polyvinyl acetates, polyvinyl acetate copolymers, hydrolyzed polyvinyl acetates, polyvinylpyrrolidone-vinyl acetate copolymer, polyvinyl alcohols, polyvinyl alcohol copolymers, polyvinyl methyl ether, polyvinyl methyl ether-maleic anhydride copolymer, waxes, latex polymers, celluloses including ethylcelluloses and methylcelluloses, hydroxy methylcelluloses, hydroxypropylcellulose, hydroxymethylpropylcelluloses, polyvinyl pyrrolidones, alginates, dextrins, malto-dextrins, polysaccharides, fats, oils, proteins, karaya gum, jaguar gum, tragacanth gum, polysaccharide gums, mucilage, gum arabics, shellacs, vinylidene chloride polymers and copolymers, soybean-based protein polymers and copolymers, lignosulfonates, acrylic copolymers, starches, polyvinylacrylates, zeins, gelatin, carboxymethylcellulose, chitosan, polyethylene oxide, acrylamide polymers and copolymers, polyhydroxyethyl acrylate, methylacrylamide monomers, alginate, ethylcellulose, polychloroprene and syrups or mixtures thereof.

11. The method of claim 9 wherein said composition comprises an agriculturally acceptable excipient selected from the group consisting of woodflours, clays, activated carbon, diatomaceous earth, fine-grain inorganic solids and calcium carbonate.

12. A composition comprising a fermentation product comprising a Methylobacterium strain, wherein said fermentation product is essentially free of contaminating microorganisms, and wherein the Methylobacterium strain is selected from the group consisting of NLS0665 (NRRL B-68194), NLS0754 (NRRL B-68197), NLS0672 (NRRL B-68196), NLS0729 (NRRL B-68195), NLS0049 (NRRL B-68236), NLS0591 (NRRL B-68215), NLS0439 (NRRL B-68216), NLS1310, NLS1312, NLS0612 (NRRL B-68237), NLS0706 (NRRL B-68238), NLS0725 (NRRL B-68239), and variants thereof.

13. The composition of claim 12, wherein the Methylobacterium strain is selected from the group consisting of NLS0665 (NRRL B-68194), NLS0754 (NRRL B-68197), NLS0672 (NRRL B-68196), NLS0729 (NRRL B-68195), NLS0049 (NRRL B-68236), NLS0591 (NRRL B-68215), and NLS0439 (NRRL B-68216).

14. The composition of claim 12 or 13, wherein said composition further comprises at least one additional component selected from the group consisting of an additional active ingredient, an agriculturally acceptable adjuvant, and an agriculturally acceptable excipient.

15. A plant, plant part, or seed at least partially coated with the composition of any one of claims 12 to 14.

16. A method of enhancing growth and/or yield of a plant, wherein said method comprises treating said plant or soil where said plant is growing or will be grown, with a Methylobacterium isolate that enhances uptake and/or utilization of one or more nutrient components of a fertilizer applied during growth of said plant, wherein said one or more nutrient components is selected from the group consisting of nitrogen, phosphorus, potassium, and iron, and wherein said Methylobacterium isolate is selected from the group consisting of NLS0665 (NRRL B-68194), NLS0754 (NRRL B-68197), NLS0672 (NRRL B-68196), NLS0693 (NRRL B-67926), NLS0729 (NRRL B-68195), NLS0049 (NRRL B-68236), NLS0591 (NRRL B-68215), NLS0439 (NRRL B-68216), NLS1310, NLS1312, NLS0612 (NRRL B-68237), NLS0706 (NRRL B-68238), and NLS0725 (NRRL B-68239).

17. The method of claim 16, wherein the Methylobacterium isoate is selected from the group consisting of NLS0665 (NRRL B-68194), NLS0754 (NRRL B-68197), NLS0672 (NRRL B-68196), NLS0729 (NRRL B-68195), NLS0049 (NRRL B-68236), NLS0591 (NRRL B-68215), and NLS0439 (NRRL B-68216).

18. The method of claim 16 or 17, wherein said fertilizer is applied at reduced rates as compared to standard application rates for said plant.

19. The method of any one of claims 16 to 18, wherein said plant is treated by application with a composition comprising said Methylobacterium and a fertilizer.

20. The method of any one of claims 16 to 19, wherein said plant is an agricultural row crop plant grown in soil.

21. The method of any one of claims 16 to 19, wherein said plant is a leafy green plant.

22. The method of claim 21, wherein said leafy green plant is grown in a hydroponic or aeroponic plant growth system.

23. The method of any one of claims 16 to 19, wherein said plant is a rice plant.

24. An isolated Methylobacterium selected from the group consisting of NLS0665 (NRRL B-68194), NLS0754 (NRRL B-68197), NLS0672 (NRRL B-68196), NLS0729 (NRRL B-68195), NLS0049 (NRRL B-68236), NLS0591 (NRRL B-68215), NLS0439 (NRRL B-68216), NLS1310, NLS1312, NLS0612 (NRRL B-68237), NLS0706 (NRRL B-68238), and NLS0725 (NRRL B-68239).

25. The isolated Methylobacterium of claim 24, selected from the group consisting of NLS0665 (NRRL B-68194), NLS0754 (NRRL B-68197), NLS0672 (NRRL B-68196), NLS0729 (NRRL B-68195), NLS0049 (NRRL B-68236), NLS0591 (NRRL B-68215), and NLS0439 (NRRL B-68216).
